(19) United States
(12) Patent Application Publication
(10) Pub. No.: US 2009/0044345 A1
(43) Pub. Date: Feb. 19, 2009

(54) Use of Metal Complex Compounds as Oxidation Catalysts

(76) Inventors: Gunther Schlingloff, Riehen (CH); Kishor Ramachandra Nivalkar, Mumbai (IN); Torsten Wieprecht, Schopfheim (DE); Marie-Josee Dubs, Wittersdorf (FR); Nicole End, Oberwil (CH)

Correspondence Address: JoAnn Villamizar;Ciba Corporation/Patent Department, 540 White Plains Road, P.O. Box 2005, Tarrytown, NY, 10591, US

(21) Appl. No.: 12/223,429

(22) PCT Filed: Feb. 6, 2006
(86) PCT No.: PCT/EP2006/050701

§ 371 Date: Oct. 9, 2008

(51) Int. Cl. C11D 3/39; D06L 3/00; A01N 59/00; A01P 1/00; C07F 15/02; C07F 15/04; C07F 15/06; C07F 1/08; C07F 7/28; C07D 401/14; C02F 1/68; C02F 103/00

(52) U.S. Cl. 8/111; 510/281; 510/311; 510/220; 424/613; 546/2; 544/319; 546/258; 546/256; 544/364; 210/763

(57) ABSTRACT

The present invention relates to the use, as oxidation catalysts, of metal complex compounds having tetradentate ligands of formula (2) wherein all substitutents have the meanings as defined in Claim 1. The present invention relates also to formulations comprising such metal complex compounds, to novel metal complex compounds and to novel ligands.

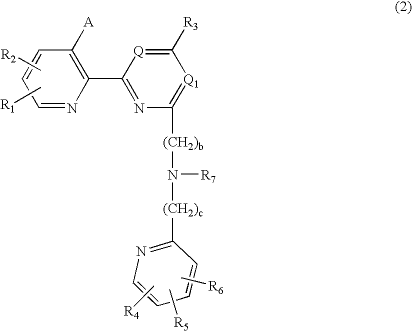

The present invention relates to the use, as oxidation catalysts, of metal complex compounds having tetradentate ligands or mixtures of such ligands. The present invention relates also to formulations comprising such metal complex compounds, to novel metal complex compounds and to novel ligands.

The metal complex compounds are used especially for enhancing the action of peroxides, for example in the treatment of textile material, without at the same time causing any appreciable damage to fibres and dyeings. There is also no appreciable damage to fibres and dyeings if these metal complexes are used in combination with an enzyme or a mixture of enzymes.

The metal complex compounds may also be used as catalysts for oxidation using molecular oxygen and/or air, that is, without peroxide compounds and/or peroxide-forming substances. The bleaching of the fabric can happen during and/or after the treatment of the fibre with the formulation, which comprises the metal complexes.

Peroxide-containing bleaching agents have long been used in washing and cleaning processes. They have an excellent action at a liquor temperature of 90° C. and above, but their performance noticeably decreases with lower temperatures. Various transition metal ions added in the form of suitable salts, and coordination compounds containing such cations are known to activate H₂O₂. In that manner it is possible for the bleaching effect, which is unsatisfactory at lower temperatures, of H₂O₂ or precursors that release H₂O₂ and of other peroxo compounds, to be increased. They are important for practical purposes, in that respect, especially combinations of transition metal ions and ligands of which the peroxide activation is manifested in an increased tendency towards oxidation in relation to substrates and not only in a catalase-like disproportionation. The latter activation, which in the present case tends rather to be undesirable, could even impair the bleaching effects, which are inadequate at low temperatures, of H₂O₂ and its derivatives.

In terms of H₂O₂ activation having effective bleaching action, mononuclear and polynuclear variants of manganese complexes having various ligands, especially 1,4,7-trimethyl-1,4,7-triazacyclononane and optionally oxygen-containing bridging ligands, are currently regarded as being especially effective. Such catalysts are adequately stable under practical conditions and, with Mnⁿ⁺, contain an ecologically acceptable metal cation, but their use is unfortunately associated with considerable damage to dyes and fibres.

The aim of the present invention was accordingly to provide improved metal complex catalysts for oxidation processes that meet the above requirements and, especially, enhance the action of peroxide compounds in the most varied fields of application without causing any appreciable damage.

The invention accordingly relates to the use, as a catalyst for oxidation reactions, of at least one metal complex of formula (1)

[LₙMeₘXₚ]^{z}Y_{q} (1),

wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2)

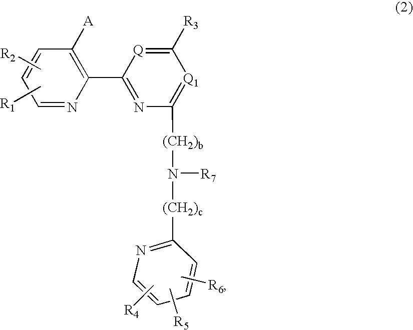

wherein
R₁, R₂, R₃, R₄, R₅, R₆ and R₇ are each independently of the others hydrogen; unsubstituted or substituted C₁-C₁₈alkyl or unsubstituted or substituted aryl; cyano; halogen; nitro; —COOR₉ or —SO₃R₉ wherein
R₉ is in each case hydrogen, a cation or unsubstituted or substituted C₁-C₁₈alkyl or unsubstituted or substituted aryl;
   —SR₁₀, —SO₂R₁₀ or —OR₁₀ wherein
R₁₀ is in each case hydrogen or unsubstituted or substituted C₁-C₁₈alkyl or unsubstituted or substituted aryl;
   —NR₁₁R₁₂; —(C₁-C₆alkylene)-N^{⊕}R₁₁R₁₂; —N^{⊕}R₁₁R₁₂R₁₃; —(C₁-C₆alkylene)-NR₁₁R₁₂R₁₃;
   —N(R₁₀)—(C₁-C₆alkylene)-NR₁₁R₁₂; —N[(C₁-C₆alkylene)-N^{⊕}R₁₁R₁₂]₂;
   —N(R₁₀)—(C₁-C₆alkylene)-N^{⊕}R₁₁R₁₂R₁₃; —N[(C₁-C₆alkylene)-N^{⊕}R₁₁R₁₂R₁₃]₂; —N(R₁₀)—N—R₁₁R₁₂ or
   —N(R₁₀)—N^{⊕}R₁₁R₁₂R₁₃, wherein
R₁₀ is as defined above and
R₁₁, R₁₂ and R₁₃ are each independently of the other(s) hydrogen or unsubstituted or substituted C₁-C₁₈alkyl or unsubstituted or substituted aryl, or
R₁₁ and R₁₂, together with the nitrogen atom linking them, form an unsubstituted or substituted 5-, 6- or 7-membered ring which may contain further hetero atoms,
   Q is N or CR₈, wherein R₈ has the meanings as defined for R₁-R₇ or
R₈ forms together with A a

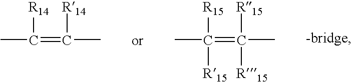

wherein R₁₄, R′₁₄, R₁₅, R′₁₅, R″₁₅ and R′″₁₅ independently from each other are H. C₁-C₄-alkyl or C₁-C₄-alkoxy,
   Q₁ is N or CR′₈, wherein R′₈ has the meanings as defined for R₁-R₇,
   A has one of the meanings as defined for R₁-R₇, or
A forms together with R₈ a

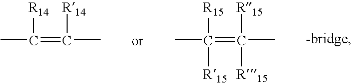

wherein R₁₄, R′₁₄, R₁₅, R′₁₅, R″₁₅ and R′″₁₅ have the same meanings as defined above b and c are each independently from each other 1, 2 or 3.

Suitable substituents for the alkyl groups, aryl groups, alkylene groups or 5-, 6- or 7-membered rings are especially C₁-C₄alkyl; C₁-C₄alkoxy; hydroxy; sulfo; sulfato; halogen; cyano; nitro; carboxy; amino; N-mono- or N,N-di-C₁-C₄alkylamino unsubstituted or substituted by hydroxy in the alkyl moiety; N-phenylamino; N-naphthylamino; phenyl; phenoxy or naphthyloxy.

The C₁-C₁₈alkyl radicals mentioned for the compounds of formula (2) are, for example, straight-chain or branched alkyl radicals, such as methyl, ethyl, n-propyl, isopropyl, n-butyl, sec-butyl, isobutyl, tert-butyl or straight-chain or branched pentyl, hexyl, heptyl or octyl. Preference is given to C₁-C₁₂alkyl radicals, especially C₁-C₈alkyl radicals and preferably C₁-C₄alkyl radicals. The mentioned alkyl radicals may be unsubstituted or substituted e.g. by hydroxy, C₁-C₄alkoxy, sulfo or by sulfato, especially by hydroxy. The corresponding unsubstituted alkyl radicals are preferred. Very special preference is given to methyl and ethyl, especially methyl.

Examples of aryl radicals that come into consideration for the compounds of formula (2) are phenyl or naphthyl each unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, wherein the amino groups may be quaternised, phenyl, phenoxy or by naphthyloxy. Preferred substituents are C₁-C₄alkyl, C₁-C₄alkoxy, phenyl and hydroxy. Special preference is given to the corresponding phenyl radicals.

The C₁-C₆alkylene groups mentioned for the compounds of formula (2) are, for example, straight-chain or branched alkylene radicals, such as methylene, ethylene, n-propylene or n-butylene. C₁-C₄alkylene groups are preferred. The alkylene radicals mentioned may be unsubstituted or substituted, for example by hydroxy or C₁-C₄alkoxy.

In the compounds of formulae (1) and (2), halogen is preferably chlorine, bromine or fluorine, with special preference being given to chlorine.

Examples of cations that come into consideration for compounds of formulae (1) and (2) include alkali metal cations, such as lithium, potassium and especially sodium, alkaline earth metal cations, such as magnesium and calcium, and ammonium cations. The alkali metal cations, especially sodium, are preferred.

Suitable metal ions for Me for the compounds of formula (1) are, for example, manganese in oxidation states II-V, titanium in oxidation states III and IV, iron in oxidation states I to IV, cobalt in oxidation states I to III, nickel in oxidation states I to III and copper in oxidation states I to III, with special preference being given to manganese, especially manganese in oxidation states II to IV, preferably in oxidation state II. Also of interest are titanium IV, iron II-IV, cobalt II-III, nickel II-III and copper II-III, especially iron II-IV.

For the radical X for the compounds of formula (1) there come into consideration, for example, CH₃CN; H₂O; F⁻; Cl⁻; Br⁻; HOO⁻; O₂²⁻; O²⁻; R₁₆COO⁻; R₁₆O⁻; LMeO⁻ and LMeOO⁻, wherein R₁₆ is hydrogen, —SO₃C₁-C₄alkyl or unsubstituted or substituted C₁-C₁₈alkyl or unsubstituted or substituted aryl, and C₁-C₁₈alkyl, aryl, L and Me have the definitions and preferred meanings given hereinabove and hereinbelow. Especially preferably, R₁₆ is hydrogen; C₁-C₄alkyl; sulfophenyl or phenyl, especially hydrogen.

As counter-ion Y for the compounds of formula (1) there come into consideration, for example, R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻ and I⁻, wherein R₁₇ is hydrogen or unsubstituted or substituted C₁-C₁₈alkyl or unsubstituted or substituted aryl. R₁₇ as C₁-C₁₈alkyl or aryl has the definitions and preferred meanings given hereinabove and hereinbelow. Especially preferably, R₁₇ is hydrogen; C₁-C₄alkyl; phenyl or sulfophenyl, especially hydrogen or 4-sulfophenyl. The charge of the counter-ion Y is accordingly preferably 1- or 2-, especially 1-.

Y can also be a customary organic counter-ion, for example citrate, oxalate or tartrate. For the compounds of formula (1), n is preferably an integer having a value of from 1 to 4, preferably 1 or 2 and especially 1.

For the compounds of formula (1), m is preferably an integer having a value of 1 or 2, especially 1.

For the compounds of formula (1), p is preferably an integer having a value of from 0 to 4, especially 2.

For the compounds of formula (1), z is preferably an integer having a value of from 8− to 8+, especially from 4− to 4+ and especially preferably from 0 to 4+. z is more especially the number 0.

For the compounds of formula (1), q is preferably an integer from 0 to 8, especially from 0 to 4, and is especially preferably the number 0.

R₉ in compounds of formula (2) is preferably hydrogen, a cation, C₁-C₁₂alkyl, unsubstituted phenyl or phenyl substituted as indicated above. Especially preferably, R₉ is hydrogen, an alkali metal cation, alkaline earth metal cation or ammonium cation, C₁-C₄alkyl or phenyl, especially hydrogen or an alkali metal cation, alkaline earth metal cation or ammonium cation.

R₁₀ in compounds of formula (2) is preferably hydrogen, C₁-C₁₂alkyl, unsubstituted phenyl or phenyl substituted as indicated above. Especially preferably, R₁₀ is hydrogen, C₁-C₄alkyl or phenyl, more especially hydrogen or C₁-C₄alkyl, preferably hydrogen. Examples of the radical of formula —OR₁₀ that may be mentioned are hydroxy and C₁-C₄alkoxy, such as methoxy and especially ethoxy.

When R₁₁ and R₁₂ in compounds of formula (2), together with the nitrogen atom linking them, form a 5-, 6- or 7-membered ring, that ring is preferably an unsubstituted or C₁-C₄alkyl-substituted imidazole, pyrazole, pyrrolidine, piperidine, piperazine, morpholine or azepane ring, wherein the amino groups may be quaternised, in which case preferably the nitrogen atoms, that are not bonded directly to the pyridine or pyrimidine rings, are quaternised. The piperazine ring may, for example, be substituted by one or two unsubstituted C₁-C₄alkyl and/or substituted C₁-C₄alkyl at the nitrogen atom not bonded to the pyridine ring. In addition, R₁₁, R₁₂ and R₁₃ are preferably hydrogen, unsubstituted or hydroxy-substituted C₁-C₁₂alkyl, unsubstituted phenyl or phenyl substituted as indicated above. Special preference is given to hydrogen, unsubstituted or hydroxy-substituted C₁-C₄alkyl or unsubstituted or hydroxy-substituted phenyl, especially hydrogen or unsubstituted or hydroxy-substituted C₁-C₄alkyl, preferably hydrogen.

R₃ in L of formula (2) is preferably C₁-C₁₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenyl-amino, N-naphthylamino, phenyl, phenoxy or by naphthyloxy; cyano; halogen; nitro; —COOR₉ or —SO₃R₉ wherein R₉ is in each case hydrogen, a cation, C₁-C₁₂alkyl, unsubstituted phenyl or phenyl substituted as indicated above; —SR₁₀, —SO₂R₁₀ or —OR₁₀ wherein R₁₀ is in each case hydrogen, C₁-C₁₂alkyl, unsubstituted phenyl or phenyl substituted as indicated above; —NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃; —N(R₁₀)—(C₁-C₆alkylene)-NR₁₁R₁₂; —N(R₁₀)—(C₁-C₆alkylene)-N^{⊕}R₁₁R₁₂R₁₃, —N(R₁₀)—N^{⊕}R₁₁R₁₂ or —N(R₁₀)—N^{⊕}R₁₁R₁₂R₁₃, wherein R₁₀ may have one of the meanings given above and R₁₁, R₁₂ and R₁₃ are each independently of the other(s) hydrogen, unsubstituted or hydroxy-substituted C₁-C₁₂alkyl, unsubstituted phenyl or phenyl substituted as indicated above, or R₁₁ and R₁₂, together with the nitrogen atom linking them, form a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised.

R₃ in L of formula (2) is especially preferably phenyl unsubstituted or substituted by C₁-C₄alkyl, C₁-C₄alkoxy, halogen, phenyl or by hydroxy; cyano; nitro; —COOR₉ or —SO₃R₉ wherein R₉ is in each case hydrogen, a cation, C₁-C₄alkyl or phenyl; —SR₁₀, —SO₂R₁₀ or —OR₁₀ wherein R₁₀ is in each case hydrogen, C₁-C₄alkyl or phenyl; —N(R₁₀)—CH₂CH₂—R_{α}, wherein R₁₀ has the meaning as defined above and R_{α} is a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised; —N(CH₃)—NH₂ or —NH—NH₂; amino; N-mono- or N,N-di-C₁-C₄alkylamino unsubstituted or substituted by hydroxy in the alkyl moiety; or an unsubstituted or C₁-C₄alkyl-substituted pyrrolidine, piperidine, piperazine, morpholine or azepane ring.

R₃ in L of formula (2) is very especially preferably C₁-C₄alkoxy; hydroxy; phenyl unsubstituted or substituted by C₁-C₄alkyl, C₁-C₄alkoxy, phenyl or by hydroxy; —N(R₁₀)—CH₂CH₂—R_{α}, wherein R₁₀ is H or C₁-C₂alkyl and R_{α} is a imidazole or pyrazole ring unsubstituted or substituted by at least one unsubstituted C₁-C₂alkyl, wherein the nitrogen atom may be quaternised; hydrazine; amino; N-mono- or N,N-di-C₁-C₄alkylamino unsubstituted or substituted by hydroxy in the alkyl moiety; or an unsubstituted or C₁-C₄alkyl-substituted pyrrolidine, piperidine, piperazine, morpholine or azepane ring.

As radicals R₃ in L of formula (2) there are especially important C₁-C₄alkoxy; hydroxy; —N(R₁₀)—CH₂CH₂—R_{α}, wherein R₁₀ is H or C₁-C₂alkyl and R_{α} is a imidazole or pyrazole ring unsubstituted or substituted by at least one unsubstituted C₁-C₂alkyl, wherein the nitrogen atom may be quaternised; hydrazine; amino; N-mono- or N,N-di-C₁-C₄alkylamino unsubstituted or substituted by hydroxy in the alkyl moiety; and an unsubstituted or C₁-C₄alkyl-substituted pyrrolidine, piperidine, piperazine, morpholine or azepane ring.

As radicals R₃ in L of formula (2) there are very especially important C₁-C₄alkoxy; hydroxy; —N(R₁₀)—CH₂CH₂—R_{α}, wherein R₁₀ is H or C₁-C₂alkyl and R_{α} is a imidazole or pyrazole ring unsubstituted or substituted by at least one unsubstituted C₁-C₂alkyl, wherein the nitrogen atom may be quaternised; N-mono- or N,N-di-C₁-C₂alkylamino substituted by hydroxy in the alkyl moiety; and an unsubstituted or C₁-C₂alkyl-substituted pyrrolidine, piperidine, piperazine, morpholine or azepane ring. Of those, hydroxy is of special interest.

The preferred meanings given above for R₃ apply also to R₁, R₂, R₄, R₅, R₆ and R₇ in L of formula (2), but those radicals may additionally be hydrogen.

Q₁ is preferably N; CH; or CR′₈, wherein R′₈ is preferably C₁-C₁₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, phenyl, phenoxy or by naphthyloxy; cyano; halogen; nitro; —COOR₉ or —SO₃R₉ wherein R₉ is in each case hydrogen, a cation, C₁-C₁₂alkyl, unsubstituted phenyl or phenyl substituted as indicated above; —SR₁₀, —SO₂R₁₀ or —OR₁₀ wherein R₁₀ is in each case hydrogen, C₁-C₁₂alkyl, unsubstituted phenyl or phenyl substituted as indicated above; —NR₁₁R₁₂; —(C₁-C₆alkylene)-NR₁₁R₁₂; —N^{⊕}R₁₁R₁₂R₁₃; —(C₁-C₆ alkylene)-N^{⊕}R₁₁R₁₂R₁₃; —N(R₁₀)—(C₁-C₆alkylene)-NR₁₁R₁₂; —N(R₁₀)—(C₁-C₆alkylene)-N^{⊕}R₁₁R₁₂R₁₃, —N(R₁₀)—N—R₁₁R₁₂ or —N(R₁₀)—N^{⊕}R₁₁R₁₂R₁₃, wherein R₁₀ may have one of the meanings given above and R₁₁, R₁₂ and R₁₃ are each independently of the other(s) hydrogen, unsubstituted or hydroxy-substituted C₁-C₁₂alkyl, unsubstituted phenyl or phenyl substituted as indicated above, or R₁₁ and R₁₂, together with the nitrogen atom linking them, form a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised.

Q is preferably N; CH; or CR₈, wherein R₈ is preferably C₁-C₁₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, phenyl, phenoxy or by naphthyloxy; cyano; halogen; nitro; —COOR₉ or —SO₃R₉ wherein R₉ is in each case hydrogen, a cation, C₁-C₁₂alkyl, unsubstituted phenyl or phenyl substituted as indicated above; —SR₁₀, —SO₂R₁₀ or —OR₁₀ wherein R₁₀ is in each case hydrogen, C₁-C₁₂alkyl, unsubstituted phenyl or phenyl substituted as indicated above; —NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃; —N(R₁₀)—(C₁-C₆alkylene)-NR₁₁R₁₂; —N(R₁₀)—(C₁-C₆alkylene)-N^{⊕}R₁₁R₁₂R₁₃, —N(R₁₀)—N—R₁₁R₁₂ or —N(R₁₀)—N^{⊕}R₁₁R₁₂R₁₃, wherein R₁₀ may have one of the meanings given above and R₁₁, R₁₂ and R₁₃ are each independently of the other(s) hydrogen, unsubstituted or hydroxy-substituted C₁-C₁₂alkyl, unsubstituted phenyl or phenyl substituted as indicated above, or R₁₁ and R₁₂, together with the nitrogen atom linking them, form a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised or Q forms together with A a —CH₂—CH₂—, —CH₂—CHR″₁₅—, —CH₂—CR″₁₅R′″₁₅—, —CHR″₁₅—CH₂—, —CHR₁₅—CHR″₁₅—, —CHR₁₅—CR″₁₅R′″₁₅—, —CR₁₅R′₁₅—CH₂—, —CR₁₅R′₁₅—CHR″₁₅—, —CR₁₅R′₁₅—CR″₁₅R′″₁₅—, —CH═CH—, —CR′₁₄═CR′₁₄—, —CH═CR′₁₄— or a —CR₁₄═CH— bridge, wherein R₁₄, R′₁₄, R₁₅, R′₁₅, R″₁₅ and R′″₁₅ independently from each other are C₁-C₂-alkyl.

A is preferably C₁-C₁₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, phenyl, phenoxy or by naphthyloxy; cyano; halogen; nitro; —COOR₉ or —SO₃R₉ wherein R₉ is in each case hydrogen, a cation, C₁-C₁₂alkyl, unsubstituted phenyl or phenyl substituted as indicated above; —SR₁₀, —SO₂R₁₀ or —OR₁₀ wherein R₁₀ is in each case hydrogen, C₁-C₁₂alkyl, unsubstituted phenyl or phenyl substituted as indicated above; —NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃; —N(R₁₀)—(C₁-C₆alkylene)-NR₁₁R₁₂; —N(R₁₀)—(C₁-C₆alkylene)-N^{⊕}R₁₁R₁₂R₁₃, —N(R₁₀)—N—R₁₁R₁₂ or —N(R₁₀)—N^{⊕}R₁₁R₁₂R₁₃, wherein R₁₀ may have one of the meanings given above and R₁₁, R₁₂ and R₁₃ are each independently of the other(s) hydrogen, unsubstituted or hydroxy-substituted C₁-C₁₂alkyl, unsubstituted phenyl or phenyl substituted as indicated above, or R₁₁ and R₁₂, together with the nitrogen atom linking them, form a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised or

A forms together with Q a —CH₂—CH₂—, —CH₂—CHR″₁₅—, —CH₂—CR″₁₅R′″₁₅—, —CHR₁₅—CH₂—, —CHR₁₅—CHR″₁₅—, —CHR₁₅—CR″₁₅R′″₁₅—, —CR₁₅R′₁₅—CH₂—, —CR₁₅R′₁₅—CHR″₁₅—, —CR₁₅R′₁₅—CR″₁₅R′″₁₅—, —CH═CH—, —CR₁₄═CR′₁₄—, —CH═CR′₁₄- or a —CR₁₄═CH— bridge, wherein R₁₄, R′₁₄, R₁₅, R′₁₅, R″₁₅ and R′″₁₅ independently from each other are C₁-C₂-alkyl.

A preferred embodiment of the present invention related to the use, as a catalyst for oxidation reactions, of at least one metal complex of formula (1′),

[L′ₙMe′_{m′}X′_{p′}]^{z}Y′_{q} (1′)

wherein
Me′ is manganese, titanium, iron, cobalt, nickel or copper,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1 or 2, preferably 1,
p′ is an integer having a value of from 0 to 4, especially 2,
z′ is an integer having a value of from 8− to 8+, preferably from 4− to 4+, preferably from 0 to 4+, especially preferably the number 0,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer from 0 to 8, preferably from 0 to 4, more preferably the number 0,
L′ is a ligand of formula (2a), (2b) or (2c)

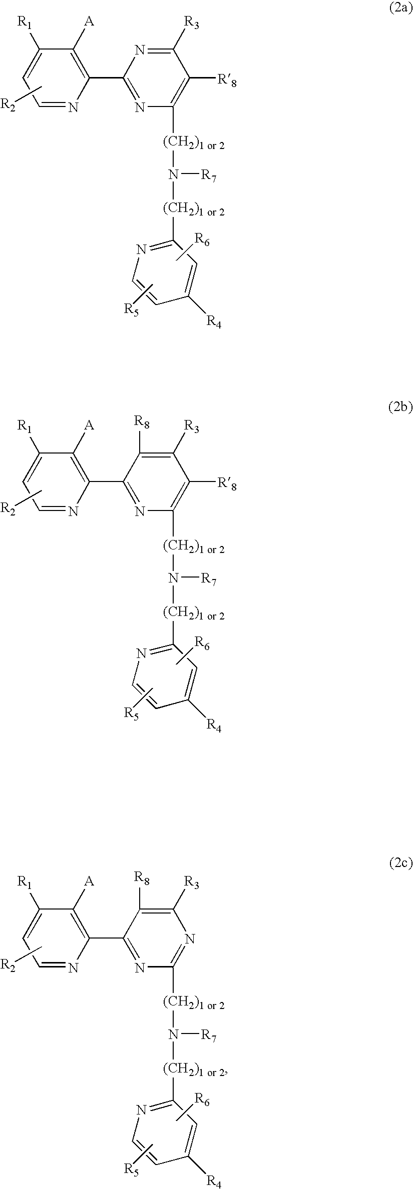

wherein all substituents have the same meanings as defined for formula (2).

A more preferred embodiment of the present invention relates to the use, as a catalyst for oxidation reactions, of at least one metal complex of formula (1′),

[L′ₙMe′_{m′}X′_{p′}]^{z}Y′_{q} (1′)

wherein
Me′ is manganese, titanium, iron, cobalt, nickel or copper,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of from 4− to 4+, preferably from 0 to 4+, especially preferably the number 0,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer from 0 to 4, preferably the number 0,
L′ is a ligand of formula (2a), (2b) or (2d)

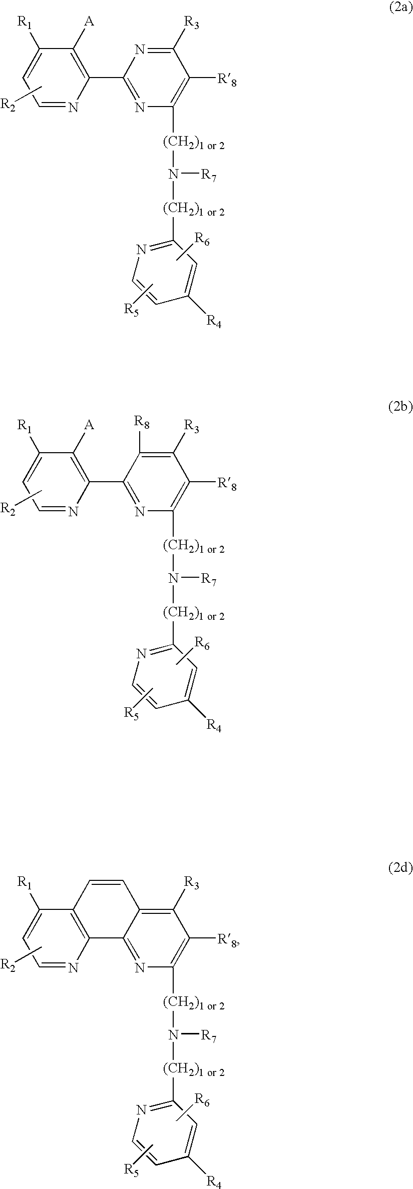

wherein
R₁, R₂, R₄, R₄, R₅, R₆, R₇, R₈, R′₈ and A are independently from each other hydrogen; unsubstituted C₁-C₁₂alkyl; C₁-C₁₂alkyl, which is substituted by at least one substituent chosen from the group consisting of —OH, —CN, —NH₂, COOH and COOC₁-C₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, phenyl, phenoxy or by naphthyloxy; cyano; halogen; nitro; —COOR₉ or —SO₃R₉ wherein R₉ is in each case hydrogen, a cation, C₁-C₁₂alkyl, unsubstituted phenyl or phenyl substituted as indicated above; —SR₁₀, —SO₂R₁₀ or —OR₁₀ wherein R₁₀ is in each case hydrogen, C₁-C₁₂alkyl, unsubstituted phenyl or phenyl substituted as indicated above;
—NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃; —N(R₁₀)—(C₁-C₆alkylene)-NR₁₁R₁₂; —N(R₁₀)—(C₁-C₆alkylene)-N^{⊕}R₁₁R₁₂R₁₃, —N(R₁₀)—N—R₁₁R₁₂ or —N(R₁₀)—N^{⊕}R₁₁R₁₂R₁₃, wherein R₁₀ is hydrogen, C₁-C₄alkyl or phenyl, and R₁₁, R₁₂ and R₁₃ are each independently of the other(s) hydrogen, unsubstituted or hydroxy-substituted C₁-C₁₂alkyl, unsubstituted phenyl or phenyl substituted as indicated above, or R₁₁ and R₁₂, together with the nitrogen atom linking them, form a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised.

As examples of the radical R₃ in L′ of formula (2a), (2b), (2c) and (2d) mention may be made especially of —CH₃; —Cl; —OH; —OCH₃; —CH₂CN; —CH₂CH₂CN; —CH₂COOH; —CH₂CH₂COOH;

### —NH₂; —N(CH₃)₂; —N(CH₂CH₃)₂; —N(CH₃)(CH₂CH₃);

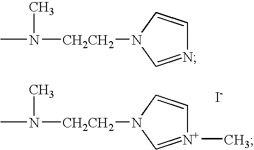

### —N(CH₂CH₂)₂; —N(CH₂CH₃)(CH₂CH₂OH); —N(CH₃)CH₂CH₂OH;

### —N(CH₃)CH₂CH₂NH₂;

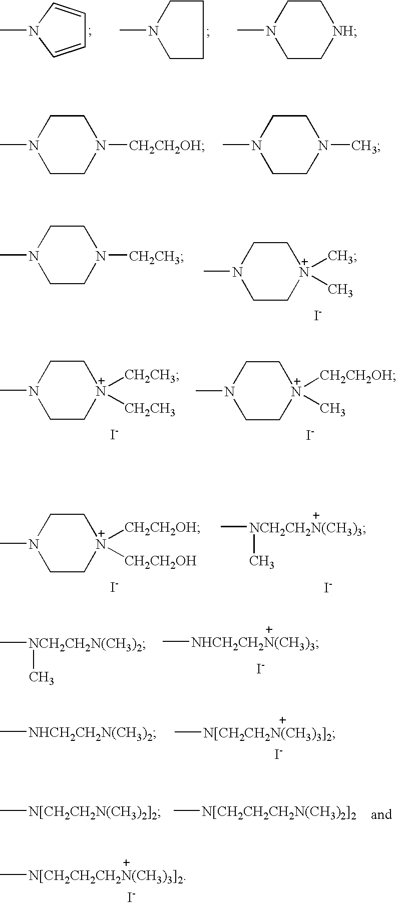

Of those, hydroxy is of special interest.

The preferred meanings given above for R₃ in L′ of formula (2a) and (2b) apply also to R₁, R₂, R₄, R₅, R₆, R₇, R₈, R′₈ and A but those radicals may additionally be hydrogen.

An especially preferred embodiment of the present invention relates to the use, as a catalyst for oxidation reactions, of at least one metal complex of formula (1′),

[L′ₙMe′_{m′}X′_{p′]}^{z}Y′_{q} (1′)

wherein
Me′ is manganese or iron,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of from 0 to 4+, preferably the number 0,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer from 0 to 4, preferably the number 0,
L′ is a ligand of formula (2′a), (2′b) or (2′d)

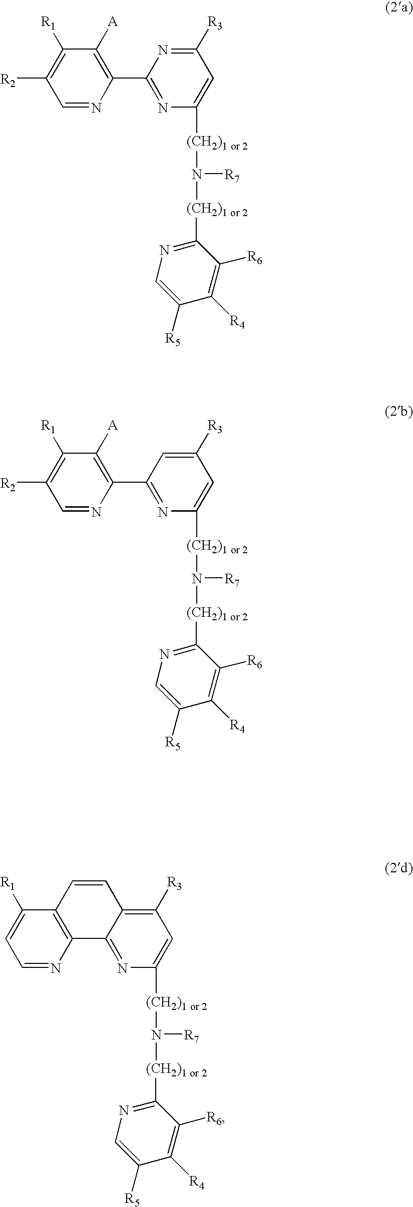

wherein
R₁ and R₄, are independently from each other H; —CH₃; —Cl; —OH; —OCH₃; —NH₂; —N(CH₃)₂;

### —N(CH₂CH₃)₂; —N(CH₃)(CH₂CH₃);

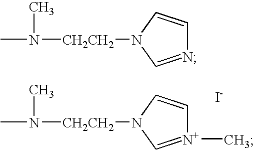

—N(CH₂CH₂OH)₂; —N(CH₂CH₃)(CH₂CH₂OH); —N(CH₃)CH₂CH₂OH; —N(CH₃)CH₂CH₂NH₂;

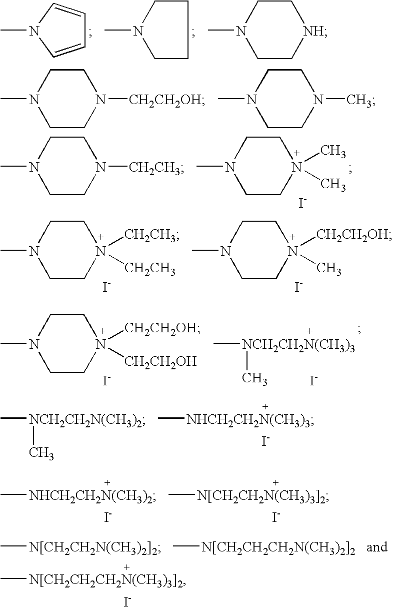

A and R₂, are independently from each other H or —CH₃,

### R₃ is —OH; —OCH₃; —NH₂; —N(CH₃)₂; —N(CH₂CH₃)₂; —N(CH₃)(CH₂CH₃);

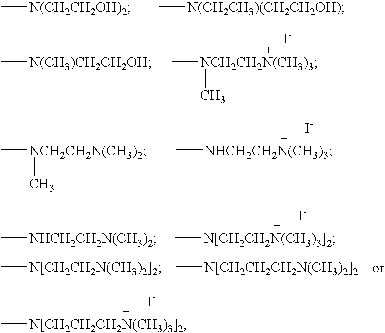

R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and

### R₇ is H; —CH₃; —CH₂COOH; —CH₂CH₂COOH; —CH₂CN or —CH₂CH₂CN.

The metal complex compounds of formula (1) are used together as catalysts with peroxide or a peroxide-forming substance, O₂ and/or air. Examples that may be mentioned in that regard include the following uses:
a) the bleaching of stains or of soiling on textile material in the context of a washing process or by the direct application of a stain remover;
b) the cleaning of hard surfaces, especially kitchen surfaces, wall tiles or floor tiles, for example to remove stains that have formed as a result of the action of moulds (“mould stains”); the use in automatic dishwashing compositions is also a preferred use;
c) the bleaching of stains or of soiling on textile material by atmospheric oxygen, whereby the bleaching is catalysed during and/or after the treatment of the textile in the washing liquor;
d) the prevention of redeposition of migrating dyes during the washing of textile material;
e) use in washing and cleaning solutions having an antibacterial action;
f) as pretreatment agents for bleaching textiles;
g) as catalysts in selective oxidation reactions in the context of organic synthesis;
h) waste water treatment;
i) use as a catalyst for reactions with peroxy compounds for bleaching in the context of paper-making. This relates especially to the delignification of cellulose and bleaching of the pulp, which can be carried out in accordance with customary procedures. Also of interest is the use as a catalyst for reactions with peroxy compounds for the bleaching of waste printed paper;
j) sterilisation and
k) contact lens disinfection.

Preference is given to the bleaching of stains or soiling on textile material; to the cleaning of hard surfaces, especially kitchen surfaces, wall tiles, floor tiles as well as the use in automatic dishwasher formulations; to the bleaching of stains or of soiling on textile material by atmospheric oxygen, whereby the bleaching is catalysed during and/or after the treatment of the textile in the washing liquor; or to the prevention of redeposition of migrating dyes in the context of a washing process

The preferred metals are for these use are manganese and/or iron.

It should be emphasised that the use of metal complex compounds, for example, in the bleaching of textile or hard surface material, does not cause any appreciable damage to fibres and dyeings well as to the hard surface materials, such as table- and kitchen-ware, as well as tiles.

Processes for bleaching stains in any washing liquor are usually carried out by adding to the washing liquor (with H₂O₂ or a precursor of H₂O₂) one or more metal complex compounds of formula (1) or (1′). Alternatively, it is possible to add a detergent that already comprises one or two metal complex compounds. It will be understood that in such an application, as well as in the other applications, the metal complex compounds of formula (1) or (1′) can alternatively be formed in situ, the metal salt (e.g. manganese(II) salt, such as manganese(II) chloride, and/or iron(II) salt, such as iron(II) chloride) and the ligand being added in the desired molar ratios.

The present invention relates also to a detergent, cleaning, disinfecting or bleaching composition comprising
I) from 0-50% by weight (wt-%), preferably from 0-30 wt-%, A) of at least one anionic surfactant and/or B) of a non-ionic surfactant,
II) from 0-70 wt-%, preferably from 0-50 wt-%, C) of at least one builder substance,
III) from 1-99 wt-%, preferably 1-50 wt-%, D) of at least of a peroxide or a peroxide-forming substance, O₂ and/or air, IV) E) at least one metal complex compound of formula (1) or (1′) in an amount that, in the liquor, gives a concentration of from 0.5-100 mg/litre of liquor, preferably from 1-50 mg/litre of liquor, when from 0.5-50 g/litre of the detergent, cleaning, disinfecting or bleaching agent are added to the liquor,
V) from 0-20 wt-% of at least one further additive, and
VI) water ad 100 wt-%.

All wt-% are based on the total weight of the detergent, cleaning, disinfecting or bleaching composition.

The detergent, cleaning, disinfecting or bleaching composition can be any kind of industrial or domestic cleaning, disinfecting or bleaching formulation.

It can be used for example in compositions used for textile material as well as in composition used for hardsurfaces, such as hard surface materials, such as table- and kitchen-ware, as well as tiles.

Preferred hard surface cleaning compositions are dishwashing detergent formulations, more preferably automatic dishwashing detergent formulations.

The above percentages are in each case percentages by weight, based on the total weight of the composition. The compositions preferably contain from 0.005 to 2 wt-% of at least one metal complex compound of formula (1) or (1′), more preferably from 0.01 to 1 wt-% and most preferably from 0.05 to 1 wt-%.

Therefore a further embodiment of the present invention relates to a detergent, cleaning, disinfecting or bleaching composition comprising
I) from 0-50 wt-%, preferably from 0-30 wt-% by, A) of at least one anionic surfactant and/or B) of a non-ionic surfactant,
II) from 0-70 wt-%, preferably from 0-50 wt-%, C) of at least one builder substance,
III) from 1-99 wt-%, preferably 1-50 wt-%, D) of at least one peroxide and/or at least one peroxide-forming substance, O₂ and/or air,
IV) from 0.005-2 wt-%, more preferably from 0.01-1 wt-% and most preferably from 0.05-1 wt-% E) of at least one metal complex compound of formula (1) or (1′) as defined above,
V) from 0-20 wt-% of at least one further additive, and
VI) water ad 100% by weight.

All wt-% are based on the total weight of the detergent, cleaning, disinfecting or bleaching composition.

When the compositions according to the invention comprise a component A) and/or B), the amount thereof is preferably from 1 to 50 wt-%, especially from 1 to 30 wt-%.

Therefore a further embodiment of the present invention relates to a detergent, cleaning, disinfecting or bleaching composition comprising
I) from 1-50 wt-%, preferably from 1-30 wt-%, A) of at least one anionic surfactant and/or B) of at least one non-ionic surfactant,
II) from 0-70 wt-%, preferably from 0-50 wt-%, C) of at least one builder substance,
III) from 1-99 wt-%, preferably 1-50 wt-%, D) of at least one peroxide and/or of at least one peroxide-forming substance, O₂ and/or air,
IV) from 0.005-2 wt-%, more preferably from 0.01-1 wt-% and most preferably from 0.05-1 wt-% E) of at least one metal complex compound of formula (1) or (1′) as defined above,
V) from 0-20 wt-% of at least one further additive, and
VI) water ad 100% by weight.

All wt-% are based on the total weight of the detergent, cleaning, disinfecting or bleaching composition.

When the compositions according to the invention comprise a component C), the amount thereof is preferably from 1 to 70 wt-%, especially from 1 to 50 wt-%. Special preference is given to an amount of from 5 to 50 wt-% and especially an amount of from 10 to 50 wt-%.

Therefore a further embodiment of the present invention relates to a detergent, cleaning, disinfecting or bleaching composition comprising
I) from 1-50 wt-%, preferably from 1-30 wt-%, A) of at least one anionic surfactant and/or B) of at least one non-ionic surfactant,
II) from 1-70 wt-%, preferably from 1-50 wt-%, C) of at least one builder substance,
III) from 1-99 wt-%, preferably 1-50 wt-%, D) of at least one peroxide and/or one peroxide-forming substance, O₂ and/or air,
IV) from 0.005-2 wt-%, more preferably from 0.01-1 wt-% and most preferably from 0.05-1 wt-% E) of at least one metal complex compound of formula (1) or (1′) as defined above,
V) from 0-20 wt-% of at least one further additive, and
VI) water ad 100% by weight.

All wt-% are based on the total weight of the detergent, cleaning, disinfecting or bleaching composition.

Corresponding washing, cleaning, disinfecting or bleaching processes are usually carried out by using an aqueous liquor containing from 0.1 to 200 mg of one or more compounds of formula (1) per litre of liquor. The liquor preferably contains from 1 to 50 mg of at least one compound of formula (1) per litre of liquor.

The composition according to the invention can be, for example, a peroxide-containing heavy-duty detergent or a separate bleaching additive, or a stain remover that is to be applied directly. A bleaching additive is used for removing coloured stains on textiles in a separate liquor before the clothes are washed with a bleach-free detergent. A bleaching additive can also be used in a liquor together with a bleach-free detergent.

Stain removers can be applied directly to the textile in question and are used especially for pretreatment in the event of heavy local soiling.

The stain remover can be applied in liquid form, by a spraying method or in the form of a solid substance, such as a powder especially as a granule.

Granules can be prepared, for example, by first preparing an initial powder by spray-drying an aqueous suspension comprising all the components listed above except for component E), and then adding the dry component E) and mixing everything together. It is also possible to add component E) to an aqueous suspension containing components A), B), C) and D) and then to carry out spray-drying.

It is also possible to start with an aqueous suspension that contains components A) and C), but none or only some of component B). The suspension is spray-dried, then component E) is mixed with component B) and added, and then component D) is mixed in the dry state. It is also possible to mix all the components together in the dry state.

The anionic surfactant A) can be, for example, a sulfate, sulfonate or carboxylate surfactant or a mixture thereof. Preference is given to alkylbenzenesulfonates, alkyl sulfates, alkyl ether sulfates, olefin sulfonates, fatty acid salts, alkyl and alkenyl ether carboxylates or to an α-sulfonic fatty acid salt or an ester thereof.

Preferred sulfonates are, for example, alkylbenzenesulfonates having from 10 to 20 carbon atoms in the alkyl radical, alkyl sulfates having from 8 to 18 carbon atoms in the alkyl radical, alkyl ether sulfates having from 8 to 18 carbon atoms in the alkyl radical, and fatty acid salts derived from palm oil or tallow and having from 8 to 18 carbon atoms in the alkyl moiety. The average molar number of ethylene oxide units added to the alkyl ether sulfates is from 1 to 20, preferably from 1 to 10. The cation in the anionic surfactants is preferably an alkaline metal cation, especially sodium or potassium, more especially sodium. Preferred carboxylates are alkali metal sarcosinates of formula R₁₉—CON(R₂₀)CH₂COOM₁ wherein R₁₉ is C₉-C₁₇alkyl or C₉-C₁₇alkenyl, R₂₀ is C₁-C₄alkyl and M₁ is an alkali metal, especially sodium.

The non-ionic surfactant B) may be, for example, a primary or secondary alcohol ethoxylate, especially a C₈-C₂₀ aliphatic alcohol ethoxylated with an average of from 1 to 20 mol of ethylene oxide per alcohol group. Preference is given to primary and secondary C₁₀-C₁₅ aliphatic alcohols ethoxylated with an average of from 1 to 10 mol of ethylene oxide per alcohol group. Non-ethoxylated non-ionic surfactants, for example alkylpolyglycosides, glycerol monoethers and polyhydroxyamides (glucamide), may likewise be used.

The total amount of anionic and non-ionic surfactants is preferably from 5 to 50 wt-%, especially from 5 to 40 wt-% and more especially from 5 to 30 wt-%. The lower limit of those surfactants to which even greater preference is given is 10 wt-%.

In addition to anionic and/or non-ionic surfactants the composition may contain cationic surfactants. Possible cationic surfactants include all common cationic surface-active compounds, especially surfactants having a textile softening effect.

Non-limited examples of cationic surfactants are given in the formulas below:

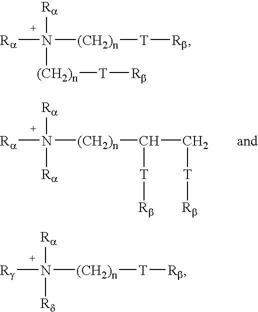

wherein
each radical R_{α} is independent of the others C₁₋₆-alkyl-, -alkenyl- or -hydroxyalkyl;
each radical R_{β} is independent of the others C₈₋₂₈-alkyl- or alkenyl;
R_{γ} is R_{α} or (CH₂)ₙ-T-R_{β};
R_{δ} is R_{α} or R_{β} or (CH₂)ₙ-T-R_{β}; T=—CH₂—, —O—CO— or —CO—O— and
n is between 0 and 5.

Preferred cationic surfactants present in the composition according to the invention include hydroxyalkyl-trialkyl-ammonium-compounds, especially C₁₂₋₁₈-alkyl(hydroxyethyl)dimethylammonium compounds, and especially preferred the corresponding chloride salts. Compositions of the present invention can contain between 0.5 wt-% and 15 wt-% of the cationic surfactant, based on the total weight of the composition.

As builder substance C) there come into consideration, for example, alkali metal phosphates, especially tripolyphosphates, carbonates and hydrogen carbonates, especially their sodium salts, silicates, aluminum silicates, polycarboxylates, polycarboxylic acids, organic phosphonates, aminoalkylenepoly(alkylenephosphonates) and mixtures of such compounds.

Silicates that are especially suitable are sodium salts of crystalline layered silicates of the formula NaHSiₜO₂ₜ₊₁.pH₂O or Na₂SiₜO₂ₜ₊₁.pH₂O wherein t is a number from 1.9 to 4 and p is a number from 0 to 20.

Among the aluminum silicates, preference is given to those commercially available under the names zeolite A, B, X and HS, and also to mixtures comprising two or more of such components. Special preference is given to zeolite A.

Among the polycarboxylates, preference is given to polyhydroxycarboxylates, especially citrates, and acrylates, and also to copolymers thereof with maleic anhydride. Preferred polycarboxylic acids are nitrilotriacetic acid, ethylenediaminetetraacetic acid and ethylene-diamine disuccinate either in racemic form or in the enantiomerically pure (S,S) form.

Phosphonates or aminoalkylenepoly(alkylenephosphonates) that are especially suitable are alkali metal salts of 1-hydroxyethane-1,1-diphosphonic acid, nitrilotris(methylenephosphonic acid), ethylenediaminetetramethylenephosphonic acid and diethylenetriaminepenta-methylenephosphonic acid, and also salts thereof. Also preferred polyphosphonates have the following formula

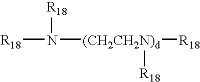

wherein
R₁₈ is CH₂PO₃H₂ or a water soluble salt thereof and
d is an integer of the value 0, 1, 2 or 3.

Especially preferred are the polyphosphonates wherein b is an integer of the value of 1.

The amount of the peroxide or the peroxide-forming substance is preferably 0.5-30 wt-%, more preferably 1-20 wt-% and especially preferably 1-15 wt-%.

As the peroxide component D) there come into consideration every compound which is capable of yielding hydrogen peroxide in aqueous solutions, for example, the organic and inorganic peroxides known in the literature and available commercially that bleach textile materials at conventional washing temperatures, for example at from 10 to 95° C.

Preferably, however, inorganic peroxides are used, for example persulfates, perborates, percarbonates and/or persilicates.

Example of suitable inorganic peroxides are sodium perborate tetrahydrate or sodium perborated monohydrate, sodium percarbonate, inorganic peroxyacid compounds, such as for example potassium monopersulphate (MPS). If organic or inorganic peroxyacids are used as the peroxygen compound, the amount thereof will normally be within the range of about 2-80 wt-%, preferably from 4-30 wt-%.

The organic peroxides are, for example, mono- or poly-peroxides, urea peroxides, a combination of a C₁-C₄alkanol oxidase and C₁-C₄alkanol (Such as methanol oxidase and ethanol as described in WO95/07972), alkylhydroxy peroxides, such as cumene hydroperoxide and t-butyl hydroperoxide.

The peroxides may be in a variety of crystalline forms and have different water contents, and they may also be used together with other inorganic or organic compounds in order to improve their storage stability.

All these peroxy compounds may be utilized alone or in conjunction with a peroxyacid bleach precursor and/or an organic bleach catalyst not containing a transition metal. Generally, the bleaching composition of the invention can be suitably formulated to contain from 2 to 80 wt-%, preferably from 4 to 30 wt-%, of the peroxy bleaching agent.

As oxidants, peroxo acids can also be used. One example are organic mono peracids of formula

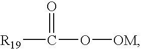

wherein
M signifies hydrogen or a cation,
R₁₉ signifies unsubstituted C₁-C₁₈alkyl; substituted C₁-C₁₈alkyl; unsubstituted aryl; substituted aryl; —(C₁-C₆alkylene)-aryl, wherein the alkylene and/or the alkyl group may be substituted; and phthalimidoC₁-C₈alkylene, wherein the phthalimido and/or the alkylene group may be substituted.

Preferred mono organic peroxy acids and their salts are those of formula

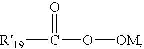

wherein
M signifies hydrogen or an alkali metal, and
R′₁₉ signifies unsubstituted C₁-C₄alkyl; phenyl; —C₁-C₂alkylene-phenyl or phthalimidoC₁-C₈alkylene.

Especially preferred is CH₃COOOH and its alkali salts.

Especially preferred is also ξ-phthalimido peroxy hexanoic acid and its alkali salts.

Also suitable are diperoxyacids, for example, 1,12-diperoxydodecanedioic acid (DPDA), 1,9-diperoxyazelaic acid, diperoxybrassilic acid, diperoxysebasic acid, diperoxyisophthalic acid, 2-decyldiperoxybutane-1,4-diotic acid and 4,4′-sulphonylbisperoxybenzoic acid.

Instead of the peroxy acid it is also possible to use organic peroxy acid precursors and H₂O₂. Such precursors are the corresponding carboxyacid or the corresponding carboxyanhydrid or the corresponding carbonylchlorid, or amides, or esters, which can form the peroxy acids on perhydrolysis. Such reactions are commonly known.

Peroxyacid bleach precursors are known and amply described in literature, such as in the British Patents 836988; 864,798; 907,356; 1,003,310 and 1,519,351; German Patent 3,337,921; EP-A-0185522; EP-A-0174132; EP-A-0120591; and U.S. Pat. Nos. 1,246,339; 3,332,882; 4,128,494; 4,412,934 and 4,675,393.

Peroxy acids precursors are often referred to as bleach activators. Suitable bleach activators include the bleach activators, that carry O- and/or N-acyl groups and/or unsubstituted or substituted benzoyl groups. Preference is given to polyacylated alkylenediamines, especially tetraacetylethylenediamine (TAED); acylated glycolurils, especially tetraacetyl glycol urea (TAGU), N,N-diacetyl-N,N-dimethylurea (DDU); sodium-4-benzoyloxy benzene sulphonate (SBOBS); sodium-1-methyl-2-benzoyloxy benzene-4-sulphonate; sodium-4-methyl-3-benzoloxy benzoate; trimethyl ammonium toluoyloxy-benzene sulphonate; acylated triazine derivatives, especially 1,5-diacetyl-2,4-dioxohexahydro-1,3,5-triazine (DADHT); compounds of formula (6):

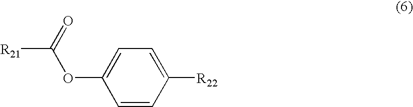

wherein R₂₂ is a sulfonate group, a carboxylic acid group or a carboxylate group, and wherein R₂₁ is linear or branched (C₇-C₁₅)alkyl, especially activators known under the names SNOBS, SLOBS and DOBA; acylated polyhydric alcohols, especially triacetin, ethylene glycol diacetate and 2,5-diacetoxy-2,5-dihydrofuran; and also acetylated sorbitol and mannitol and acylated sugar derivatives, especially pentaacetylglucose (PAG), sucrose polyacetate (SUPA), pentaacetylfructose, tetraacetylxylose and octaacetyllactose as well as acetylated, optionally N-alkylated glucamine and gluconolactone. It is also possible to use the combinations of conventional bleach activators known from German Patent Application DE-A-44 43 177. Nitrile compounds that form perimine acids with peroxides also come into consideration as bleach activators.

Another useful class of peroxyacid bleach precursors is that of the cationic i.e. quaternary ammonium substituted peroxyacid precursors as disclosed in U.S. Pat. Nos. 4,751,015 and 4,397,757, in EP-A0284292 and EP-A-331,229. Examples of peroxyacid bleach precursors of this class are: 2-(N,N,N-trimethyl ammonium) ethyl sodium-4-sulphophenyl carbonate chloride—(SPCC), N-octyl,N,N-dimethyl-N10-carbophenoxy decyl ammonium chloride —(ODC), 3-(N,N,N-trimethyl ammonium) propyl sodium-4-sulphophenyl carboxylate and N,N,N-trimethyl ammonium toluoyloxy benzene sulphonate.

A further special class of bleach precursors is formed by the cationic nitriles as disclosed in EP-A-303,520, WO 96/40661 and in European Patent Specification No.'s 458,396, 790244 and 464,880. These cationic nitriles also known as nitril quats have the formula

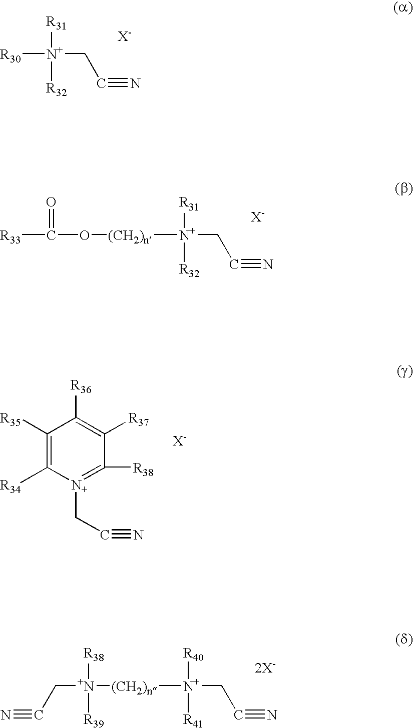

wherein
R₃₀ is a C₁-C₂₄alkyl; a C₁-C₂₄alkenyl; an alkaryl having a C₁-C₂₄alkyl; a substituted C₁-C₂₄alkyl; a substituted C₁-C₂₄alkenyl; a substituted aryl,
R₃₁, and R₃₂ are each independently a C₁-C₃alkyl; hydroxyalkyl having 1 to 3 carbon atoms, —(C₂H₄O)ₙH, n being 1 to 6; —CH₂—CN
R₃₃ is a C₁-C₂₀alkyl; a C₁-C₂₀alkenyl; a substituted C₁-C₂₀alkyl; a substituted C₁-C₂₀alkenyl; an alkaryl having a C₁-C₂₄alkyl and at least one other substituent,
R₃₄, R₃₅, R₃₆, R₃₇ and R₃₈ are each independently hydrogen, a C₁-C₁₀alkyl, a C₁-C₁₀alkenyl, a substituted C₁-C₁₀alkyl, a substituted C₁-C₁₀alkenyl, carboxyl, sulfonyl or cyano
R₃₈, R₃₉, R₄₀ and R₄₁, are each independently a C₁-C₆alkyl,
n′ is an integer from 1 to 3,
n″ is an integer from 1 to 16, and
X is an anion.

Other nitril quats have the following formula

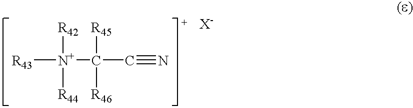

wherein
R₄₂ and R₄₃ form, together with the nitrogen atom to which they are bonded, a ring comprising 4 to 6 carbon atoms, this ring may also be substituted by C₁-C₅-alkyl, C₁-C₅-alkoxy, C₁-C₅-alkanoyl, phenyl, amino, ammonium, cyano, cyanoamino or chloro and 1 or 2 carbon atom(s) of this ring may also be substituted by a nitrogen atom, by a oxygen atom, by a N—R₄₇-group and/or by a R₄₄—N—R₄₇-group, wherein R₄₇ is hydrogen, C₁-C₅-alkyl, C₂-C₅-alkenyl, C₂-C₅-alkinyl, phenyl, C₇-C₉-aralkyl, C₅-C₇-cycloalkyl, C₁-C₅-alkanoyl, cyanomethyl or cyano,
R₄₄ is C₁-C₂₄—, preferably C₁-C₄-alkyl; C₂-C₂₄-alkenyl, preferably C₂-C₄-alkenyl, cyanomethyl or C₁-C₄-alkoxy-C₁-C₄-alkyl,
R₄₅ and R₄₆ are independently from each other hydrogen; C₁-C₄-alkyl; C₁-C₄-alkenyl; C₁-C₄-alkoxy-C₁-C₄-alkyl; phenyl or C₁-C₃-alkylphenyl, preferably hydrogen, methyl or phenyl, whereby preferably the moiety R₄₅ signifies hydrogen, if R₄₆ is not hydrogen, and
X⁻ is an anion.

Suitable examples of nitril quats of formula (ξ) are

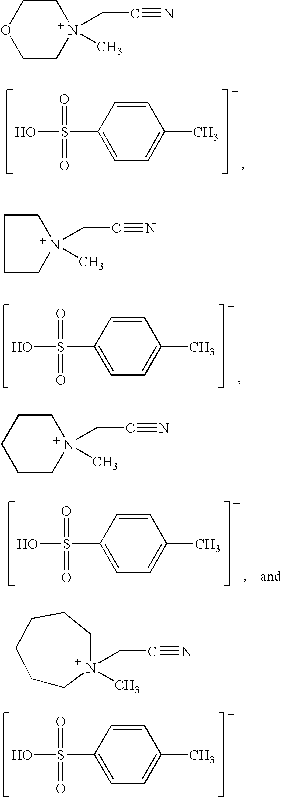

Other nitrile quats have the formula

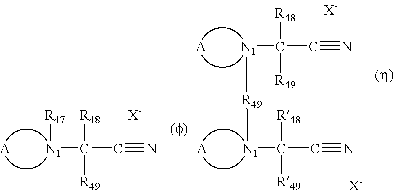

wherein
A is a saturated ring formed by a plurality of atoms in addition to the N₁ atom, the saturated ring atoms to include at least one carbon atom and at least one heteroatom in addition to the N₁ atom, the said one heteroatom selected from the group consisting of O, S and N atoms, the substituent R₄₇ bound to the N₁ atom of the Formula (φ) structure is (a) a C₁-C₈-alkyl or alkoxylated alkyl where the alkoxy is C₂₋₄, (b) a C₄-C₂₄cycloalkyl, (c) a C₇-C₂₄alkaryl, (d) a repeating or nonrepeating alkoxy or alkoxylated alcohol, where the alkoxy unit is C₂₋₄, or (e) —CR₅₀R₅₁—C≡N where R₅₀ and R₅₁ are each H, a C₁-C₂₄alkyl, cycloalkyl, or alkaryl, or a repeating or nonrepeating alkoxyl or alkoxylated alcohol where the alkoxy unit is C₂-C₄, in Formula (φ) at least one of the R₄₈ and R₄₉ substituents is H and the other of R₄₈ and R₄₉ is H, a C₁-C₂₄alkyl, cycloalkyl, or alkaryl, or a repeating or nonrepeating alkoxyl or alkoxylated alcohol where the alkoxy unit is C₂₋₄, and Y is at least one counterion.

The precursors may be used in an amount of up to 12 wt-%, preferably from 2-10 wt-% based on the total weight of the composition.

It is also possible to use further bleach catalysts, which are commonly known, for example transition metal complexes as disclosed in EP 1194514, EP 1383857 or WO04/007657.

It is possible to use H₂O₂, O₂, air, the peroxy-containing compounds, the peroxy-acids as well as their precursors, further bleach catalyst and bleach activists in any combination with the inventive metal complexes.

The compositions may comprise, in addition to the combination according to the invention, one or more optical brighteners, for example from the classes bis-triazinylamino-stilbenedisulfonic acid, bis-triazolyl-stilbenedisulfonic acid, bis-styryl-biphenyl or bis-benzofuranylbiphenyl, α bis-benzoxalyl derivative, bis-benzimidazolyl derivative or coumarin derivative or a pyrazoline derivative.

The compositions may furthermore comprise one or more further additives. Such additives are, for example, dirt-suspending agents, for example sodium carboxymethylcellulose; pH regulators, for example alkali metal or alkaline earth metal silicates; foam regulators, for example soap; salts for adjusting the spray drying and the granulating properties, for example sodium sulfate; perfumes; and also, if appropriate, antistatics and softening agents such as, for example, smectite; bleaching agents; pigments; and/or toning agents. These constituents should especially be stable to any bleaching agent employed.

If the detergent composition is used in an automatic dishwasher it is also common to use silver-corrosion inhibitors.

Such auxiliaries are added in a total amount of from 0.1-20 wt-%, preferably from 0.5-10 wt-%, especially from 0.5-5 wt-%, based on the total weight of the detergent formulation.

Furthermore, the detergent may optionally also comprise enzymes. Enzymes can be added for the purpose of stain removal. The enzymes usually improve the action on stains caused by protein or starch, such as, for example, blood, milk, grass or fruit juices. Preferred enzymes are cellulases and proteases, especially proteases. Cellulases are enzymes that react with cellulose and its derivatives and hydrolyse them to form glucose, cellobiose and cellooligosaccharides. Cellulases remove dirt and, in addition, have the effect of enhancing the soft handle of the fabric.

Examples of customary enzymes include, but are by no means limited to, the following: proteases as described in U.S. Pat. No. 6,242,405, column 14, lines 21 to 32;

lipases as described in U.S. Pat. No. 6,242,405, column 14, lines 33 to 46;

amylases as described in U.S. Pat. No. 6,242,405, column 14, lines 47 to 56; and

cellulases as described in U.S. Pat. No. 6,242,405, column 14, lines 57 to 64.

Commercially available detergent proteases, such as Alcalase®, Esperase®, Everlase®, Savinase®, Kannase® and Durazym®, are sold e.g. by NOVOZYMES A/S.

Commercially available detergent amylases, such as Termamyl®, Duramyl®, Stainzyme®, Natalase®, Ban® and Fungamyl®, are sold e.g. by NOVOZYMES A/S.

Commercially available detergent cellulases, such as Celluzyme®, Carezyme® and Endolase®, are sold e.g. by NOVOZYMES A/S.

Commercially available detergent lipases, such as Lipolase®, Lipolase Ultra® and Lipoprime®, are sold e.g. by NOVOZYMES A/S.

Suitable mannanases, such as Mannanaway®, are sold by NOVOZYMES A/S.

Beside in laundry care products, in a hard surface cleaner, especially in a composition used in automatic dishwashers the following enzymes are also commonly used: proteases, amylases, pullulanases, cutinases and lipases, for example proteases such as BLAP®, Optimase®, Opticlean®, Maxacal®, Maxapem®, Esperase® and/or Savinase®, amylases such as Termamyl®, Amylase-LT®, Maxamyl® and/or Duramyl®, lipases such as Lipolase®, Lipomax®, Lumafast® and/or Lipozym®. The enzymes which may be used can, as described e.g. in International Patent Applications WO 92/11347 and WO 94/23005, be adsorbed on carriers and/or embedded in encapsulating substances in order to safeguard them against premature inactivation. They are present in the cleaning formulations according to the invention preferably in amounts not exceeding 5 wt-%, especially in amounts of from 0.1 wt-% to 1.2 wt-%.

Amylases: The present invention preferably makes use of amylases having improved stability in detergents, especially improved oxidative stability. Such amylases are non-limitingly illustrated by the following: (a) An amylase according to WO 94/02597, Novo Nordisk A/S, published Feb. 3, 1994, as further illustrated by a mutant in which substitution is made, using alanine or threonine (preferably threonine), of the methionine residue located in position 197 of the B. licheniformis alpha-amylase, known as TERMAMYL®, or the homologous position variation of a similar parent amylase, such as B. amyloliquefaciens, B. subtilis, or B. stearothermophilus; (b) Stability-enhanced amylases as described by Genencor International in a paper entitled “Oxidatively Resistant alpha-Amylases” presented at the 207th American Chemical Society National Meeting, Mar. 13-17, 1994, by C. Mitchinson. Therein it was noted that bleaches in automatic dishwashing detergents inactivate alpha-amylases but that improved oxidative stability amylases have been made by Genencor from B. licheniformis NCIB8061. Any other oxidative stability-enhanced amylase can be used. Proteases: Protease enzymes are usually present in preferred embodiments of the invention at levels between 0.001 wt-% and 5 wt-%. The proteolytic enzyme can be of animal, vegetable or microorganism (preferred) origin. More preferred is serine proteolytic enzyme of bacterial origin. Purified or nonpurified forms of enzyme may be used. Proteolytic enzymes produced by chemically or genetically modified mutants are included by definition, as are close structural enzyme variants. Suitable commercial proteolytic enzymes include Alcalase®, Esperase®, Durazyme®, Savinase®, Maxatase®, Maxacal®, and Maxapem® 15 (protein engineered Maxacal). Purafect® and subtilisin BPN and BPN′ are also commercially available.

When present, lipases comprise from about 0.001 wt-% to about 0.01 wt-% of the instant compositions and are optionally combined with from about 1 wt-% to about 5 wt-% of a surfactant having limesoap-dispersing properties, such as an alkyldimethylamine N-oxide or a sulfobetaine. Suitable lipases for use herein include those of bacterial, animal and fungal origin, including those from chemically or genetically modified mutants.

When incorporating lipases into the instant compositions, their stability and effectiveness may in certain instances be enhanced by combining them with small amounts (e.g., less than 0.5 wt-% of the composition) of oily but non-hydrolyzing materials.

The enzymes, when used, may be present in a total amount of from 0.01 to 5 wt-%, especially from 0.05 to 5 wt-% and more especially from 0.1 to 4 wt-%, based on the total weight of the detergent formulation.

If the detergent formulation is a hard surface cleaning composition, preferably a dishwashing detergent formulation, more preferably an automatic dishwashing detergent formulation, then it can optionally also comprises from about 0.001 wt-% to about 10 wt-%, preferably from about 0.005 wt-% to about 8 wt-%, most preferably from about 0.01 wt-% to about 6 wt-% of an enzyme stabilizing system. The enzyme stabilizing system can be any stabilizing system which is compatible with the detersive enzyme. Such a system may be inherently provided by other formulation actives, or be added separately, e.g., by the formulator or by a manufacturer of detergent-ready enzymes. Such stabilizing systems can, for example, comprise calcium ion, boric acid, propylene glycol, short chain carboxylic acids, boronic acids, and mixtures thereof, and are designed to address different stabilization problems depending on the type and physical form of the detergent composition.

In order to enhance the bleaching action, the compositions may, in addition to comprising the catalysts described herein, also comprise photocatalysts the action of which is based on the generation of singlet oxygen.

Further preferred additives to the compositions according to the invention are dye-fixing agents and/or polymers which, during the washing of textiles, prevent staining caused by dyes in the washing liquor that have been released from the textiles under the washing conditions. Such polymers are preferably polyvinylpyrrolidones, polyvinylimidazoles or polyvinylpyridine-N-oxides, which may have been modified by the incorporation of anionic or cationic substituents, especially those having a molecular weight in the range of from 5000 to 60 000, more especially from 10 000 to 50 000. Such polymers are usually used in a total amount of from 0.01 to 5 wt-%, especially from 0.05 to 5 wt-%, more especially from 0.1 to 2 wt-%, based on the total weight of the detergent formulation. Preferred polymers are those mentioned in WO-A-02/02865 (see especially page 1, last paragraph and page 2, first paragraph) and those in WO-A-04/05688.

When the inventive detergent composition is used as hardsurface cleaner, especially when the composition is used in automatic dishwasher formulation then, it has been found out, that it is preferable to avoid the use of simple calcium-precipitating soaps as antifoams in the present compositions as they tend to deposit on the dishware. Indeed, phosphate esters are not entirely free of such problems and the formulator will generally choose to minimize the content of potentially depositing antifoams in the instant compositions.

Other examples for foam suppressors are paraffin, paraffin/alcohol combinations, or bisfatty acid amides.

The hard surface cleaning compositions, preferably dishwashing detergent formulations, more preferably automatic dishwashing detergent formulations herein may also optionally contain one or more heavy metal chelating agents, such as hydroxyethyldiphosphonate (HEDP). More generally, chelating agents suitable for use herein can be selected from the group consisting of amino carboxylates, amino phosphonates, polyfunctionally-substituted aromatic chelating agents and mixtures thereof. Other suitable chelating agents for use herein are the commercial DEQUEST series, and chelants from Nalco, Inc. Aminocarboxylates useful as optional chelating agents include ethylenediaminetetracetates, N-hydroxyethylethylenediaminetriacetates, nitrilotriacetates, ethylenediamine tetraproprionates, triethylenetetraaminehexacetates, diethylenetriamine-pentaacetates, and ethanoldiglycines, alkali metal, ammonium, and substituted ammonium salts thereof and mixtures thereof.

Aminophosphonates are also suitable for use as chelating agents in the compositions of the invention when at least low levels of total phosphorus are permitted in detergent compositions, and include ethylenediaminetetrakis (methylenephosphonates). Preferably, these aminophosphonates do not contain alkyl or alkenyl groups with more than about 6 carbon atoms.

A highly preferred biodegradable chelator for use herein is ethylenediamine disuccinate (“EDDS”).

If utilized, these chelating agents or transition-metal selective sequestrants will generally comprise from about 0.001 wt-% to about 10 wt-%, more preferably from about 0.05 wt-% to about 1 wt-% of the hard surface cleaning compositions, preferably dishwashing detergent formulations, more preferably automatic dishwashing detergent formulations herein.

Preferred hard surface cleaning compositions, preferably dishwashing detergent formulations, more preferably automatic dishwashing detergent formulations herein may additionally contain a dispersant polymer. When present, a dispersant polymer is typically at levels in the range from 0 wt-% to about 25 wt-%, preferably from about 0.5 wt-% to about 20 wt-%, more preferably from about 1 wt-% to about 8 wt-% of the detergent composition. Dispersant polymers are useful for improved filming performance of the present dishwasher detergent compositions, especially in higher pH embodiments, such as those in which wash pH exceeds about 9.5. Particularly preferred are polymers, which inhibit the deposition of calcium carbonate or magnesium silicate on dishware.

Suitable polymers are preferably at least partially neutralized or alkali metal, ammonium or substituted ammonium (e.g., mono-, di- or triethanolammonium) salts of polycarboxylic acids. The alkali metal, especially sodium salts are most preferred. While the molecular weight of the polymer can vary over a wide range, it preferably is from about 1,000 to about 500,000, more preferably is from about 1,000 to about 250,000.

Unsaturated monomeric acids that can be polymerized to form suitable dispersant polymers include acrylic acid, maleic acid (or maleic anhydride), fumaric acid, itaconic acid, aconitic acid, mesaconic acid, citraconic acid and methylenemalonic acid. The presence of monomeric segments containing no carboxylate radicals such as methyl vinyl ether, styrene, ethylene, etc. is suitable provided that such segments do not constitute more than about 50 wt-% of the dispersant polymer.

Copolymers of acrylamide and acrylate having a molecular weight of from about 3,000 to about 100,000, preferably from about 4,000 to about 20,000, and an acrylamide content of less than about 50 wt-%, preferably less than about 20 wt-% of the dispersant polymer can also be used. Most preferably, such dispersant polymer has a molecular weight of from about 4,000 to about 20,000 and an acrylamide content of from about 0 wt-% to about 15 wt-%, based on the total weight of the polymer.

Particularly preferred dispersant polymers are low molecular weight modified polyacrylate copolymers. Such copolymers contain as monomer units: a) from about 90 wt-% to about 10 wt-%, preferably from about 80 wt-% to about 20 wt-% acrylic acid or its salts and b) from about 10 wt-% to about 90 wt-%, preferably from about 20 wt-% to about 80 wt-% of a substituted acrylic monomer or its salt and have the general formula: —[(C(Rₐ)C(R_{b})(C(O)OR_{c})] wherein the apparently unfilled valencies are in fact occupied by hydrogen and at least one of the substituents Rₐ, R_{b}, or R_{c}, preferably Rₐ or R_{b}, is a 1 to 4 carbon alkyl or hydroxyalkyl group; Rₐ or R_{b} can be a hydrogen and R_{c} can be a hydrogen or alkali metal salt. Most preferred is a substituted acrylic monomer wherein Rₐ is methyl, R_{b} is hydrogen, and R_{c} is sodium.

A suitable low molecular weight polyacrylate dispersant polymer preferably has a molecular weight of less than about 15,000, preferably from about 500 to about 10,000, most preferably from about 1,000 to about 5,000. The most preferred polyacrylate copolymer for use herein has a molecular weight of about 3,500 and is the fully neutralized form of the polymer comprising about 70 wt-% acrylic acid and about 30 wt-% methacrylic acid.

Other dispersant polymers useful herein include the polyethylene glycols and polypropylene glycols having a molecular weight of from about 950 to about 30,000.

Yet other dispersant polymers useful herein include the cellulose sulfate esters such as cellulose acetate sulfate, cellulose sulfate, hydroxyethyl cellulose sulfate, methylcellulose sulfate, and hydroxypropylcellulose sulfate. Sodium cellulose sulfate is the most preferred polymer of this group.

Other suitable dispersant polymers are the carboxylated polysaccharides, particularly starches, celluloses and alginates.

Yet another group of acceptable dispersants are the organic dispersant polymers, such as polyaspartate.

Depending on whether a greater or lesser degree of compactness is required, filler materials can also be present in the instant hard surface cleaning compositions, preferably dishwashing detergent formulations, more preferably automatic dishwashing detergent formulations. These include sucrose, sucrose esters, sodium sulfate, potassium sulfate, etc., in amounts up to about 70 wt-%, preferably from 0 wt-% to about 40 wt-% of the hard surface cleaning compositions, preferably dishwashing detergent formulations, more preferably automatic dishwashing detergent formulations. Preferred filler is sodium sulfate, especially in good grades having at most low levels of trace impurities.

Sodium sulfate used herein preferably has a purity sufficient to ensure it is non-reactive with bleach; it may also be treated with low levels of sequestrants, such as phosphonates or EDDS in magnesium-salt form. Note that preferences, in terms of purity sufficient to avoid decomposing bleach, applies also to pH-adjusting component ingredients, specifically including any silicates used herein.

Organic solvents that can be used in the cleaning formulations according to the invention, especially when the latter are in liquid or paste form, include alcohols having from 1 to 4 carbon atoms, especially methanol, ethanol, isopropanol and tert-butanol, diols having from 2 to 4 carbon atoms, especially ethylene glycol and propylene glycol, and mixtures thereof, and the ethers derivable from the mentioned classes of compound. Such water-miscible solvents are present in the cleaning formulations according to the invention preferably in amounts not exceeding 20 wt-%, especially in amounts of from 1 wt-% to 15 wt-%.

Many hard surface cleaning compositions, preferably dishwashing detergent formulations, more preferably automatic dishwashing detergent formulations herein will be buffered, i.e., they are relatively resistant to pH drop in the presence of acidic soils. However, other compositions herein may have exceptionally low buffering capacity, or may be substantially unbuffered. Techniques for controlling or varying pH at recommended usage levels more generally include the use of not only buffers, but also additional alkalis, acids, pH-jump systems, dual compartment containers, etc., and are well known to those skilled in the art. Certain hard surface cleaning compositions, preferably dishwashing detergent formulations, more preferably automatic dishwashing detergent formulations, comprise a pH-adjusting component selected from water-soluble alkaline inorganic salts and water-soluble organic or inorganic builders. The pH-adjusting components are selected so that when the hard surface cleaning composition, preferably dishwashing detergent formulation, more preferably automatic dishwashing detergent formulation is dissolved in water at a concentration of 1,000-5,000 ppm, the pH remains in the range of above about 8, preferably from about 9.5 to about 11. The preferred nonphosphate pH-adjusting component can be selected from the group consisting of:
(i) sodium carbonate or sesquicarbonate;
(ii) sodium silicate, preferably hydrous sodium silicate having SiO₂:Na₂O ratio of from about 1:1 to about 2:1, and mixtures thereof with limited quantities of sodium metasilicate;
(iii) sodium citrate;
(iv) citric acid;
(v) sodium bicarbonate;
(vi) sodium borate, preferably borax;
(vii) sodium hydroxide; and
(viii) mixtures of (i)-(vii).

Preferred embodiments contain low levels of silicate (i.e. from about 3 wt-% to about 10 wt-% SiO₂).

Illustrative of highly preferred pH-adjusting component systems of this specialized type are binary mixtures of granular sodium citrate with anhydrous sodium carbonate, and three-component mixtures of granular sodium citrate trihydrate, citric acid monohydrate and anhydrous sodium carbonate.

The amount of the pH adjusting component in compositions used for automatic dishwashing is preferably from about 1 wt-% to about 50 wt-% of the composition. In a preferred embodiment, the pH-adjusting component is present in the composition in an amount from about 5 wt-% to about 40 wt-%, preferably from about 10 wt-% to about 30 wt-%.

For compositions herein having a pH between about 9.5 and about 11 of the initial wash solution, particularly preferred automatic dishwashing detergent formulations embodiments comprise, by weight of the automatic dishwashing detergent formulations, from about 5 wt-% to about 40 wt-%, preferably from about 10 wt-% to about 30 wt-%, most preferably from about 15 wt-% to about 20 wt-%, of sodium citrate with from about 5 wt-% to about 30 wt-%, preferably from about 7 wt-% to 25 wt-%, most preferably from about 8 wt-% to about 20 wt-% sodium carbonate.

The essential pH-adjusting system can be complemented (i.e. for improved sequestration in hard water) by other optional detergency builder salts selected from nonphosphate detergency builders known in the art, which include the various water-soluble, alkali metal, ammonium or substituted ammonium borates, hydroxysulfonates, polyacetates, and polycarboxylates. Preferred are the alkali metals, especially sodium, salts of such materials. Alternate water-soluble, non-phosphorus organic builders can be used for their sequestering properties. Examples of polyacetate and polycarboxylate builders are the sodium, potassium, lithium, ammonium and substituted ammonium salts of ethylenediamine tetraacetic acid; nitrilotriacetic acid, tartrate monosuccinic acid, tartrate disuccinic acid, oxydisuccinic acid, carboxymethoxysuccinic acid, mellitic acid, and sodium benzene polycarboxylate salts.

The detergent formulations can take a variety of physical forms such as, for example, powder granules, tablets (tabs), gel and liquid. Examples thereof include, inter alia, conventional high-performance detergent powders, supercompact high-performance detergent powders and tabs. One important physical form is the so-called concentrated granular form, which is added to a washing machine.

Also of importance are so-called compact or supercompact detergents. In the field of detergent manufacture, there is a trend towards the production of such detergents that contain an increased amount of active substances. In order to minimize energy consumption during the washing procedure, compact or supercompact detergents need to act effectively at low washing temperatures, for example below 40° C., or even at room temperature (25° C.). Such detergents usually contain only small amounts of fillers or of substances, such as sodium sulfate or sodium chloride, required for detergent manufacture. The total amount of such substances is usually from 0 to 10 wt-%, especially from 0 to 5 wt-%, more especially from 0 to 1 wt-%, based on the total weight of the detergent formulation. Such (super)compact detergents usually have a bulk density of from 650 to 1000 g/l, especially from 700 to 1000 g/l and more especially from 750 to 1000 g/l.

The detergent formulations can also be in the form of tablets (tabs). The advantages of tabs reside in the ease of dispensing and convenience in handling. Tabs are the most compact form of solid detergent formulation and usually have a volumetric density of, for example, from 0.9 to 1.3 kg/litre. To achieve rapid dissolution, such tabs generally contain special dissolution aids:
carbonate/hydrogen carbonate/citric acid as effervescents;
disintegrators, such as cellulose, carboxymethyl cellulose or cross-linked poly(N-vinyl-pyrrolidone);
rapidly dissolving materials, such as sodium (potassium) acetates, or sodium (potassium) citrates;
rapidly dissolving, water-soluble, rigid coating agents, such as dicarboxylic acids.

The tabs may also comprise combinations of such dissolution aids.

The detergent formulation may also be in the form of an aqueous liquid containing from 5 wt-% to 50 wt-%, preferably from 10 wt-% to 35 wt-%, of water or in the form of a non-aqueous liquid containing no more than 5 wt-%, preferably from 0 wt-% to 1 wt-% of water. Non-aqueous liquid detergent formulations may comprise other solvents as carriers. Low molecular weight primary or secondary alcohols, for example methanol, ethanol, propanol and isopropanol, are suitable for that purpose. The solubilising surfactant used is preferably a monohydroxy alcohol but polyols, such as those containing from 2 to 6 carbon atoms and from 2 to 6 hydroxy groups (e.g., 1,3-propanediol, ethylene glycol, glycerol and 1,2-propanediol) can also be used. Such carriers are usually used in a total amount of from 5 wt-% to 90 wt-%, preferably from 10 wt-% to 50 wt-%, based on the total weight of the detergent formulation. The detergent formulations can also used in so-called “unit liquid dose” form.

The invention relates also to granules that comprise the catalysts according to the invention and are suitable for incorporation into a powder-form or granular detergent, cleaning or bleaching composition. Such granules preferably comprise:
a) from 1 wt-% to 99 wt-%, preferably from 1 wt-% to 40 wt-%, especially from 1 wt-% to 30 wt-%, of at least one metal complex compound of formula (1) and of at least one peroxide,
b) from 1 wt-% to 99 wt-%, preferably from 10 wt-% to 99 wt-%, especially from 20 wt-% to 80 wt-%, of at least one binder,
c) from 0 wt-% to 20 wt-%, especially from 1 to 20 wt-%, of at least one encapsulating material,
d) from 0 wt-% to 20 wt-% of at least one further additive and
e) from 0 wt-% to 20 wt-% water.

All wt-% are based on the total weight of the granule.

Or as an alternative the granule can comprise
a) from 1 wt-% to 99 wt-%, preferably from 1 wt-% to 40 wt-%, especially from 1 wt-% to 30 wt-%, of at least one metal complex compound of formula (1) and of at least one peroxide-forming substance,
b) from 1 wt-% to 99 wt-%, preferably from 10 wt-% to 99 wt-%, especially from 20 wt-% to 80 wt-%, of at least one binder,
c) from 0 wt-% to 20 wt-%, especially from 1 to 20 wt-%, of at least one encapsulating material,
d) from 0 wt-% to 20 wt-% of at least one further additive and
e) from 0 wt-% to 20 wt-% water.

All wt-% are based on the total weight of the granule.

For metal complex compound of formula (1) and the peroxide or the peroxide-forming substance as described above [component a)] all preferences as defined above apply for the granule. It also possible to granule the catalyst as such together with suitable granulate material.

As binder (b) there come into consideration water-soluble, dispersible or water-emulsifiable anionic dispersants, non-ionic dispersants, polymers and waxes.

The anionic dispersants used are, for example, commercially available water-soluble anionic dispersants for dyes, pigments etc.

The following products, especially, come into consideration: condensation products of aromatic sulfonic acids and formaldehyde, condensation products of aromatic sulfonic acids with unsubstituted or chlorinated diphenyls or diphenyl oxides and optionally formaldehyde, (mono-/di-)alkylnaphthalenesulfonates, sodium salts of polymerised organic sulfonic acids, sodium salts of polymerised alkylnaphthalenesulfonic acids, sodium salts of polymerised alkylbenzenesulfonic acids, alkylarylsulfonates, sodium salts of alkyl polyglycol ether sulfates, polyalkylated polynuclear arylsulfonates, methylene-linked condensation products of arylsulfonic acids and hydroxyarylsulfonic acids, sodium salts of dialkylsulfosuccinic acid, sodium salts of alkyl diglycol ether sulfates, sodium salts of polynaphthalenemethanesulfonates, lignosulfonates or oxylignosulfonates and heterocyclic polysulfonic acids.

Especially suitable anionic dispersants are condensation products of naphthalenesulfonic acids with formaldehyde, sodium salts of polymerised organic sulfonic acids, (mono-/di-)-alkylnaphthalenesulfonates, polyalkylated polynuclear arylsulfonates, sodium salts of polymerised alkylbenzenesulfonic acid, lignosulfonates, oxylignosulfonates and condensation products of naphthalenesulfonic acid with a polychloromethyldiphenyl.

Suitable non-ionic dispersants are especially compounds having a melting point of, preferably, at least 35° C. that are emulsifiable, dispersible or soluble in water, for example the following compounds:
1. fatty alcohols having from 8 to 22 carbon atoms, especially cetyl alcohol;
2. addition products of, preferably, from 2 to 80 mol of alkylene oxide, especially ethylene oxide, wherein some of the ethylene oxide units may have been replaced by substituted epoxides, such as styrene oxide and/or propylene oxide, with higher unsaturated or saturated monoalcohols, fatty acids, fatty amines or fatty amides having from 8 to 22 carbon atoms or with benzyl alcohols, phenyl phenols, benzyl phenols or alkyl phenols, the alkyl radicals of which have at least 4 carbon atoms;
3. alkylene oxide, especially propylene oxide, condensation products (block polymers);
4. ethylene oxide/propylene oxide adducts with diamines, especially ethylenediamine;
5. reaction products of a fatty acid having from 8 to 22 carbon atoms and a primary or secondary amine having at least one hydroxy-lower alkyl or lower alkoxy-lower alkyl group, or alkylene oxide addition products of such hydroxyalkyl-group-containing reaction products;
6. sorbitan esters, preferably having long-chain ester groups, or ethoxylated sorbitan esters, such as polyoxyethylene sorbitan monolaurate having from 4 to 10 ethylene oxide units or polyoxyethylene sorbitan trioleate having from 4 to 20 ethylene oxide units;
7. addition products of propylene oxide with a tri- to hexa-hydric aliphatic alcohol having from 3 to 6 carbon atoms, e.g. glycerol or pentaerythritol; and
8. fatty alcohol polyglycol mixed ethers, especially addition products of from 3 to 30 mol of ethylene oxide and from 3 to 30 mol of propylene oxide with aliphatic monoalcohols having from 8 to 22 carbon atoms.

Especially suitable non-ionic dispersants are surfactants of formula

R₂₃—O-(alkylene-O)ₙ—R₂₄ (7),

wherein
R₂₃ is C₈-C₂₂alkyl or C₈-C₁₈alkenyl;
R₂₄ is hydrogen; C₁-C₄alkyl; a cycloaliphatic radical having at least 6 carbon atoms; or benzyl;
“alkylene” is an alkylene radical having from 2 to 4 carbon atoms and
n is a number from 1 to 60.

The substituents R₂₃ and R₂₄ in formula (7) are advantageously each the hydrocarbon radical of an unsaturated or, preferably, saturated aliphatic monoalcohol having from 8 to 22 carbon atoms. The hydrocarbon radical may be straight-chain or branched. R₂₃ and R₂₄ are preferably each independently of the other an alkyl radical having from 9 to 14 carbon atoms.

Aliphatic saturated monoalcohols that come into consideration include natural alcohols, e.g. lauryl alcohol, myristyl alcohol, cetyl alcohol or stearyl alcohol, and also synthetic alcohols, e.g. 2-ethylhexanol, 1,1,3,3-tetramethylbutanol, octan-2-ol, isononyl alcohol, trimethylhexanoyl, trimethylnonyl alcohol, decanoyl, C₉-C₁₁oxo-alcohol, tridecyl alcohol, isotridecyl alcohol and linear primary alcohols (Alfols) having from 8 to 22 carbon atoms. Some examples of such Alfols are Alfol (8-10), Alfol (9-11), Alfol (10-14), Alfol (12-13) and Alfol (16-18). (“Alfol” is a registered trade mark of the company Sasol Limited). Unsaturated aliphatic monoalcohols are, for example, dodecenyl alcohol, hexadecenyl alcohol and oleyl alcohol.

The alcohol radicals may be present singly or in the form of mixtures of two or more components, e.g. mixtures of alkyl and/or alkenyl groups that are derived from soybean fatty acids, palm kernel fatty acids or tallow oils.

(Alkylene-O) chains are preferably bivalent radicals of the formulae

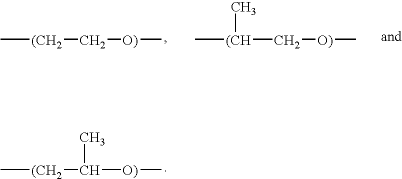

Examples of a cycloaliphatic radical include cycloheptyl, cyclooctyl and preferably cyclohexyl.

As non-ionic dispersants there come into consideration preferably surfactants of formula

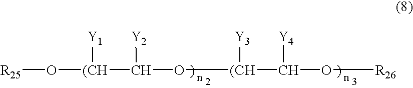

wherein
R₂₅ is C₈-C₂₂alkyl;
R₂₆ is hydrogen or C₁-C₄alkyl;
Y₁, Y₂, Y₃ and Y₄ are each independently of the others hydrogen, methyl or ethyl;
n₂ is a number from 0 to 8; and
n₃ is a number from 2 to 40.

Further important non-ionic dispersants correspond to formula

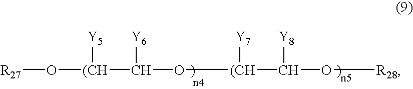

wherein
R₂₇ is C₉-C₁₄alkyl;
R₂₈ is C₁-C₄alkyl;
Y₅, Y₆, Y₇ and Y₈ are each independently of the others hydrogen, methyl or ethyl, one of the radicals Y₅, Y₆ and one of the radicals Y₇, Y₈ always being hydrogen; and
n₄ and n₅ are each independently of the other an integer from 4 to 8.

The non-ionic dispersants of formulae (7) to (9) can be used in the form of mixtures. For example, as surfactant mixtures there come into consideration non-end-group-terminated fatty alcohol ethoxylates of formula (7), e.g. compounds of formula (7) wherein

R₂₃ is C₈-C₂₂alkyl,
R₂₄ is hydrogen and
the alkylene-O chain is the radical —(CH₂—CH₂—O)— and
also end-group-terminated fatty alcohol ethoxylates of formula (9).

Examples of non-ionic dispersants of formulae (7), (8) and (9) include reaction products of a C₁₀-C₁₃fatty alcohol, e.g. a C₁₃oxo-alcohol, with from 3 to 10 mol of ethylene oxide, propylene oxide and/or butylene oxide and the reaction product of one mol of a C₁₃fatty alcohol with 6 mol of ethylene oxide and 1 mol of butylene oxide, it being possible for the addition products each to be end-group-terminated with C₁-C₄alkyl, preferably methyl or butyl.

Such dispersants can be used singly or in the form of mixtures of two or more dispersants. Instead of, or in addition to, the anionic or non-ionic dispersant, the granules according to the invention may comprise a water-soluble organic polymer as binder. Such polymers may be used singly or in the form of mixtures of two or more polymers.

Water-soluble polymers that come into consideration are, for example, polyethylene glycols, copolymers of ethylene oxide with propylene oxide, gelatin, polyacrylates, polymethacrylates, polyvinyl pyrrolidones, vinyl pyrrolidones, vinyl acetates, polyvinyl imidazoles, polyvinylpyridine-N-oxides, copolymers of vinylpyrrolidone with long-chain α-olefins, copolymers of vinylpyrrolidone with vinyl imidazole, poly(vinyl pyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyacrylamides, carboxymethyl cellulose, hydroxymethyl cellulose, polyvinyl alcohols, polyvinyl acetate, hydrolysed polyvinyl acetate, copolymers of ethyl acrylate with methacrylate and methacrylic acid, copolymers of maleic acid with unsaturated hydrocarbons, and also mixed polymerisation products of the mentioned polymers.

Of those organic polymers, special preference is given to polyethylene glycols, carboxymethyl cellulose, polyacrylamides, polyvinyl alcohols, polyvinylpyrrolidones, gelatin, hydrolysed polyvinyl acetates, copolymers of vinylpyrrolidone and vinyl acetate, and also polyacrylates, copolymers of ethyl acrylate with methacrylate and methacrylic acid, and polymethacrylates.

Suitable water-emulsifiable or water-dispersible binders also include paraffin waxes.

Encapsulating materials (c) include especially water-soluble and water-dispersible polymers and waxes. Of those materials, preference is given to polyethylene glycols, polyamides, polyacrylamides, polyvinyl alcohols, polyvinylpyrrolidones, gelatin, hydrolysed polyvinyl acetates, copolymers of vinylpyrrolidone and vinyl acetate, and also polyacrylates, paraffins, fatty acids, copolymers of ethyl acrylate with methacrylate and methacrylic acid, and polymethacrylates.

Further additives (d) that come into consideration are, for example, wetting agents, dust removers, water-insoluble or water-soluble dyes or pigments, and also dissolution accelerators, optical brighteners and sequestering agents.

The preparation of the granules according to the invention is carried out, for example, starting from:

a) a solution or suspension with a subsequent drying/shaping step or

b) a suspension of the active ingredient in a melt with subsequent shaping and solidification.

a) First of all the anionic or non-ionic dispersant and/or the polymer and, optionally, the further additives are dissolved in water and stirred, if desired with heating, until a homogeneous solution is obtained. The catalyst according to the invention is then dissolved or suspended in the resulting aqueous solution. The solids content of the solution should preferably be at least 30 wt-%, especially from 40 wt-% to 50 wt-%, based on the total weight of the solution. The viscosity of the solution is preferably less than 200 mPas.

The aqueous solution so prepared, comprising the catalyst according to the invention, is then subjected to a drying step in which all water, with the exception of a residual amount, is removed, solid particles (granules) being formed at the same time. Known methods are suitable for producing the granules from the aqueous solution. In principle, both continuous methods and discontinuous methods are suitable. Continuous methods are preferred, especially spray-drying and fluidised bed granulation processes.

Especially suitable are spray-drying processes in which the active ingredient solution is sprayed into a chamber with circulating hot air. The atomization of the solution is effected e.g. using unitary or binary nozzles or is brought about by the spinning effect of a rapidly rotating disc. In order to increase the particle size, the spray-drying process may be combined with an additional agglomeration of the liquid particles with solid nuclei in a fluidised bed that forms an integral part of the chamber (so-called fluid spray). The fine particles (<100 μm) obtained by a conventional spray-drying process may, if necessary after being separated from the exhaust gas flow, be fed as nuclei, without further treatment, directly into the atomizing cone of the atomiser of the spray-dryer for the purpose of agglomeration with the liquid droplets of the active ingredient.

During the granulation step, the water can rapidly be removed from the solutions comprising the catalyst according to the invention, binder and further additives. It is expressly intended that agglomeration of the droplets forming in the atomizing cone, or agglomeration of droplets with solid particles, will take place.

If necessary, the granules formed in the spray-dryer are removed in a continuous process, for example by a sieving operation. The fines and the oversize particles are either recycled directly to the process (without being redissolved) or are dissolved in the liquid active ingredient formulation and subsequently granulated again.

A further preparation method according to a) is a process in which the polymer is mixed with water and then the catalyst is dissolved/suspended in the polymer solution, thus forming an aqueous phase, the catalyst according to the invention being homogeneously distributed in that phase. At the same time or subsequently, the aqueous phase is dispersed in a water-immiscible liquid in the presence of a dispersion stabiliser in order that a stable dispersion is formed. The water is then removed from the dispersion by distillation, forming substantially dry particles. In those particles, the catalyst is homogeneously distributed in the polymer matrix.

The granules according to the invention are resistant to abrasion, low in dust, pourable and readily meterable. They can be added directly to a formulation, such as a detergent formulation, in the desired concentration of the catalyst according to the invention.

Where the coloured appearance of the granules in the detergent is to be suppressed, this can be achieved, for example, by embedding the granules in a droplet of a whitish meltable substance (“water-soluble wax”) or by adding a white pigment (e.g. TiO₂) to the granule formulation or, preferably, by encapsulating the granules in a melt consisting, for example, of a water-soluble wax, as described in EP-A-0 323 407, a white solid being added to the melt in order to reinforce the masking effect of the capsule.

b) The catalyst according to the invention is dried in a separate step prior to the melt-granulation and, if necessary, dry-ground in a mill so that all the solids particles are <50 μm in size. The drying is carried out in an apparatus customary for the purpose, for example in a paddle dryer, vacuum cabinet or freeze-dryer.

The finely particulate catalyst is suspended in the molten carrier material and homogenised. The desired granules are produced from the suspension in a shaping step with simultaneous solidification of the melt. The choice of a suitable melt-granulation process is made in accordance with the desired size of granules. In principle, any process which can be used to produce granules in a particle size of from 0.1 to 4 mm is suitable. Such processes are droplet processes (with solidification on a cooling belt or during free fall in cold air), melt-prilling (cooling medium gas/liquid), and flake formation with a subsequent comminution step, the granulation apparatus being operated continuously or discontinuously.

Where the coloured appearance of the granules prepared from a melt is to be suppressed in the detergent, in addition to the catalyst it is also possible to suspend in the melt white or coloured pigments which, after solidification, impart the desired coloured appearance to the granules (e.g. titanium dioxide).

If desired, the granules can be covered with or encapsulated in an encapsulating material. Methods that come into consideration for such an encapsulation include the customary methods and also encapsulation of the granules by a melt consisting e.g. of a water-soluble wax, as described, for example, in EP-A-0 323 407, coacervation, complex coacervation and surface polymerisation.

Encapsulating materials (c) include e.g. water-soluble, water-dispersible or water-emulsifiable polymers and waxes.

As further additives (d) there come into consideration, for example, wetting agents, dust removers, water-insoluble or water-soluble dyes or pigments, and also dissolution accelerators, optical brighteners and sequestering agents.

Other product forms of the present invention include product forms specifically developed for industrial and institutional cleaning, for example liquid solutions of the catalyst in water or organic solvents or solid forms such as powders or granules which can be dosed in a separate bleaching step of the cleaning application.

Surprisingly, the metal complex compounds of formula (1) also exhibit a markedly improved bleach-catalysing action on coloured stains occurring on kitchen surfaces, wall tiles or floor tiles.

The use of at least one metal complex compound of formula (1) as catalyst(s) in cleaning solutions for hard surfaces, especially for kitchen surfaces, wall tiles or floor tiles, is therefore of special interest.

The metal complex compounds of formula (1) and the corresponding ligands also have excellent antibacterial action. The use thereof for killing bacteria or for protecting against bacterial attack is therefore likewise of interest.

The invention also relates to new metal complexes of formula (1)

[LₙMeₘXₚ]^{z}Y_{q} (1),

wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2)

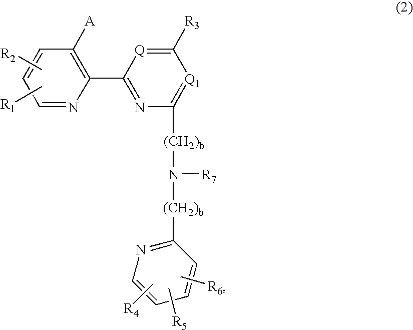

wherein
R₁, R₂, R₃, R₄, R₅, R₆ and R₇ are each independently of the others hydrogen; unsubstituted or substituted C₁-C₁₈alkyl or unsubstituted or substituted aryl; cyano; halogen; nitro; —COOR₉ or —SO₃R₉ wherein
R₉ is in each case hydrogen, a cation or unsubstituted or substituted C₁-C₁₈alkyl or unsubstituted or substituted aryl;
   —SR₁₀, —SO₂R₁₀ or —OR₁₀ wherein
R₁₀ is in each case hydrogen or unsubstituted or substituted C₁-C₁₈alkyl or unsubstituted or substituted aryl;
   —NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃;
   —N(R₁₀)—(C₁-C₆alkylene)-NR₁₁R₁₂; —N[(C₁-C₆alkylene)-NR₁₁R₁₂]₂;
   —N(R₁₀)—(C₁-C₆alkylene)-N^{⊕}R₁₁R₁₂R₁₃; —N[(C₁-C₆alkylene)-N^{⊕}R₁₁R₁₂R₁₃]₂; —N(R₁₀)—N—R₁₁R₁₂ or
   —N(R₁₀)—N^{⊕}R₁₁R₁₂R₁₃, wherein
R₁₀ is as defined above and
R₁₁, R₁₂ and R₁₃ are each independently of the other(s) hydrogen or unsubstituted or substituted C₁-C₁₈alkyl or unsubstituted or substituted aryl, or
R₁₁, and R₁₂, together with the nitrogen atom linking them, form an unsubstituted or substituted 5-, 6- or 7-membered ring which may contain further hetero atoms,
   Q is N or CR₈, wherein R₈ has the meanings as defined for R₁-R₇ or
R₈ forms together with A a

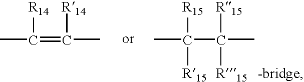

wherein R₁₄, R′₁₄, R₁₅, R′₁₅, R′₁₅ and R′″₁₅ independently from each other are H, C₁-C₄-alkyl or C₁-C₄-alkoxy,
   Q₁ is N or CR′₈, wherein R′₈ has the meanings as defined for R₁-R₇,
   A has one of the meanings as defined for R₁-R₇, or
A forms together with R₈ a

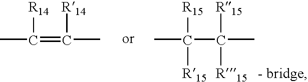

wherein R₁₄, R′₁₄, R₁₅, R′₁₅, R″₁₅ and R′″₁₅ have the same meanings as defined above b and c are each independently from each other 1, 2 or 3.

All preferences as mentioned above (for the use) apply also for the metal complex as such.

A preferred embodiment of the present invention also relates to new metal complex of formula (1′),

[L′ₙMe′_{m′}X′_{p′}]^{z}Y′_{q} (1′)

wherein
Me′is manganese, titanium, iron, cobalt, nickel or copper,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of from 4− to 4+, preferably from 0 to 4+, especially preferably the number 0,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer from 0 to 4, preferably the number 0,
L′ is a ligand of formula (2a), (2b) or (2d)

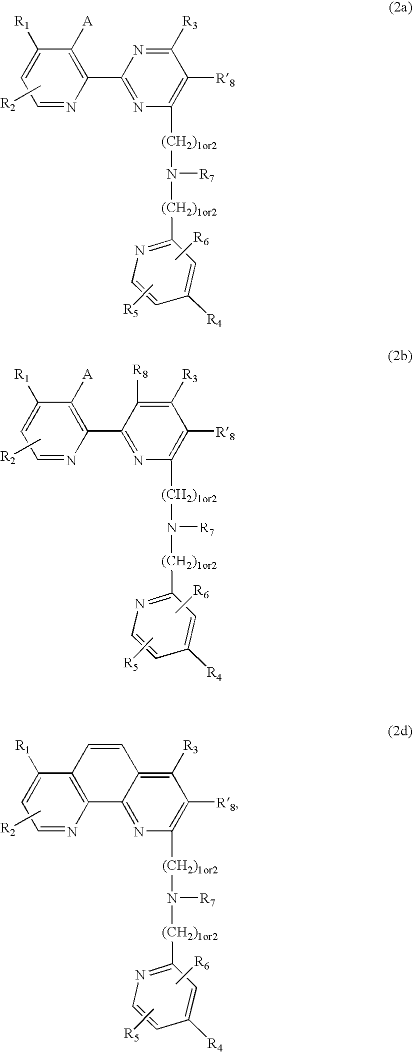

wherein
R₁, R₂, R₄, R₄, R₅, R₆, R₇, R₈, R′₈ and A are independently from each other hydrogen; unsubstituted C₁-C₁₂alkyl; C₁-C₁₂alkyl, which is substituted by at least one substituent chosen from the group consisting of —OH, —CN, —NH₂, COOH and COOC₁-C₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, phenyl, phenoxy or by naphthyloxy; cyano; halogen; nitro; —COOR₉ or —SO₃R₉ wherein R₉ is in each case hydrogen, a cation, C₁-C₁₂alkyl, unsubstituted phenyl or phenyl substituted as indicated above; —SR₁₀, —SO₂R₁₀ or —OR₁₀ wherein R₁₀ is in each case hydrogen, C₁-C₁₂alkyl, unsubstituted phenyl or phenyl substituted as indicated above;
—NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃; —N(R₁₀)—(C₁-C₆alkylene)-NR₁₁R₁₂; —N(R₁₀)—(C₁-C₆alkylene)-N^{⊕}R₁₁R₁₂R₁₃, —N(R₁₀)—N—R₁₁R₁₂ or —N(R₁₀)—N^{⊕}R₁₁R₁₂R₁₃, wherein R₁₀ is hydrogen, C₁-C₄alkyl or phenyl, and R₁₁, R₁₂ and R₁₃ are each independently of the other(s) hydrogen, unsubstituted or hydroxy-substituted C₁-C₁₂alkyl, unsubstituted phenyl or phenyl substituted as indicated above, or R₁₁ and R₁₂, together with the nitrogen atom linking them, form a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised.

An especially preferred embodiment of the present invention relates to new metal complex of formula (1′),

[L′ₙMe′_{m′}X′_{p′}]^{z}Y′_{q} (1′)

wherein
Me′ is manganese or iron,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of from 0 to 4+, preferably the number 0,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br, I⁻, citrate, oxalate or tartrate, wherein R₁₇ is hydrogen; C₁-C₄alkyl; phenyl, or sulfophenyl,
q′ is an integer from 0 to 4, preferably the number 0,
L′ is a ligand of formula (2′a), (2′b) or (2′d)

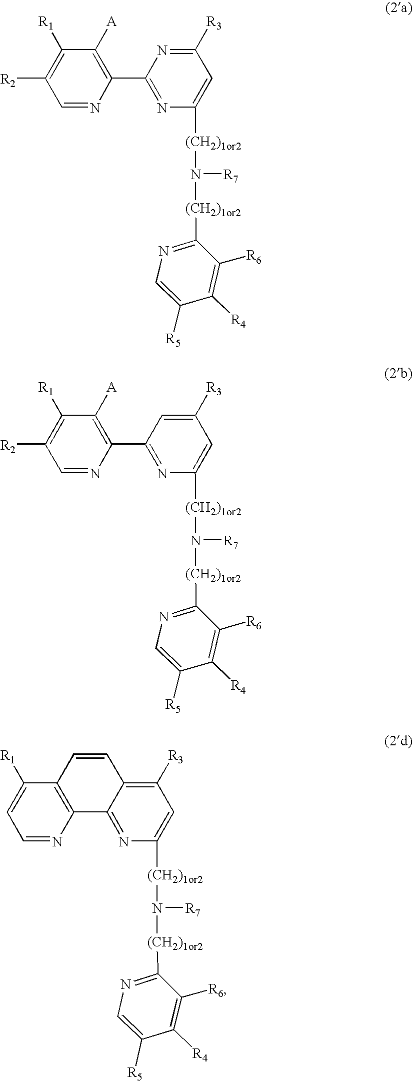

wherein
R₁ and R₄, are independently from each other H; —CH₃; —Cl; —OH; —OCH₃; —NH₂; —N(CH₃)₂;

### —N(CH₂CH₃)₂; —N(CH₃)(CH₂CH₃);

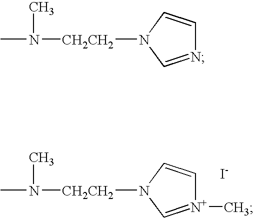

—N(CH₂CH₂OH)₂; —N(CH₂CH₃)(CH₂CH₂OH); —N(CH₃)CH₂CH₂OH; —N(CH₃)CH₂CH₂NH₂;

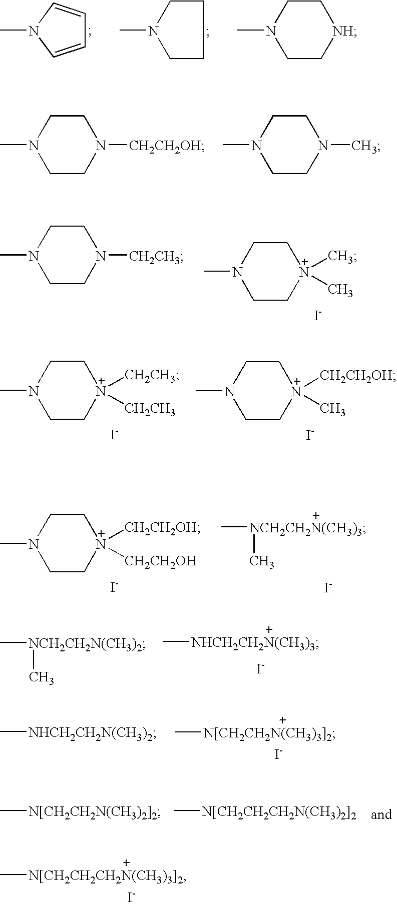

A and R₂, are independently from each other H or —CH₃,

### R₃ is —OH; —OCH₃; —NH₂; —N(CH₃)₂; —N(CH₂CH₃)₂; —N(CH₃)(CH₂CH₃);

—N(CH₂CH₂OH)₂; —N(CH₂CH₃)(CH₂CH₂OH); —N(CH₃)CH₂CH₂OH;

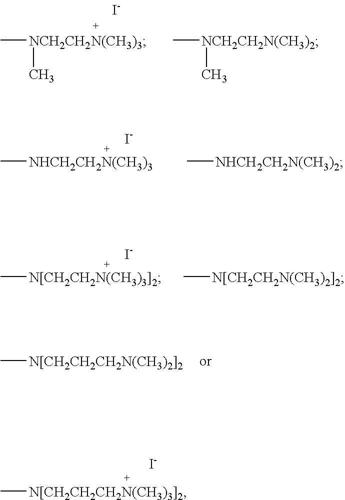

R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and

### R₇ is H; —CH₃; —CH₂COOH; —CH₂CH₂COOH; —CH₂CN or —CH₂CH₂CN.

The invention also related to new ligands of formula (2)

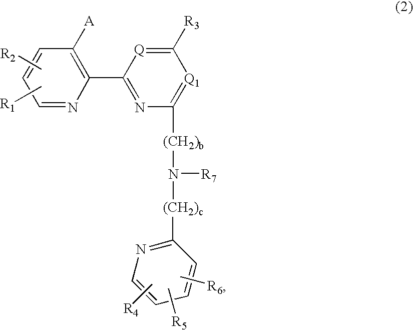

wherein
R₁, R₂, R₃, R₄, R₅, R₆ and R₇ are each independently of the others hydrogen; unsubstituted or substituted C₁-C₁₈alkyl or unsubstituted or substituted aryl; cyano; halogen; nitro; —COOR₉ or —SO₃R₉ wherein
R₉ is in each case hydrogen, a cation or unsubstituted or substituted C₁-C₁₈alkyl or unsubstituted or substituted aryl;
   —SR₁₀, —SO₂R₁₀ or —OR₁₀ wherein
R₁₀ is in each case hydrogen or unsubstituted or substituted C₁-C₁₈alkyl or unsubstituted or substituted aryl;
   —NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃;
   —N(R₁₀)—(C₁-C₆alkylene)-NR₁₁R₁₂; —N[(C₁-C₆alkylene)-NR₁₁R₁₂]₂;
   —N(R₁₀)—(C₁-C₆alkylene)-N^{⊕}R₁₁R₁₂R₁₃; —N[(C₁-C₆alkylene)-N^{⊕}R₁₁R₁₂R₁₃]₂; —N(R₁₀)—N—R₁₁R₁₂ or
   —N(R₁₀)—N^{⊕}R₁₁R₁₂R₁₃, wherein
R₁₀ is as defined above and
R₁₁, R₁₂ and R₁₃ are each independently of the other(s) hydrogen or unsubstituted or substituted C₁-C₁₈alkyl or unsubstituted or substituted aryl, or
R₁₁ and R₁₂, together with the nitrogen atom linking them, form an unsubstituted or substituted 5-, 6- or 7-membered ring which may contain further hetero atoms,
   Q is N or CR₈, wherein R₈ has the meanings as defined for R₁-R₇ or
R₈ forms together with A a

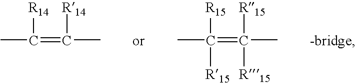

wherein R₁₄, R′₁₄, R₁₅, R′₁₅, R″₁₅ and R′″₁₅ independently from each other are H. C₁-C₄-alkyl or C₁-C₄-alkoxy,
   Q₁ is N or CR′₈, wherein R′₈ has the meanings as defined for R₁-R₇,
   A has one of the meanings as defined for R₁-R₇, or
A forms together with R₈ a

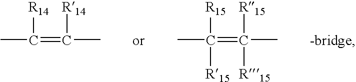

wherein R₁₄, R′₁₄, R₁₅, R′₁₅, R″₁₅ and R′″₁₅ have the same meanings as defined above b and c are each independently from each other 1, 2 or 3.

All preferences as mentioned above (for the use and the metal complexes) apply also for the metal complex as such.

A preferred embodiment of the present invention also relates to new ligands (2a), (2b) or (2d)

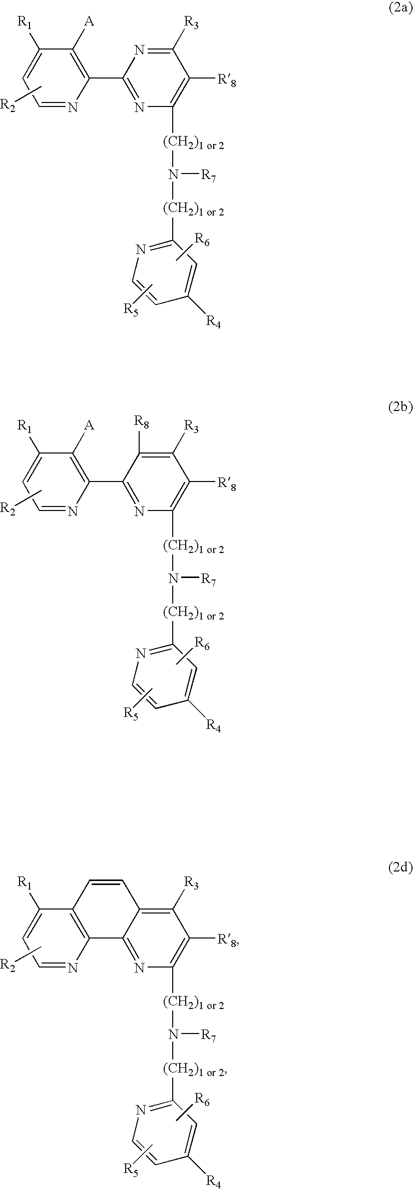

wherein
R₁, R₂, R₄, R₄, R₅, R₆, R₇, R₈, R′₈ and A are independently from each other hydrogen; unsubstituted C₁-C₁₂alkyl; C₁-C₁₂alkyl, which is substituted by at least one substituent chosen from the group consisting of —OH, —CN, —NH₂, COOH and COOC₁-C₂alkyl; phenyl unsubstituted or substituted by C₁-C₄alkyl, C₁-C₄alkoxy, halogen, cyano, nitro, carboxy, sulfo, hydroxy, amino, N-mono- or N,N-di-C₁-C₄alkylamino unsubstituted or substituted by hydroxy in the alkyl moiety, N-phenylamino, N-naphthylamino, phenyl, phenoxy or by naphthyloxy; cyano; halogen; nitro; —COOR₉ or —SO₃R₉ wherein R₉ is in each case hydrogen, a cation, C₁-C₁₂alkyl, unsubstituted phenyl or phenyl substituted as indicated above; —SR₁₀, —SO₂R₁₀ or —OR₁₀ wherein R₁₀ is in each case hydrogen, C₁-C₁₂alkyl, unsubstituted phenyl or phenyl substituted as indicated above;
—NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃; —N(R₁₀)—(C₁-C₆alkylene)-NR₁₁R₁₂; —N(R₁₀)—(C₁-C₆alkylene)-N^{⊕}R₁₁R₁₂R₁₃, —N(R₁₀)—N—R₁₁R₁₂ or —N(R₁₀)—N^{⊕}R₁₁R₁₂R₁₃, wherein R₁₀ is hydrogen, C₁-C₄alkyl or phenyl, and R₁₁, R₁₂ and R₁₃ are each independently of the other(s) hydrogen, unsubstituted or hydroxy-substituted C₁-C₁₂alkyl, unsubstituted phenyl or phenyl substituted as indicated above, or R₁₁ and R₁₂, together with the nitrogen atom linking them, form a imidazole, pyrazole, pyrrolidine, piperidine, piperazine, morpholine or azepane ring unsubstituted or substituted by at least one unsubstituted C₁-C₄alkyl and/or substituted C₁-C₄alkyl, wherein the nitrogen atom may be quaternised.

An especially preferred embodiment of the present invention relates to new ligands of formula (2′a), (2′b) and (2′d)

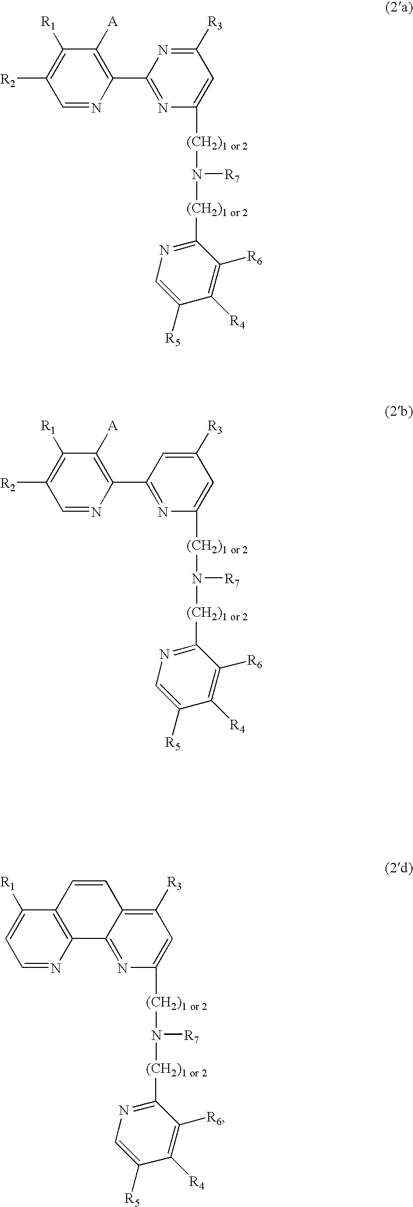

wherein
R₁ and R₄, are independently from each other H; —CH₃; —Cl; —OH; —OCH₃; —NH₂; —N(CH₃)₂;

### —N(CH₂CH₃)₂; —N(CH₃)(CH₂CH₃);

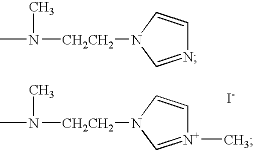

—N(CH₂CH₂OH)₂; —N(CH₂CH₃)(CH₂CH₂OH); —N(CH₃)CH₂CH₂OH; —N(CH₃)CH₂CH₂NH₂;

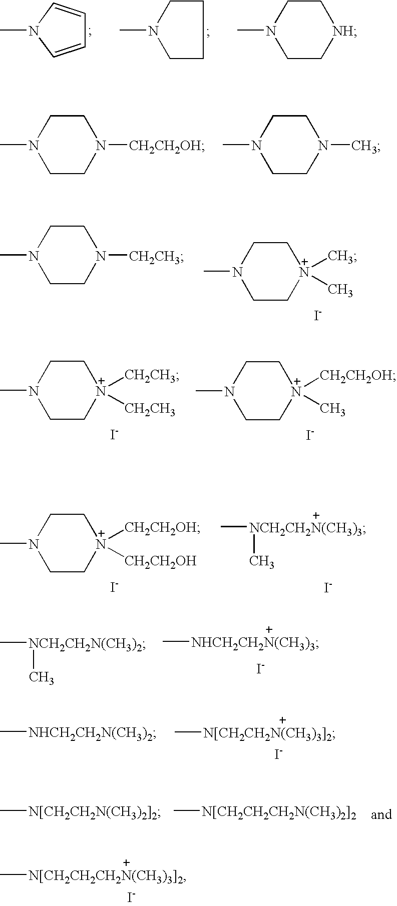

A and R₂, are independently from each other H or —CH₃,

### R₃ is —OH; —OCH₃; —NH₂; —N(CH₃)₂; —N(CH₂CH₃)₂; —N(CH₃)(CH₂CH₃);

—N(CH₂CH₂OH)₂; —N(CH₂CH₃)(CH₂CH₂OH); —N(CH₃)CH₂CH₂OH;

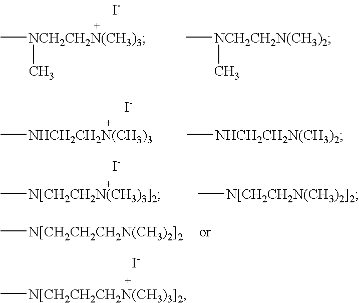

R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and

### R₇ is H; —CH₃; —CH₂COOH; —CH₂CH₂COOH; —CH₂CN or —CH₂CH₂CN.

Another embodiment of the present invention is the process of production of compounds of formula (2). A suitable process is for example a condensation reaction according to the following reaction scheme:

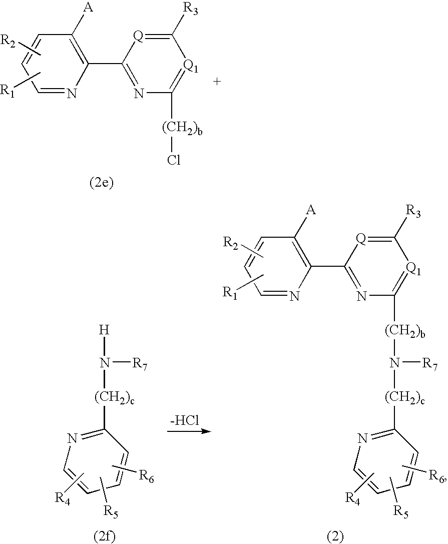

wherein all the substituents have the meanings as defined above.

The starting materials are known or can be produced according to known processes. The metal complexes of formula (1) are produced according to commonly processes. A suitable way is to react the ligands of formula (2) with a suitable metall salt at desired molar ratio.

The reaction of compound (2e) and (2f) is carried out in suitable solvents such as THF in the present of a base such as triethylamine. The reaction temperature is usually between 20° C. and 180° C.

The following Examples serve to illustrate the invention but do not limit the invention thereto. Parts and percentages relate to weight, unless otherwise indicated. Temperatures are in degrees Celsius, unless otherwise indicated.

### EXAMPLES

### Synthesis of Secondary Amine Building Blocks

### Example 1

### methyl-pyridin-2-ylmethyl-amine

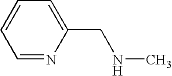

To an aqueous methylamine solution (300 ml, 40% w/w) in isopropanol (300 ml) was added 2-chloromethyl-pyridine hydrochloride (8 g, 48.8 mmol). The solution was stirred at room temperature for two days. After evaporation, aqueous carbonate solution (80 ml) was added, and the aqueous layer was extracted with dichloromethane (4×100 ml each). The combined organic phases were dried over sodium sulfate, filtered and evaporated. After distillation in a Kugelrohr apparatus, methyl-pyridin-2-ylmethyl-amine was obtained as a yellowish oil.

C₇H₁₀N₂, F_{w} 122.17.

¹H NMR (360 MHz, CDCl₃): δ=8.39 (d, J=4.5 Hz, 1H); 7.48 (ddd, J=7.7 Hz, 7.7 Hz, 1.8 Hz, 1H); 7.13 (d, J=7.7, 1H); 7.20-6.94 (m, 1H); 3.67 (s, 2H); 2.31 (s, 3H); 1.72 (br s, 1H).

### Example 2

### (4-methoxy-3,5-dimethyl-pyridin-2-ylmethyl)-methyl-amine

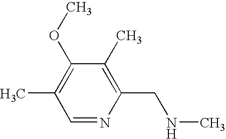

To a solution of 2-chloromethyl-3,5-dimethyl-4-methoxypyridine hydrochloride (6.0 g, 27 mmol) in isopropanol (180 ml) was added aqueous methylamine solution (180 ml, 40% w/w), and the mixture was stirred for two days at room temperature. After evaporation, aqueous carbonate solution (40 ml) was added, and the aqueous layer was extracted with dichloromethane (5×100 ml each). The combined organic phases were dried over sodium sulfate, filtered and evaporated to give (4-methoxy-3,5-dimethyl-pyridin-2-ylmethyl)-methyl-amine as a yellowish solid.

C₁₀H₁₆N₂O, F_{w} 180.25.

¹H NMR (360 MHz, CDCl₃): δ=8.03 (s, 1H); 3.64 (s, 2H); 3.59 (s, 3H); 2.36 (s, 3H); 2.08 (s, 6H); 1.91 (br s, 1H).

### Example 3

### ethyl 4-chloropicolinate

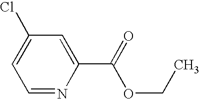

N,N′-Dimethylformamide (10.0 ml, 130 mmol) was cautiously added with stirring to thionyl chloride (295 ml, 4.06 mol) at 40° C. After 30 min, finely powdered picolinic acid (100 g, 812 mmol) was added in 10 equal portions over 30 min, while keeping the temperature between 38 and 42° C. The temperature was raised to 70° C. over 2 h (vigorous evolution of SO₂/HCl), and the mixture was stirred at this temperature for 1 day. Part of the volatiles (ca. 150 ml) was distilled off and replaced by toluene (ca. 150 ml), and the removal of volatiles (again ca. 150 ml) was repeated once more. Toluene was then added to a total volume of about 450 ml, and the resulting solution was poured into an ethanol-toluene solution (1:1 (v/v), 240 ml) cooled in an ice-bath. The resulting suspension was stirred overnight and concentrated to half of the volume on a rotary evaporator. The mixture was filtered at 0° C. and washed with toluene (250 ml). After drying in vacuo, ethyl 4-chloropicolinate hydrochloride was obtained as a beige solid.

The above hydrochloride was partitioned between ethyl acetate (300 ml) and water (200 ml), and quickly neutralized with aqueous sodium hydroxide (125 ml, 4 M). The aqueous layer was separated and extracted twice with ethyl acetate (200 ml each). The combined organic layers were dried over sodium sulfate (50 g), filtered and concentrated to dryness. After distillation in vacuo (0.2 mbar, 65-70° C.), ethyl 4-chloropicolinate was obtained as a colorless semisolid.

C₈H₈ClNO₂, F_{w} 185.61.

¹H NMR (360 MHz, CDCl₃): δ=8.56 (d, J=5.0 Hz, 1H); 8.03 (d, J=1.8 Hz, 1H); 7.43-7.37 (m, 1H); 4.39 (q, J=7.2 Hz, 2H); 1.35 (t, J=7.2 Hz, 3H).

### Example 4

### (4-chloro-pyridin-2-yl)-methanol

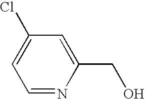

To a solution of ethyl 4-chloropicolinate (11.14 g, 60 mmol) in abs. ethanol (450 ml) was added, with stirring at 0° C., sodium borohydride (3.63 g, 96 mmol). The mixture was stirred at for one hour, and another 30 minutes at room temperature. After heating to 70° C. for one hour, water was added cautiously at room temperature. The pH-value was adjusted to 5.5 with aqueous HCl (4M). After stirring for 14 hours, the volatiles were removed in vacuo. Water was added, and the mixture was extracted with dichloromethane (2×200 ml each). The organic phase was dried over sodium sulfate, filtered, and evaporated to yield (4-chloro-pyridin-2-yl)-methanol as a clear yellow oil.

C₆H₆ClNO, F_{w} 143.57.

¹H NMR (360 MHz, CDCl₃): δ=8.33 (d, J=5.4 Hz, 1H); 7.28 (d, J=1.6 Hz, 1H); 7.12 (dd, J=5.4, 1.6 Hz, 1H); 4.66 (s, 2H); 3.98 (br s, 1H).

### Example 5

### 4-chloro-2-chloromethyl-pyridine

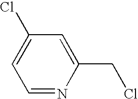

To a solution of (4-chloro-pyridin-2-yl)-methanol (7.70 g, 53.6 mmol) in dichloromethane (350 ml), thionyl chloride (8.16 g, 68.6 mmol) was added with stirring at room temperature. Water (30 ml) was added after one hour, and the pH-value was adjusted to 8. The aqueous layer was extracted with dichloromethane (2×150 ml each). The combined organic phases were dried over sodium sulfate, filtered and evaporated to yield 4-chloro-2-chloromethyl-pyridine as a yellowish oil.

C₆H₅Cl₂N, F_{w} 162.02.

¹H NMR (360 MHz, CDCl₃): δ=8.47 (d, J=5.4 Hz, 1H); 7.52 (d, J=1.8 Hz, 1H); 7.30-7.22 (m, 1H); 4.65 (s, 2H).

### Example 6

### (4-chloro-pyridin-2-ylmethyl)-methyl-amine

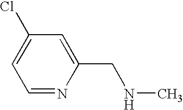

To a solution of 4-chloro-2-chloromethyl-pyridine (8.9 g, 55 mmol) in isopropanol (150 ml) was added aqueous methylamine (100 ml, 40% w/w), and the mixture was stirred for 16 hours at room temperature. After evaporation, the residue was partitioned between water and chloroform at pH=9. The combined organic extracts were dried over sodium sulfate, filtered, and evaporated to yield (4-chloro-pyridin-2-ylmethyl)-methyl-amine as a yellowish solid.

C₇H₉ClN₂, F_{w} 156.62.

¹H NMR (360 MHz, CDCl₃): δ=8.43 (d, J=5.4 Hz, 1H); 7.45 (d, J=1.8 Hz, 1H); 7.23-7.17 (m, 1H); 5.95 (br s, 1H); 4.11 (s, 2H); 2.61 (s, 3H).

### Example 7

### (4-dimethylamino-pyridin-2-yl)-methanol

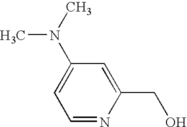

A solution of (4-chloro-pyridin-2-yl)-methanol (6.68 g, 47 mmol) in aqueous dimethylamine (30 ml, 7.9 M, 5 equiv.) was heated to 155° C. for 14 hours in a pressure reactor. After evaporation of the volatiles, the residue was taken up in water and the pH adjusted to a value of 8.5 with sodium hydroxide (4M in water). The mixture was concentrated on a rotary evaporator, and the beige solid was extracted with chloroform in a Soxhlet apparatus for 16 hours. The extract was evaporated, and the crude product was recrystallized from methanol/diethyl ether.

C₈H₁₂N₂O, F_{w} 152.20.

¹H NMR (360 MHz, CDCl₃): δ=7.88 (d, J=6.8 Hz, 1H); 6.76-6.60 (m, 2H); 4.67 (s, 2H); 3.12 (s, 6H).

### Example 8

### (2-chloromethyl-pyridin-4-yl)-dimethyl-amine

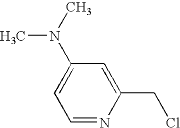

To a solution of (4-dimethylamino-pyridin-2-yl)-methanol (4.50 g, 29.6 mmol) in dichloromethane (350 ml), thionyl chloride (4.50 g, 37.9 mmol) was added at room temperature. Water (150 ml) was added after stirring for 1.5 hours, and the pH-value was adjusted to 8.5. The aqueous layer was extracted with dichloromethane (2×100 ml each). The combined organic phases were dried over sodium sulfate, filtered and evaporated to yield (2-chloromethyl-pyridin-4-yl)-dimethyl-amine as a brownish semisolid.

C₈H₁₁ClN₂, F_{w} 170.64.

¹H NMR (360 MHz, CDCl₃): δ=8.10 (d, J=5.8 Hz, 1H); 6.57 (d, J=2.6 Hz, 1H); 6.33 (dd, J=5.8, 2.6 Hz, 1H); 4.49 (s, 2H); 2.94 (s, 6H).

### Example 9

### dimethyl-(2-methylaminomethyl-pyridin-4-yl)-amine

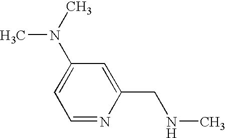

A mixture containing (2-chloromethyl-pyridin-4-yl)-dimethyl-amine (3.67 g, 21.5 mmol), aqueous methylamine (50 ml, 40% w/w), and isopropanol (100 ml) was stirred for 20 hours at room temperature. The volatiles were removed in vacuo, and dimethyl-(2-methylaminomethyl-pyridin-4-yl)-amine containing HCl was isolated as a beige solid.

C₉H₁₅N₃, F_{w} 165.24.

¹H NMR (360 MHz, CDCl₃): δ=8.05 (d, J=5.8 Hz, 1H); 6.74 (d, J=2.3 Hz, 1H); 6.38 (dd, J=5.8, 2.3 Hz, 1H); 6.17 (br s, >1H); 4.00 (s, 2H); 2.97 (s, 6H); 2.54 (s, 3H).

### Example 9a

### (4-pyrrol-1-yl-pyridin-2-yl)-methanol

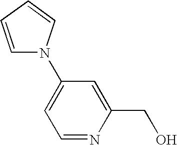

(4-Chloro-pyridin-2-yl)-methanol (1.71 g, 11.9 mmol) was suspended in 20 ml 1,3-Dimethyl-2-imidazolidinone (DMEU) under an inert atmosphere at 0° C. Pyrrol (1.97 ml, 28.4 mmol) and potassium tert.-butylate (3.2 g, 28.5 mmol) were added, and the mixture was stirred for 6 hours at 105° C. After cooling to room temperature, the reaction mixture was stirred for additional 12 hours at room temperature. After evaporation of the volatiles, the residue was partitioned between water and diethyl ether at pH 9. The combined organic extracts were dried over sodium sulfate, filtered, and evaporated to yield the crude product. After column chromatography (silica gel, hexane/ethyl acetate 1:2), (4-pyrrol-1-yl-pyridin-2-yl)-methanol was obtained as brownish powder.

C₁₀H₁₀N₂O, F_{w} 174.20.

¹H NMR (360 MHz, CDCl₃): δ=8.43 (d, J=5.5 Hz, 1H); 7.22 (s, 1H); 7.03-7.15 (m, 3H); 6.25-6.38 (m, 2H); 4.73 (s, 2H); 2.95 (s, br, 1H, OH).

### Example 9b

### 2-chloromethyl-4-pyrrol-1-yl-pyridine

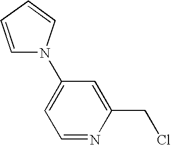

To a solution of (4-pyrrol-1-yl-pyridin-2-yl)-methanol (130 mg, 0.746 mmol) in dichloromethane (10 ml), thionyl chloride (70 μl, 0.955 mol) was added at room temperature. Water (10 ml) was added after stirring for 2 hours, and the pH-value was adjusted to 8.5. The aqueous layer was extracted with dichloromethane (2×50 ml). The combined organic phases were dried over sodium sulfate, filtered and evaporated to yield 2-chloromethyl-4-pyrrol-1-yl-pyridine as brownish oil. The crude product thus obtained was used in the next step without further purification.

C₁₀H₉ClN₂, F_{w} 192.65.

### Example 9c

### methyl-(4-pyrrol-1-yl-pyridin-2-ylmethyl)-amine

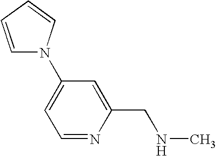

A mixture containing 2-chloromethyl-4-pyrrol-1-yl-pyridine (86 mmg, 0.446 mmol), aqueous methylamine (1.2 ml, 40% w/w), and isopropanol (3 ml) was stirred for 16 hours at room temperature. The volatiles were removed in vacuo, and the crude methyl-(4-pyrrol-1-yl-pyridin-2-ylmethyl)-amine was obtained as brown oil, which was used in the next step without purification.

C₁₁H₁₃N₃, F_{w} 187.25.

### Synthesis of Chloromethyl Building Blocks

### Example 10

### 6-chloromethyl-2-pyridin-2-yl-pyrimidin-4-ol

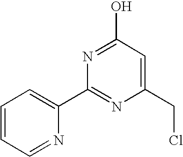

A mixture of pyridine-2-carboxamidine hydrochloride (3.15 g, 20 mmol), potassium t-butoxide (2.24 g, 20 mmol), and methyl 4-chloroacetoacetate (7.05 ml, 60 mmol) in ethanol (90 ml) was heated to reflux for 17 hours. After evaporation, the crude mixture was partitioned between chloroform and water (pH=7). The organic layer was dried over sodium sulfate, filtered and evaporated. The crude material was recrystallized from isopropanol to yield 6-chloromethyl-2-pyridin-2-yl-pyrimidin-4-ol as a white solid.

C₁₀H₈ClN₃O, F_{w} 221.65.

¹H NMR (360 MHz, CDCl₃): δ=10.97 (br s, 1H); 8.60 (d, J=4 Hz, 1H); 8.39 (d, J=8.1 Hz, 1H); 7.84 (tm, 1H); 7.44 (ddm, 1H); 6.58 (s, 1H); 4.40 (s, 2H).

### Example 11

### 6-chloromethyl-2-(4-chloro-pyridin-2-yl)-pyrimidin-4-ol

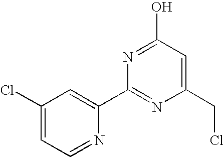

A solution of pyridine-2-carboxamidine hydrochloride (192 mg, 1 mmol) and DBU (298 μl, 2 mmol) in methanol (10 ml), was added to methyl 4-chloroacetoacetate (585 μl, 5 mmol) in methanol (20 ml) over a period of 1.5 hours at 60° C. The volatiles were removed in vacuo, and the mixture was taken up in water (pH=7). The crude product was isolated via filtration and recrystallized from isopropanol to yield 6-chloromethyl-2-(4-chloro-pyridin-2-yl)-pyrimidin-4-ol as a white solid.

C₁₀H₇Cl₂N₃O, F_{w} 256.09.

¹H NMR (360 MHz, CDCl₃): δ=10.83 (br s, 1H); 8.49 (d, J=5.4 Hz, 1H); 8.39 (d, J=1.8 Hz, 1H); 7.84 (dd, J=5.4 Hz, 1.8 Hz, 1H); 6.59 (s, 1H); 4.39 (s, 2H).

### Example 12

### 1-pyridin-2-yl-butane-1,3-dione

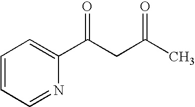

A solution of dry acetone (8.71 g, 150 mmol) in absolute tetrahydrofuran (100 ml) was added under argon to a solution of sodium ethoxide (20.42 g, 300 mmol) in absolute tetrahydrofuran (300 ml). A solution of ethyl pyridine-2-carboxylate (22.68 g, 150 mmol) in absolute tetrahydrofuran (100 ml) was subsequently added dropwise over the course of 20 minutes. The mixture was stirred for 15 hours at room temperature and for 4 hours at reflux. The mixture was evaporated on a rotary evaporator, admixed water (150 ml) and rendered neutral with glacial acetic acid. It was extracted twice with diethyl ether, and the organic extracts were combined and dried (sodium sulfate), evaporated on a rotary evaporator, and 1-pyridin-2-ylbutane-1,3-dione was obtained as an orange oil.

¹H NMR (360 MHz, CDCl₃) for enol tautomer: 15.8-15.5 (br s, OH); 8.60-8.55 (dm, 1H); 8.20-7.95 (dm, 1H); 7.79-7.71 (tm, 1H); 7.35-7.29 (m, 1H); 6.74 (s, 1H); 2.15 (s, 3H). Keto tautomer: CH₂ group at 4.20 ppm (ratio of enol/keto form=87:13).

### Example 13

### 2,4,6-trioxo-6-pyridin-2-yl-hexanoic acid ethyl ester

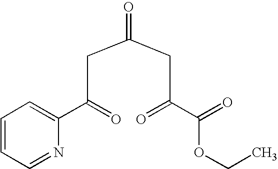

To a stirred suspension of sodium hydride (16.7 g, 60% pure, 417 mmol) in dry 1,2-dimethoxyethane (200 ml) was added over a period of 1 hour a solution of 1-pyridin-2-yl-butane-1,3-dione (22.7 g, 139 mmol) and diethyl oxalate (40.6 g, 278 mmol) in 100 ml 1,2-dimethoxyethane at reflux. The reaction mixture was refluxed for 3 hours, evaporated, and treated cautiously with hydrochloric acid (2M, 150 ml). After stirring for 10 minutes, the precipitate was filtered, washed with water and dried to give 2,4,6-trioxo-6-pyridin-2-yl-hexanoic acid ethyl ester as a yellow solid.

C₁₃H₁₃NO₅, F_{w} 263.25.

¹H NMR (360 MHz, CDCl₃): δ=14.3 (br s, 1H); 13.0 (br s, 1H); 8.8 (d, J=6.3 Hz, 1H); 8.2-7.9 (m, 2H); 7.6-7.4 (m, 1H); 6.9 (s, 1H); 6.45 (s, 1H); 4.45 (q, J=7.5 Hz, 2H); 1.4 (t, J=7.5 Hz, 3H).

### Example 14

### 4-hydroxy-[2,2′]bipyridinyl-6-carboxylic acid ethyl ester

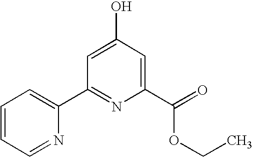

2,4,6-trioxo-6-pyridin-2-yl-hexanoic acid ethyl ester (12 g, 49 mmol) was suspended in ethanol (300 ml). After addition of ammonium acetate (25 g, 324 mmol), the mixture was heated to reflux for four hours. After evaporation of the volatiles, the residue was triturated with aq. sodium hydroxide solution to achieve pH=7. After extraction with chloroform (3×300 ml), the organic layers were combined, dried over sodium sulfate and evaporated. The crude material was purified by column chromatography on silica gel (chloroform/methanol/aq. ammonia 95:4:1) to give 4-hydroxy-[2,2′]bipyridinyl-6-carboxylic acid ethyl ester as a brownish solid.

C₁₃H₂, N₂O₃, F_{w} 244.25.

¹H NMR (360 MHz, CD₃OD): 6=¹H NMR (360 MHz, CDCl₃): δ=8.65 (d, J=4.5 Hz, 1H); 8.28 (d, J=7.7 Hz, 1H); 7.94 (ddd, J=7.7, 7.7, 1.4 Hz, 1H); 7.57 (br s, 1H); 7.50-7.44 (qm, 1H); 7.30 (br s, 1H); 4.86 (br s, 1H); 4.45 (q, J=6.8 Hz, 2H); 1.43 (t, J=6.8 Hz, 3H).

### Example 15

### 6-hydroxymethyl-[2,2′]bipyridinyl-4-ol

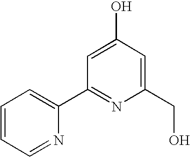

To a stirred suspension of lithium aluminium hydride (912 mg, 24 mmol) in dry tetrahydrofuran (30 ml) is added, at 0° C. over a period of 45 minutes, a solution of 4-hydroxy-[2,2′]bipyridinyl-6-carboxylic acid ethyl ester (1.46 g, 6 mmol) in dry tetrahydrofuran (20 ml) under argon. The mixture is stirred for two hours at room temperature. The resulting suspension is cooled to 0° C. and treated with dilute hydrochloric acid to achieve pH=7. After filtration through celite, the filtrate is evaporated, and the pure product is isolated by column chromatography (silica gel, chloroform/methanol/aq. ammonia 9:1:0.1) as a beige solid.

C₁₁H₁₀N₂O₂, F_{w} 202.21.

¹H NMR (360 MHz, CD₃OD): δ=8.72 (d, J=5.0 Hz, 1H); 8.10 (d, J=8.1 Hz, 1H); 7.97 (ddd, J=7.7, 7.7, 1.4 Hz, 1H); 7.58-7.48 (qm, 1H); 7.08 (d, J=2.3 Hz, 1H); 6.50 (s, 1H); 4.69 (s, 2H).

### Example 16

### 6-chloromethyl-[2,2′]bipyridinyl-4-ol

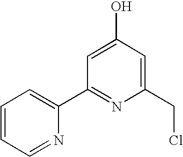

6-hydroxymethyl-[2,2]bipyridinyl-4-ol (100 mg, 0.5 mmol) was suspended in dry dichloromethane under an inert atmosphere at 0° C. Thionyl chloride (154 mg, 1.14 mmol, 2.28 equiv.) was added dropwise with stirring. After 15 minutes, the cooling bath was removed, and the mixture was stirred for 40 minutes at room temperature. After cooling to 0° C., water (10 ml) was added, and the pH value of the mixture was adjusted to 7 with sodium carbonate solution. The organic layer was separated, and the aqueous layer was extracted with dichloromethane (2×100 ml). The combined organic extracts were dried over sodium sulfate, filtered and evaporated at 30° C. to give 6-chloromethyl-[2,2′]bipyridinyl-4-ol as an off-white semisolid.

C₁₁H₉ClN₂O, F, 220.66.

¹H NMR (360 MHz, CDCl₃): δ=8.57 (d, J=4.0 Hz, 1H); 8.10 (d, J=7.7 Hz, 1H); 7.81-7.75 (tm, 1H); 7.33-7.29 (m, 2H); 6.69 (sm, 1H); 4.61 (s, 2H).

### Example 17

### 6-(4-chloro-pyridin-2-yl)-2,4,6-trioxo-hexanoic acid ethyl ester

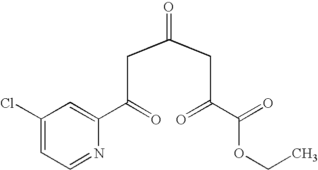

In a three-necked flask under argon, sodium hydride (60% in mineral oil, 10.0 g, 0.25 mol, 2.5 equiv.) was washed twice with n-hexane (100 ml each). Dry tetrahydrofuran (140 ml) was added, and the mixture was heated to 62° C. A solution of ethyl 4-chloropicolinate (18.6 g, 0.1 mol) and ethyl acetopyruvate (15.8 g, 0.1 mol) in dry tetrahydrofuran (30 ml) was added with stirring over a period of one hour. During addition, strong evolution of hydrogen gas indicated the progress of the reaction. The reddish suspension was stirred for another 15 minutes at 62° C., and subsequently cooled to 0° C. in an ice/water bath. Water (100 ml) was carefully added with. Aqueous HCl was added to the orange suspension (4M, ca. 50 ml; final pH value=7). The resulting yellow suspension was filtered and dried. The crude product thus obtained was used without further purification.

C₁₃H₁₂ClNO₅, F_{w} 297.70.

¹H NMR (360 MHz, CDCl₃, dienol tautomer): δ=14.7 (br s, 1H); 13.2 (br s, 1H); 8.60 (m, 1H); 8.05 (m, 1H); 7.35 (m, 1H); 6.80 (s, 1H); 6.35 (s, 1H); 4.37 (q, J=7.0 Hz, 2H); 1.28 (t, J=7.0 Hz, 3H).

### Example 18

### 4′-chloro-4-hydroxy-[2,2′]bipyridinyl-6-carboxylic acid ethyl ester

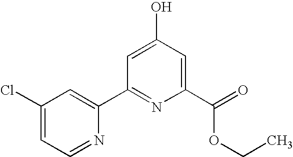

6-(4-Chloro-pyridin-2-yl)-2,4,6-trioxo-hexanoic acid ethyl ester (30.65 g, 0.103 mol) was suspended in isopropanol (300 ml). After addition of ammonium acetate (15.87 g, 0.206 mol), the mixture was heated to reflux for four hours. After evaporation of the volatiles, the residue was triturated with water (250 ml). Aqueous sodium hydroxide solution was added with stirring, to achieve pH=8 (from 4-5). After extraction with chloroform (3×300 ml), the combined organic layers were dried over sodium sulfate and evaporated to give 4′-chloro-4-hydroxy-[2,2′]bipyridinyl-6-carboxylic acid ethyl ester as a brownish solid.

C₁₃H₁₁ClN₂O₃, F_{w} 278.70.

¹H NMR (360 MHz, CDCl₃): δ=11.2-10.7 (broad s, 1H), 8.7-8.4 (broad m, 2H), 7.85 (s, 1H), 7.45 (s, 2H), 7.15-7.0 (broad m, 2H), 4.45 (qm, 2H), 1.40 (tm, 3H).

### Example 19

### 4′-chloro-6-hydroxymethyl-[2,2′]bipyridinyl-4-ol

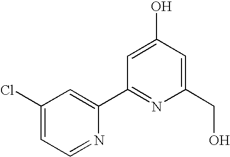

A mixture of 4′-chloro-4-hydroxy-[2,2′]bipyridinyl-6-carboxylic acid ethyl ester (11.15 g, 40 mmol) and sodium borohydride (9.08 g, 240 mmol) in dry dimethoxyethane (220 ml) was heated to reflux. Dry methanol (37 ml) was carefully added over a period of 3 hours. The brown suspension was cooled to room temperature. Aqueous HCl (4M) was added to achieve pH=5. Sodium hydroxide solution (4M) was added with stirring to pH 7-8, and the volatiles were removed in vacuo. The residue was chromatographed on silica gel with ethyl acetate/methanol/aq. ammonia 4:1:0.05 (v/v). 4′-chloro-6-hydroxymethyl-[2,2′]bipyridinyl-4-ol was thus obtained as a yellowish powder.

C₁₁H₉ClN₂O₂, F, 236.66.

¹H NMR (360 MHz, CD₃OD): δ=8.41 (d, J=5.4 Hz, 1H), 8.27 (d, J=1.8 Hz, 1H), 7.56 (dd, J=5.4, 1.8 Hz, 1H), 7.17 (s, 1H), 6.57 (s, 1H), 4.68 (s, 2H).

### Example 20

### 4′-Chloro-6-chloromethyl-[2,2′]bipyridinyl-4-ol

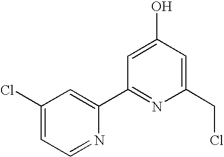

4′-Chloro-6-chloromethyl-[2,2′]bipyridinyl-4-ol (2.73 g, 11.5 mmol) was suspended in dry dichloromethane under an inert atmosphere at 0° C. Thionyl chloride (1.91 ml, 2.28 equiv.) was added dropwise. After 10 minutes, the cooling bath was removed, and the mixture was stirred for 30 minutes at room temperature. After cooling to 0° C., water (40 ml) was added, and the pH value of the mixture was adjusted to 7 with sodium carbonate solution. The organic layer was separated, and the aqueous layer was extracted with dichloromethane (2×150 ml). The combined organic extracts were dried over sodium sulfate, filtered and evaporated at 30° C. to give 4′-chloro-6-chloromethyl-[2,2′]bipyridinyl-4-ol as an off-white semisolid.

C₁₁H₈Cl₂N₂O, F_{w} 255.11.

¹H NMR (360 MHz, CD₃OD): δ=8.58 (dm, 1H); 8.32 (s, 1H); 7.50 (m, 2H); 6.88 (m, 1H); 4.69 (s, 2H).

### Example 20a

### 4′-dimethylamino-6-hydroxymethyl-[2,2′]bipyridinyl-4-ol

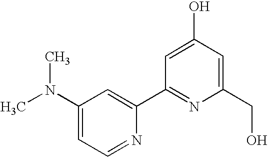

A solution of 4′-chloro-6-hydroxymethyl-[2,2′]bipyridinyl-4-ol (1.17 g, 4.96 mmol) in aqueous dimethylamine (60 ml, 7.9 M) was heated to 155° C. for 5 hours in a pressure reactor. After cooling to room temperature, the mixture was stirred for additional 14 hours at that temperature. The volatiles were removed in vacuo, and 4′-dimethylamino-6-hydroxymethyl-[2,2′]bipyridinyl-4-ol containing HCl was isolated as a beige solid.

C₁₃H₁₅N₃O₂, F_{w} 245.28.

¹H NMR (360 MHz, CD₃OD): δ=8.17 (d, J=6.8 Hz, 1H); 7.37 (d, J=2.7 Hz, 1H); 7.32 (s, 1H); 6.89 (dd, J=6.8, 2.7 Hz, 1H); 4.70 (s, 2H); 3.25 (s, 6H).

### Example 20b

### 6-chloromethyl-4′-dimethylamino-[2,2′]bipyridinyl-4-ol

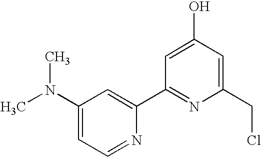

4′-Dimethylamino-6-hydroxymethyl-[2,2′]bipyridinyl-4-ol (2.6 g, 10.6 mmol) was suspended in dry dichloromethane under an inert atmosphere at 0° C. Thionyl chloride (1.75 ml, 24.2 mmol, 2.28 equiv.), was added dropwise with stirring. After 15 minutes, the cooling bath was removed, and the mixture was stirred for 2.5 hours at room temperature. After cooling to 0° C., water (100 ml) was added, and the pH-value was adjusted to 7 with sodium carbonate solution. The organic layer was separated, and the aqueous layer was extracted with chloroform (4×200 ml). The combined organic extracts were dried over sodium sulfate, filtered and evaporated to give 6-chloromethyl-4′-dimethylamino-[2,2′]bipyridinyl-4-ol as colorless powder. The crude product was used in the next step without further purification.

C₁₃H₁₄ClN₃O, F_{w} 263.73.

### Example 20c

### 4,4′-dichloro-[2,2′]bipyridinyl-6-carboxylic acid ethyl ester

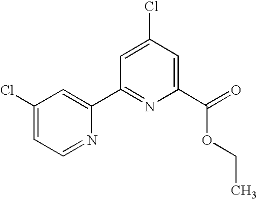

To a solution of phosphorus pentachloride (1.5 g, 7.2 mmol) in phosphoroxy trichloride (38 ml) was added 4′-chloro-4-hydroxy-[2,2′]bipyridinyl-6-carboxylic acid ethyl ester (836 mg, 3.0 mmol) in small portions at room temperature. The mixture was heated to reflux for 3 hours. After cooling to room temperature, the volatiles were evaporated and the residue was partitioned between dichloromethane and a saturated aqueous solution of sodium carbonate. The combined organic extracts were dried over sodium sulfate, filtered, and evaporated to yield 4,4′-dichloro-[2,2′]bipyridinyl-6-carboxylic acid ethyl ester as a brownish powder.

C₁₃H₁₀Cl₂N₂O₂, F_{w} 297.14.

¹H NMR (360 MHz, CDCl₃): δ=8.53 (d, J=1.8 Hz, 1H); 8.47-8.48 (m, 2H); 8.02 (d, J=1.8 Hz, 1H); 7.28 (dd, J=5.4, 1.8 Hz, 1H); 4.43 (q, J=7.2 Hz, 2H); 1.40 (t, J=6.8 Hz, 3H).

### Example 20d

### (4,4′-dichloro-[2,2′]bipyridinyl-6-yl)-methanol

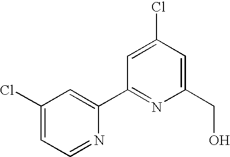

A mixture of 4,4′-dichloro-[2,2′]bipyridinyl-6-carboxylic acid ethyl ester (462 mg, 1.56 mmol) and sodium borohydride (353 mg, 9.34 mmol) in dry dimethoxyethane (15 ml) was heated to reflux. A solution of dry methanol (3 ml) in dimethoxyethane (15 ml) was carefully added over a period of 4 hours. After cooling to room temperature, an aqueous solution of hydrochloric acid (4 M) was added to achieve pH 5. The mixture was stirred for 4 days at room temperature. Sodium hydroxide solution (2 M) was added to adjust the pH to 8, and the volatiles were removed in vacuo. The residue was chromatographed on silica gel with hexane/ethyl acetate 1:1. (4,4′-Dichloro-[2,2′]bipyridinyl-6-yl)-methanol was thus obtained as brownish powder.

C₁₁H₈Cl₂N₂O, F_{w} 255.11.

¹H NMR (360 MHz, CDCl₃): δ=8.50 (d, J=6.8 Hz, 1H); 8.34 (d, J=2.7 Hz, 1H); 8.26 (s, 1H); 7.22-7.31 (m, 2H); 4.75 (s, 2H), 3.15 (s, br, 1H, OH).

### Example 20e

### (4,4′-bis-dimethylamino-[2,2′]bipyridinyl-6-yl)-methanol

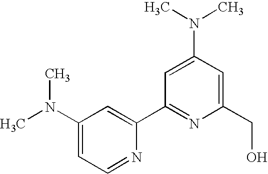

A solution of (4,4′-dichloro-[2,2′]bipyridinyl-6-yl)-methanol (235 mg, 0.921 mmol) in aqueous dimethylamine (25 ml, 7.9 M) was heated to 160° C. for 12 hours and 16 h at room temperature in a pressure reactor. After evaporation of the volatiles, 4,4′-bis-dimethylamino-[2,2′]bipyridinyl-6-yl)-methanol was obtained as a beige solid.

C₁₅H₂₀N₄O, F_{w} 272.35.

¹H NMR (360 MHz, CD₃OD): δ=8.02 (d, J=5.9 Hz, 1H); 7.36 (d, J=2.3 Hz, 1H); 7.19 (d, J=1.8 Hz, 1H); 6.71 (d, J=1.4 Hz, 1H); 6.66 (dd, J=6.8, 2.7 Hz, 1H); 4.57 (s, 2H); 3.07 (s, 6H); 3.03 (s, 6H).

### Example 20f

### 6-chloromethyl-N,N,N′,N′-tetramethyl-[2,2′]bipyridinyl-4,4′-diamine

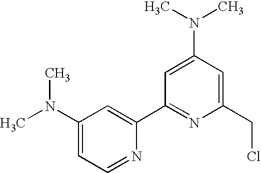

(4,4′-bis-dimethylamino-[2,2′]bipyridinyl-6-yl)-methanol (250 mg, 0.918 mmol) was suspended in dry dichloromethane under an inert atmosphere at 0° C. Thionyl chloride (0.152 ml, 2.1 mmol, 2.28 equiv.) was added dropwise with stirring. After 15 minutes, the cooling bath was removed, and the mixture was stirred for 2 hours at room temperature. After cooling to 0° C., water (10 ml) was added, and the pH-value of the mixture was adjusted to 8 with sodium carbonate solution. The organic layer was separated, and the aqueous layer was extracted with dichloromethane (3×50 ml), The combined organic extracts were dried over sodium sulfate, filtered and evaporated to give 6-chloromethyl-N,N,N′,N′-tetramethyl-[2,2′]bipyridinyl-4,4′-diamine as a brown oil. This compound was used in the next step without purification.

C₁₅H₁₉ClN₄, F_{w} 290.80.

### Synthesis of Ligands and Complexes

### Example 21

### 6-{[methyl-(2-pyridin-2-yl-ethyl)-amino]-methyl}-2-pyridin-2-yl-pyrimidin-4-ol

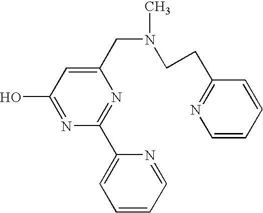

A mixture containing 6-chloromethyl-2-pyridin-2-yl-pyrimidin-4-ol (222 mg, 1 mmol), methyl-(2-pyridin-2-yl-ethyl)-amine (136 mg, 1 mmol), and triethylamine (202 mg, 2 mmol) in tetrahydrofuran (35 ml) was refluxed for three days. After filtration from a white precipitate, the mother liquor was concentrated in vacuo. The pure product was isolated as a brownish oil via column chromatography (RP 18, acetonitrile/water 4:1).

C₁₈H₁₉N₅O, F_{w} 321.39.

¹H NMR (360 MHz, CDCl₃): δ=10.94 (br s, 1H); 8.64 (d, J=4.5 Hz, 1H); 8.53 (d, J=4.5 Hz, 1H); 8.43 (d, J=7.7 Hz, 1H); 7.88 (dd, J=7.7, 1.4 Hz, 2H); 7.60 (dd, J=7.7, 1.4 Hz, 1H); 7.47 (dd, J=6.8, 5.0 Hz, 1H); 7.20 (d, J=7.7 Hz, 1H); 7.12 (dd, J=6.8, 5.0 Hz, 1H). 3.58 (s, 2H); 3.1-2.9 (m, 4H); 2.42 (s, 3H).

### Example 22

### 6-{[(4-methoxy-3,5-dimethyl-pyridin-2-ylmethyl)-methyl-amino]-methyl}-2-pyridin-2-yl-pyrimidin-4-ol

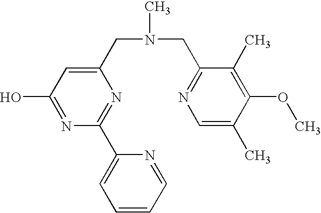

A mixture containing 6-chloromethyl-2-pyridin-2-yl-pyrimidin-4-ol (280 mg, 1.3 mmol), (4-methoxy-3,5-dimethyl-pyridin-2-ylmethyl)-methyl-amine (234 mg, 1.3 mmol), and triethylamine (360 μl, 4 mmol) in tetrahydrofuran (10 ml) was refluxed for three days. After evaporation of the volatiles, the pure product was isolated as a white solid via column chromatography (RP 18, methanol/water 7:5).

C₂₀H₂₃N₅O₂, F_{w} 365.44.

¹H NMR (360 MHz, DMSO-d6): δ=8.67 (d, J=4.5 Hz, 1H); 8.29 (d, J=7.5 Hz, 1H); 8.16 (s, 1H); 7.95 (tm, 1H); 7.55-7.49 (m, 1H); 6.13 (s, 1H); 3.69 (s, 3H); 3.67 (s, 2H); 3.42 (s, 2H); 2.27 (s, 3H); 2.20 (s, 3H); 2.16 (s, 3H).

### Example 23

### 6-[(methyl-pyridin-2-ylmethyl-amino)-methyl]-[2,2′]bipyridinyl-4-ol

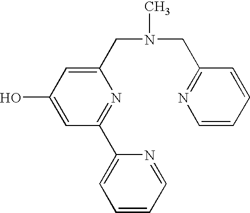

A mixture of 6-chloromethyl-[2,2′]bipyridinyl-4-ol (100 mg, 0.45 mmol), potassium carbonate (373 mg, 2.7 mmol), and methyl-pyridin-2-ylmethyl-amine (67 mg, 0.54 mmol) was refluxed for 16 hours. After filtration and evaporation of the volatiles, the product is isolated by chromatography (silica gel, chloroform/methanol) as a beige semisolid.

¹H NMR (360 MHz, CDCl₃): δ=7.89 (d, J=8.1 Hz, 1H); 7.80 (m, 1H); 7.63 (m, 1H); 7.45 (d, J=7.7 Hz, 1H); 7.38-7.32 (m, 1H); 7.17-7.12 (m, 1H); 7.07 (d, J=1.8 Hz, 1H); 6.41 (d, J=1.3 Hz, 1H); 3.85 (s, 2H); 3.62 (s, 2H); 2.41 (s, 3H).

### Example 24

### 4′-chloro-6-{[methyl-(2-pyridin-2-yl-ethyl)-amino]-methyl}-[2,2′]bipyridinyl-4-ol

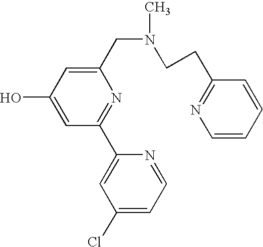

A mixture containing 4′-chloro-6-chloromethyl-[2,2′]bipyridinyl-4-ol (252 mg, 0.99 mmol), methyl-(2-pyridin-2-yl-ethyl)-amine (137 μl, 0.99 mmol), and triethylamine (550 μl, 1.99 mmol) in tetrahydrofuran (25 ml) was refluxed for three days. After evaporation of the volatiles, the residue was taken up in water and extracted with chloroform at pH=7.5 (3×100 ml). The organic phase was dried over sodium sulfate, filtered, and evaporated. The pure product was isolated as a brownish oil via column chromatography (RP 18, acetone/water 7:3).

C₁₉H₁₉ClN₄O, F_{w} 354.84.

¹H NMR (360 MHz, CDCl₃): δ=10.48 (br s, 1H); 8.34 (d, J=6.4 Hz, 1H); 8.28 (d, J=4.5 Hz, 1H); 7.65 (s, 1H); 7.40-7.33 (tm, 1H); 7.25-7.20 (dm, 1H); 6.98 (d, J=7.7 Hz, 1H); 6.88 (dd, J=7.2, 5.0 Hz, 1H); 6.73 (br s, 1H); 6.15 (br s, 1H); 3.41 (s, 2H); 2.91-2.72 (m, 4H); 2.21 (s, 3H).

### Example 25

### 6-{[methyl-(2-pyridin-2-yl-ethyl)-amino]-methyl}-4′-pyrrolidin-1-yl-[2,2′]bipyridinyl-4-ol

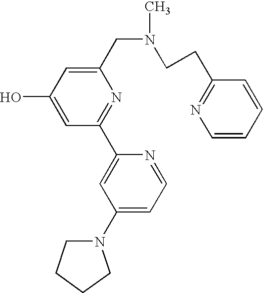

A mixture of 4′-chloro-6-{[methyl-(2-pyridin-2-yl-ethyl)-amino]-methyl}-[2,2′]bipyridinyl-4-ol (138 mg, 0.39 mmol), pyrrolidine (643 μl, 7.8 mmol), and chlorobenzene (10 ml) was heated for one day to 90° C. in the presence of a few grains of zinc(II) chloride. After evaporation of the volatiles, the residue was adjusted to 8 in water and extracted with chloroform (2×100 ml). The organic phase was dried over sodium sulfate, filtered, and evaporated. The pure product was obtained as a yellow oil after chromatography on aluminium oxide (chloroform).

C₂₃H₂₇N₅O, F_{w} 389.50.

¹H NMR (360 MHz, CDCl₃): δ=10.70 (br s, 1H); 8.46 (d, J=4.0 Hz, 1H); 8.19 (d, J=5.9 Hz, 1H); 7.52-7.48 (tm, 1H); 7.18 d, J=7.7 Hz, 1H); 7.20 (dd, J=7.2, 5.4 Hz, 1H); 6.93-6.87 (m, 2H); 6.42 (dd, J=5.4, 1.8 Hz, 1H); 6.26 (s, 1H); 3.55 (s, 2H); 3.40-3.30 (m, 4H); 3.08-2.88 (m, 4H); 3.35 (s, 3H); 2.08 (m, 4H).

### Example 26

### 4′-chloro-6-[(methyl-pyridin-2-ylmethyl-amino)-methyl]-[2,2′]bipyridinyl-4-ol

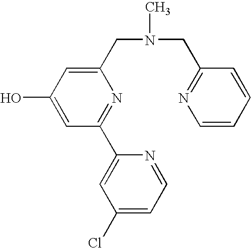

A mixture containing 4′-chloro-6-chloromethyl-[2,2′]bipyridinyl-4-ol (2.26 g, 8.9 mmol), methyl-pyridin-2-ylmethyl-amine (1.19 g, 9.75 mmol), and triethylamine (4.9 ml, 35 mmol) in tetrahydrofuran (200 ml) was refluxed for two days. After evaporation of the volatiles, the residue was taken up in water and extracted with dichloromethane at pH=8 (2×250 ml). The organic phase was dried over sodium sulfate, and evaporated. The pure product was isolated as a brownish oil via column chromatography (RP 18, methanol/water 4:1).

C₁₈H₁₇ClN₄O, F_{w} 340.82.

¹H NMR (360 MHz, CDCl₃): δ=11.47 (br s, 1H); 8.67-8.58 (m, 2H); 7.86 (s, 1H); 7.68 (ddd, J=7.7, 7.7, 1.8 Hz, 1H); 7.49-7.36 (m, 2H); 7.25-7.16 (m, 1H); 6.95 (br s, 1H); 6.36 (br s, 1H); 3.85 (s, 2H); 3.59 (s, 2H); 2.44 (s, 3H).

### Example 27

### 6-[(methyl-pyridin-2-ylmethyl-amino)-methyl]-4′-pyrrolidin-1-yl-[2,2′]bipyridinyl-4-ol

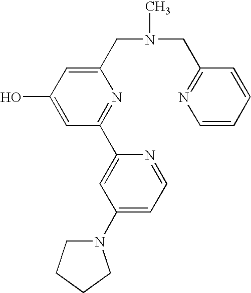

A mixture of 4′-chloro-6-[(methyl-pyridin-2-ylmethyl-amino)-methyl]-[2,2′]bipyridinyl-4-ol (574 mg, 1.68 mmol), pyrrolidine (2.79 ml, 33.7 mmol), and chlorobenzene (45 ml) was heated for two days under reflux in the presence of a few grains of zinc(II) chloride. After evaporation of the volatiles, the residue was neutralized in water by addition of dilute hydrochloric acid, and extracted with chloroform (2×100 ml). The organic phase was dried over sodium sulfate, filtered, and evaporated. The pure product was obtained as a yellow oil after chromatography on aluminium oxide (chloroform).

C₂₂H₂₅N₅O, F_{w} 375.48.

¹H NMR (360 MHz, CDCl₃): δ=11.4 (br s, 1H); 8.61 (d, J=4.5 Hz, 1H); 8.29 (d, J=5.9 Hz, 1H); 7.68 (ddd, J=7.7, 7.7, 1.8 Hz, 1H); 7.56 (d, J=7.7 Hz, 1H); 7.22-7.15 (m, 1H); 6.91 (dd, J=4.5, 2.2 Hz, 1H); 6.45 (dd, J=5.9, 2.2 Hz, 1H); 6.29 (d, J=1.8 Hz, 1H); 3.84 (s, 2H); 3.56 (s, 2H); 3.40-3.35 (m, 4H); 2.41 (s, 3H); 2.10-2.05 (m, 4H).

### Example 28

### 4′-[(2-hydroxy-ethyl)-methyl-amino]-6-[(methyl-pyridin-2-ylmethyl-amino)-methyl]-[2,2′]bipyridinyl-4-ol

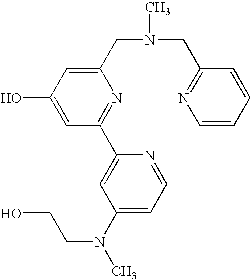

A mixture of 4′-chloro-6-[(methyl-pyridin-2-ylmethyl-amino)-methyl]-[2,2′]bipyridinyl-4-ol (485 mg, 1.42 mmol), N-methylamino ethanol (2.13 g, 28.4 mmol), and chlorobenzene (40 ml) was heated for two days under reflux in the presence of a few grains of zinc(II) chloride. After evaporation of the volatiles, the residue was neutralized in water by addition of dilute hydrochloric acid, and extracted with chloroform (3×200 ml). The organic phase was dried over sodium sulfate, filtered, and evaporated. The pure product was obtained as a beige solid after chromatography on aluminium oxide (chloroform/methanol 95:5).

C₂₁H₂₅N₅O₂, F_{w} 379.47.

¹H NMR (360 MHz, CDCl₃): δ=11.5 (br s, 1H); 8.58-8.53 (m, 1H); 8.21 (d, J=5.8 Hz, 1H); 7.68 (ddd, J=7.7, 7.7, 1.3 Hz, 1H); 7.54 (d, J=7.7 Hz, 1H); 7.39 (d, J=2.3 Hz, 1H); 7.26 (d, J=2.3 Hz, 1H); 7.22-7.15 (m, 1H); 6.60-6.55 (ddm, 1H); 6.23 (d, J=1.8 Hz, 1H); 5.70 (br s, 1H); 3.87 (t, J=5.9 Hz, 2H); 3.77 (s, 2H); 3.65 (t, J=5.9 Hz, 2H); 3.55 (s, 2H); 3.09 (s, 3H); 2.36 (s, 3H).

### Example 29

### 4′-(4-methyl-piperazin-1-yl)-6-[(methyl-pyridin-2-ylmethyl-amino)-methyl]-[2,2′]bipyridinyl-4-ol

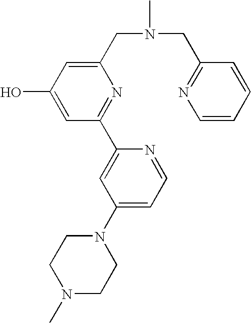

A mixture of 4′-chloro-6-[(methyl-pyridin-2-ylmethyl-amino)-methyl]-[2,2′]bipyridinyl-4-ol (1.31 g, 3.84 mmol), N-methyl piperazine (8.54 ml, 77 mmol), and chlorobenzene (40 ml) was heated for one day under reflux in the presence of zinc(II) chloride (25 mg). After evaporation of the volatiles, the residue was neutralized in water by addition of dilute hydrochloric acid. The pure product was obtained as a beige oil after chromatography on aluminium oxide (chloroform).

C₂₃H₂₈N₆O, F_{w} 404.52.

¹H NMR (360 MHz, D₂O): δ=8.01 (d, J=4.0 Hz, 1H); 7.73 (d, J=6.3 Hz, 1H); 7.35 (ddd, J=7.7, 7.7, 1.8 Hz, 1H); 7.05 (d, J=7.7 Hz, 1H); 6.96-6.88 (m, 1H); 6.73 (dd, J=2.3 Hz, 1H); 6.45-6.37 (m, 2H); 6.07-6.02 (d, J=2.3 Hz, 1H); 3.28 (s, 2H); 3.25 (s, 2H); 3.03 (br m, 4H); 2.21 (br m, 4H); 1.99 (s, 3H); 1.97 (s, 3H).

### Example 30

### 4-{4′-hydroxy-6′-[(methyl-pyridin-2-ylmethyl-amino)-methyl]-[2,2′]bipyridinyl-4-yl}-1,1-dimethyl-piperazin-1-ium iodide

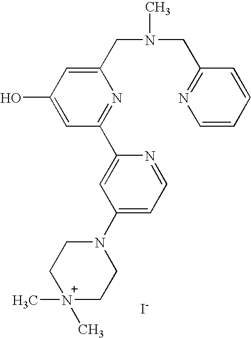

A stirred solution of 4′-(4-methyl-piperazin-1-yl)-6-[(methyl-pyridin-2-ylmethyl-amino)-methyl]-[2,2′]bipyridinyl-4-ol (283 mg, 0.7 mmol) in acetonitrile (1 ml), at 0° C., was treated with 1 equiv. methyl iodide stock solution in acetonitrile. The resulting suspension was diluted with acetonitrile (1 ml), and stirred overnight at room temperature. The product was isolated as a white solid after filtration and washing with acetonitrile (2 ml).

C₂₄H₃₁1N₆O, F_{w} 419.55.

¹H NMR (360 MHz, ₃): δ=8.30-8.16 (m, 2H); 7.56 (m, 1H); 7.30 (dm, 1H); 7.20-7.10 (m, 2H); 6.85 (dm, 1H); 6.72 (m, 1H); 6.30 (dm, 1H); 3.75 (tm, 4H); 3.65-3.50 (m, 8H); 3.20 (s, 6H); 2.30 (s, 3H).

### Example 31

### 4′-chloro-6-{[(4-dimethylamino-pyridin-2-ylmethyl)-methyl-amino]-methyl}-[2,2′]bipyridinyl-4-ol

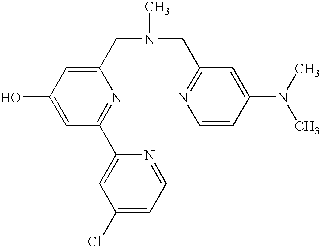

A mixture containing 4′-chloro-6-chloromethyl-[2,2′]bipyridinyl-4-ol (280 mg, 1.1 mmol), dimethyl-(2-methylaminomethyl-pyridin-4-yl)-amine (209 mg, 1.26 mmol), and triethylamine (610 μl, 4.4 mmol) in tetrahydrofuran (25 ml) was refluxed for 2 days. After evaporation, the pure product was isolated as a beige solid via column chromatography (RP 18, methanol/water 9:1).

C₂₀H₂₂ClN₅O, F_{w} 383.88.

¹H NMR (360 MHz, ₃): δ=8.58 (d, J=5.0 Hz, 1H); 8.17 (s, 1H); 7.90 (m, 1H); 7.50 (dm, 1H); 7.03 (s, 1H); 6.70 (m, 1H); 6.55-6.40 (m, 2H); 3.58 (s, 2H); 3.40 (s, 2H); 2.95 (s, 6H); 2.29 (s, 3H).

### Example 32

### 6-{[(4-dimethylamino-pyridin-2-ylmethyl)-methyl-amino]-methyl}-4′-pyrrolidin-1-yl-[2,2′]bipyridinyl-4-ol

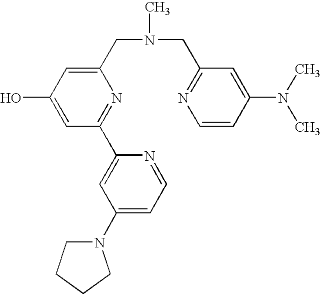

A mixture of 4′-chloro-6-{[(4-dimethylamino-pyridin-2-ylmethyl)-methyl-amino]-methyl}-[2,2′]bipyridinyl-4-ol (113 mg, 294 μmol), pyrrolidine (487 μl, 5.9 mmol), and chlorobenzene (10 ml) was heated for 18 hours under reflux in the presence of a few grains of zinc(II) chloride. After evaporation of the volatiles, the residue was neutralized in water by addition of dilute hydrochloric acid. The pure product was obtained after chromatography on aluminium oxide (chloroform/methanol 20:1).

C₂₄H₃₀N₆O, F_{w} 418.55.

¹H NMR (360 MHz, CDCl₃): δ=8.16-8.11 (ddm, 2H); 6.86 (dm, 2H); 6.66 (d, J=2.7 Hz, 1H); 6.37-6.33 (dm, 2H); 6.23 (d, J=1.8 Hz, 1H); 2.22 (s, 2H); 2.24 (s, 2H); 3.32-3.27 (m, 4H); 2.93 (2 s, 6H); 3.34 (s, 3H); 2.25-1.98 (m, 4H).

### Example 33

### 6-{[(4-dimethylamino-pyridin-2-ylmethyl)-methyl-amino]-methyl}-4′-[(2-hydroxy-ethyl)-methyl-amino]-[2,2′]bipyridinyl-4-ol

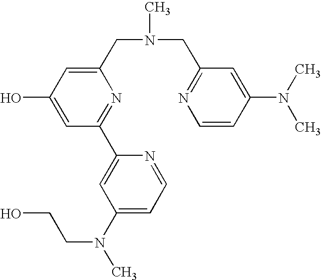

A mixture of 4′-chloro-6-{[(4-dimethylamino-pyridin-2-ylmethyl)-methyl-amino]-methyl}-[2,2′]bipyridinyl-4-ol (128 mg, 333 μmol), N-methylamino ethanol (533 μl, 6.66 mmol), and chlorobenzene (10 ml) was heated for 18 hours under reflux in the presence of a few grains of zinc(II) chloride. After evaporation of the volatiles, the residue was neutralized in water by addition of dilute hydrochloric acid. The pure product was obtained as a yellow oil after chromatography on aluminium oxide (chloroform/methanol 10:1).

C₂₃H₃₀N₆O₂, F_{w} 422.53.

¹H NMR (360 MHz, CDCl₃): δ=8.07 (2 d, J=5.8 Hz, 2H); 7.26 (d, J=2.3 Hz, 1H); 7.14 (d, J=2.3 Hz, 1H); 6.63 (d, J=2.3 Hz, 1H); 6.49 (dd, J=5.8, 2.3 Hz, 1H); 6.35-6.28 (ddm, 1H); 6.16 (d, J=1.8 Hz); 3.81-3.75 (tm, 2H); 3.61 (s, 2H); 3.59-3.52 (tm, 2H); 3.43 (s, 2H); 3.00 (s, 3H); 2.89 (s, 6H); 2.99 (s, 3H).

### Example 34

### 4′-chloro-6-{[(4-methoxy-3,5-dimethyl-pyridin-2-ylmethyl)-methyl-amino]-methyl}-[2,2′]bipyridinyl-4-ol

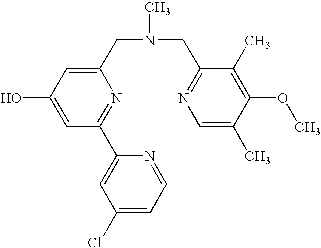

A mixture containing 6-chloromethyl-2-pyridin-2-yl-pyrimidin-4-ol (983 mg, 3.85 mmol), (4-methoxy-3,5-dimethyl-pyridin-2-ylmethyl)-methyl-amine hydrochloride (764 mg, 4.24 mmol), and triethylamine (2.15 ml, 15.4 mmol) in tetrahydrofuran (100 ml) was refluxed for two days. The volatiles were removed in vacuo, and water (50 ml) was added. After extraction with dichloromethane (2×200 ml), the organic layers were collected, dried over sodium sulfate, and evaporated. The pure product was isolated as a yellowish solid via column chromatography (RP 18, methanol/water 4:1).

C₂₁, H₂₃ClN₄O₂, F_{w} 398.90.

¹H NMR (360 MHz, CDCl₃): δ=8.60 (dm, 1H); 8.30 (s, 1H); 7.81 (s, 1H); 7.40 (dm, 1H); 6.87 (br s, 1H); 6.29 (br s, 1H); 3.80 (s, 2H); 3.76 (s, 3H); 3.50 (s, 2H); 2.45 (s, 3H); 2.32 (s, 3H); 2.25 (s, 3H).

### Example 35

### 4′-[(2-hydroxy-ethyl)-methyl-amino]-6-{[(4-methoxy-3,5-dimethyl-pyridin-2-ylmethyl)-methyl-amino]-methyl}-[2,2′]bipyridinyl-4-ol

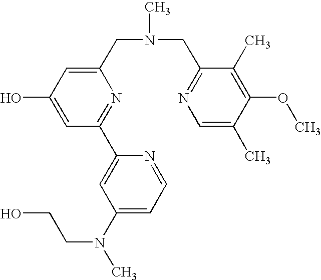

A mixture of 4′-chloro-6-{[(4-methoxy-3,5-dimethyl-pyridin-2-ylmethyl)-methyl-amino]-methyl}-[2,2′]bipyridinyl-4-ol (250 mg, 0.627 mmol), N-methylamino ethanol (1.0 ml, 12.5 mmol), and chlorobenzene (15 ml) was heated for two days under reflux in the presence of zinc(II) chloride (4 mg). After evaporation of the volatiles, the residue was neutralized in water by addition of dilute hydrochloric acid, and extracted with chloroform (2×200 ml). The organic phase was dried over sodium sulfate, filtered, and evaporated. The pure product was obtained as a brownish semisolid after chromatography on aluminium oxide (chloroform/methanol 9:1).

C₂₄H₃₁N₅O₃, F_{w} 437.55.

¹H NMR (360 MHz, CDCl₃): δ=8.22 (dm, 1H); 8.06 (s, 1H); 7.40-7.21 (m, 2H); 6.60 (dm, 1H); 6.20 (s, 1H); 3.80-3.52 (m, 4H); 3.68 (2 s, 5H); 3.40 (s, 2H); 3.05 (s, 3H); 2.52 (s, 3H); 2.18 (s, 3H); 2.08 (s, 3H).

### Example 36

### 6-{[(4-methoxy-3,5-dimethyl-pyridin-2-ylmethyl)-methyl-amino]-methyl}-4′-pyrrolidin-1-yl-[2,2′]bipyridinyl-4-ol

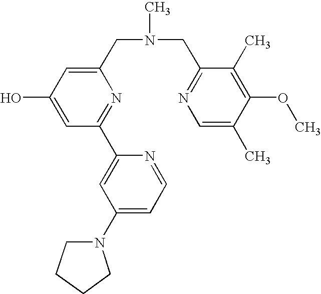

A mixture of 4′-chloro-6-{[(4-methoxy-3,5-dimethyl-pyridin-2-ylmethyl)-methyl-amino]-methyl}-[2,2′]bipyridinyl-4-ol (250 mg, 0.627 mmol), N-methylamino ethanol (1.0 ml, 12.5 mmol), and chlorobenzene (15 ml) was heated for one day under reflux in the presence of zinc(II) chloride (4 mg). After evaporation of the volatiles, the residue was neutralized in water by addition of dilute hydrochloric acid, and extracted with chloroform (2×150 ml). The organic phase was dried over sodium sulfate, filtered, and evaporated. The pure product was obtained as a beige semisolid after chromatography on aluminium oxide (chloroform).

C₂₅H₃₁N₅O₂, F_{w} 433.56.

¹H NMR (360 MHz, CDCl₃): δ=8.31 (s, 1H); 8.25 (d, J=5.4 Hz, 1H); 6.90 (d, J=2.2 Hz, 1H); 6.89 (d, J=2.2 Hz, 1H); 6.46-6.41 (ddm, 1H); 6.26 (d, J=2.2 Hz, 1H); 3.80 (s, 2H); 3.74 (s, 3H); 3.49 (s, 2H); 3.36 (tm, 4H); 2.40 (s, 3H); 2.38 (s, 3H); 3.27 (s, 3H); 2.09 (tm, 4H).

### Example 37

### 6-{[(4-methoxy-3,5-dimethyl-pyridin-2-ylmethyl)-methyl-amino]-methyl}-4′-(4-methyl-piperazin-1-yl)-[2,2]bipyridinyl-4-ol

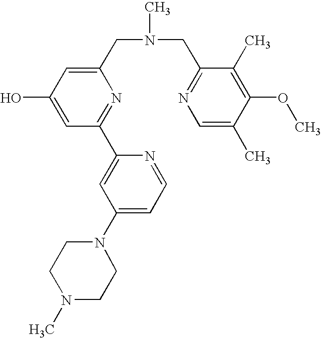

A mixture of 4′-chloro-6-{[(4-methoxy-3,5-dimethyl-pyridin-2-ylmethyl)-methyl-amino]-methyl}-[2,2′]bipyridinyl-4-ol (294 mg, 0.737 mmol), N-methyl piperazine (1.64 ml, 14.7 mmol), and chlorobenzene (18 ml) was heated for one day under reflux in the presence of zinc(II) chloride (5 mg). After evaporation of the volatiles, the residue was neutralized in water by addition of dilute hydrochloric acid, and extracted with chloroform (3×100 ml). The organic phase was dried over sodium sulfate, and filtered, and evaporated. The pure product was obtained as a beige semisolid after chromatography on aluminium oxide (chloroform).

C₂₆H₃₄N₆O₂, F_{w} 462.60.

¹H NMR (360 MHz, CDCl₃): δ=11.52 (br s, 1H); 8.26 (d, J=5.8 Hz, 1H); 8.23 (s, 1H); 7.12 (d, J=2.3 Hz, 1H); 6.79 (d, J=1.8 Hz, 1H); 6.65 (dd, J=5.8, 2.2 Hz, 1H); 6.19 (d, J=1.8 Hz, 1H); 3.72 (s, 2H); 3.67 (s, 3H); 3.42 (s, 2H); 3.34 (tm, 4H); 2.49 (tm, 4H); 2.34 (s, 3H); 2.29 (2 s, 6H); 2.18 (s, 3H).

### Example 38

### 4′-Chloro-6-{[(4-chloro-pyridin-2-ylmethyl)-methyl-amino]-methyl}-[2,2′]bipyridinyl-4-ol

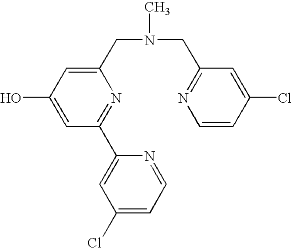

A mixture containing 6-chloromethyl-2-pyridin-2-yl-pyrimidin-4-ol (4.0 g, 15.7 mmol), (4-chloro-pyridin-2-ylmethyl)-methyl-amine (2.70 g, 17.3 mmol), and triethylamine (8.73 ml, 63 mmol) in tetrahydrofuran (200 ml) was refluxed for 1.5 days. After evaporation, the pure product was isolated as a brownish solid via column chromatography (RP 18, methanol/water 4:1).

C₁₈H₁₆Cl₂N₄O, F_{w} 375.26.

¹H NMR (360 MHz, ₃): δ=8.52 (d, J=5.0 Hz, 1H); 8.32 (dm, 1H); 7.79 (s, 1H); 7.46 (dm, 1H); 7.27 (dd, J=5.0, 1.8 Hz, 1H); 7.07 (qm, 1H); 6.87 (s, 1H); 6.26 (s, 1H); 3.67 (s, 2H); 3.51 (s, 2H); 2.27 (s, 3H).

### Example 39

### 6-{[methyl-(4-pyrrolidin-1-yl-pyridin-2-ylmethyl)-amino]-methyl}-4′-pyrrolidin-1-yl-[2,2′]bipyridinyl-4-ol

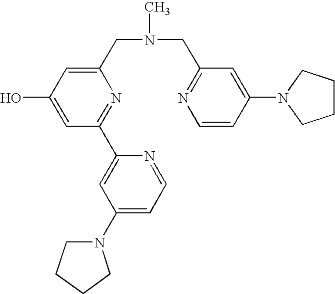

A mixture of 4′-chloro-6-{[(4-chloro-pyridin-2-ylmethyl)-methyl-amino]-methyl}-[2,2′]bipyridinyl-4-ol (750 mg, 2 mmol), pyrrolidine (5.0 ml, 60 mmol), and chlorobenzene (50 ml) was heated for one day under reflux in the presence of zinc(II) chloride (14 mg). After evaporation of the volatiles, the residue was neutralized in water by addition of dilute hydrochloric acid, and extracted with chloroform (3×100 ml). The organic phase was dried over sodium sulfate, filtered, and evaporated. The pure product was obtained as a beige semisolid after chromatography on aluminium oxide (dichloromethane/methanol 15:1).

C₂₆H₃₂N₆O, F_{w} 444.58.

¹H NMR (360 MHz, CDCl₃): δ=8.10 (2 m, 2H); 8.83 (2 s, 2H); 6.55 (d, J=2.3 Hz, 1H); 8.37 (dd, J=5.9, 2.3 Hz, 1H); 6.22 (2 m, 2H); 3.67 (s, 2H); 3.46 (s, 2H); 3.35-3.32 (2 tm, 8H); 2.35 (s, 3H), 2.05-1.95 (2 tm, 8H).

### Example 40

### 4′-[(2-hydroxy-ethyl)-methyl-amino]-6-[({4-[(2-hydroxy-ethyl)-methyl-amino]-pyridin-2-ylmethyl)}-methyl-amino)-methyl]-[2,2′]bipyridinyl-4-ol

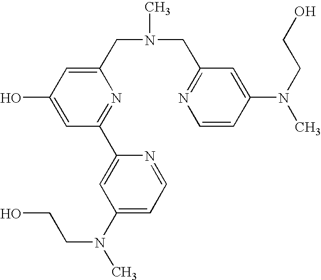

A mixture of 4′-chloro-6-{[(4-chloro-pyridin-2-ylmethyl)-methyl-amino]-methyl}-[2,2′]bipyridinyl-4-ol (1.12 g, 3 mmol), N-methylamino ethanol (0.96 ml, 12 mmol), and water (5 ml) was heated for one day under reflux in a pressure reactor. After evaporation of the volatiles, the residue was obtained as a grey semisolid via chromatography on aluminium oxide (chloroform/methanol 4:1).

C₂₄H₃₂N₆O₃, F_{w} 452.56.

¹H NMR (360 MHz, D₂O): δ=8.08 (d, J=7.2 Hz, 1H); 7.70 (d, J=7.7 Hz, 1H); 7.20 (m, 1H); 7.04 (m, 1H); 6.93 (m, 1H); 6.73 (m, 1H); 6.66 (m, 1H); 6.62 (dm, 1H); 3.90-3.44 (m, 12H); 3.19 (s, 3H); 2.98 (s, 3H); 2.52 (s, 3H).

### Example 40a

### 4′-dimethylamino-6-{[(4-dimethylamino-pyridin-2-ylmethyl)-methyl-amino]-methyl}-[2,2′]bipyridinyl-4-ol

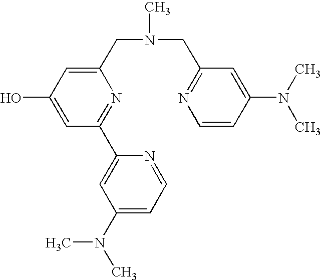

A mixture containing 6-chloromethyl-4′-dimethylamino-[2,2′]bipyridinyl-4-ol (1.06 g, 4.01 mmol) and dimethyl-(2-methylaminomethyl-pyridin-4-yl)-amine (0.80 g, 4.82 mmol), and triethylamine (3.35 ml, 24.1 mmol) in tetrahydrofuran (110 ml) was refluxed for 2 days. After filtration, the filtrate was evaporation, and the pure product was isolated as yellowish powder after column chromatography on aluminium oxide (chloroform/methanol 9:1).

C₂₂H₂₈N₆O, F_{w} 392.51.

¹H NMR (360 MHz, CDCl₃): δ=8.15 (d, J=5.9 Hz, 1H); 8.11 (d, J=5.9 Hz, 1H); 6.96 (d, J=2.3 Hz, 1H); 6.82 (d, J=2.3 Hz, 1H); 6.64 (d, J=2.3 Hz, 1H); 6.46 (dd, J=5.9, 2.3 Hz, 1H); 6.34 (dd, J=6.3, 2.7 Hz, 1H); 6.21 (d, J=1.8 Hz, 1H); 3.68 (s, 2H); 3.50 (s, 2H); 2.99 (s, 6H); 2.93 (s, 6H); 2.34 (s, 3H).

### Example 40b

### 6-{[(4-dimethylamino-pyridin-2-ylmethyl)-methyl-amino]-methyl}-N,N,N N′-tetramethyl-[2,2′]bipyridinyl-4,4′-diamine

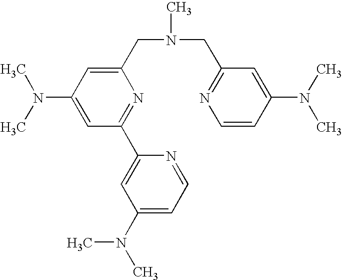

A mixture containing 6-chloromethyl-N,N,N′,N′-tetramethyl-[2,2′]bipyridinyl-4,4′-diamine (170 mg, 0.585 mmol) and dimethyl-(2-methylaminomethyl-pyridin-4-yl)-amine (116 mg, 0.702 mmol), and triethylamine (0.49 ml, 3.51 mmol) in tetrahydrofuran (15 ml) was refluxed for 20 hours. After evaporation, the pure product was isolated as brownish oil after column chromatography on aluminium oxide (chloroform/methanol 9:1).

C₂₄H₃₃N₇, F_{w} 419.58.

¹H NMR (360 MHz, CDCl₃): δ=8.18 (d, J=5.9 Hz, 1H); 8.06 (d, J=6.3 Hz, 1H); 7.62 (d, J=2.7 Hz, 1H); 7.46 (d, J=2.7 Hz, 1H); 6.77 (d, J=2.3 Hz, 1H); 6.71 (d, J=2.3 Hz, 1H); 6.40 (dd, J=5.9, 2.7 Hz, 1H); 6.29 (dd, J=5.9, 2.3 Hz, 1H); 3.71 (s, 2H); 3.67 (s, 2H); 3.01 (s, 6H); 2.99 (s, 6H); 2.89 (s, 6H); 2.35 (s, 3H).

### Example 40c

### 4′-dimethylamino-6-{[methyl-(4-pyrrol-1-yl-pyridin-2-ylmethyl)-amino]-methyl}-[2,2′]bipyridinyl-4-ol

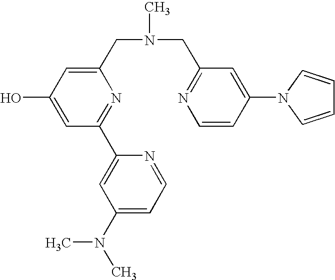

A mixture containing 6-chloromethyl-4′-dimethylamino-[2,2′]bipyridinyl-4-ol (117 mg, 0.446 mmol) and methyl-(4-pyrrol-1-yl-pyridin-2-ylmethyl)-amine (83 mg, 0.446 mmol), and triethylamine (0.37 ml, 2.68 mmol) in tetrahydrofuran (14 ml) was refluxed for 2.5 days. After filtration, the filtrate was evaporation, and the pure product was isolated as brownish powder after column chromatography on aluminium oxide (chloroform/methanol 9:1).

C₂₄H₂₆N₆O, F_{w} 414.51.

¹H NMR (360 MHz, CDCl₃): δ=8.43 (d, J=5.5 Hz, 1H); 8.10 (d, J=5.9 Hz, 1H); 7.54 (d, J=2.3 Hz, 1H); 7.14-7.16 (m, 2H); 7.09 (dd, J=5.4, 2.3 Hz, 1H); 6.93 (d, J=2.3 Hz, 1H); 6.82 (d, J=1.8 Hz, 1H); 6.45 (dd, J=6.3, 2.7 Hz, 1H); 6.26-6.27 (m, 2H); 6.21 (d, J=1.8 Hz, 1H); 3.74 (s, 2H); 3.53 (s, 2H); 2.98 (s, 6H); 2.34 (s, 3H).

### Example 41

### manganese complex with 4′-[(2-hydroxy-ethyl)-methyl-amino]-6-[({4-[(2-hydroxy-ethyl)-methyl-amino]-pyridin-2-ylmethyl}-methyl-amino)-methyl]-[2,2′]bipyridinyl-4-ol

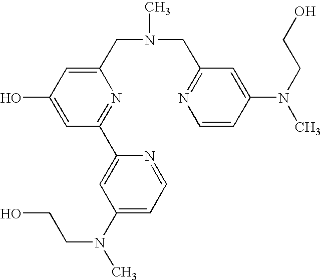

A solution of 4′-[(2-Hydroxy-ethyl)-methyl-amino]-6-[({4-[(2-hydroxy-ethyl)-methyl-amino]-pyridin-2-ylmethyl}-methyl-amino)-methyl]-[2,2′]bipyridinyl-4-ol (36 mg, 0.079 mmol) and manganese(II) chloride tetrahydrate (15 mg, 1 equiv.) is lyophilized to yield the complex C₂₄H₃₂Cl₂MnN₆O₃ (F, 578.41) as a yellow foam.

IR (cm⁻¹): 3351 (br, s); 2957 (s); 1638 (s); 1609 (s); 1582 (s); 1553 (s); 1516 (m); 1012 (s).

### Example 42

### manganese complex with 4′-[(2-hydroxy-ethyl)-methyl-amino]-6-[(methyl-pyridin-2-ylmethyl-amino)-methyl]-[2,2′]bipyridinyl-4-ol

with

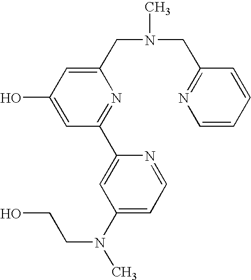

A solution of 4′-[(2-hydroxy-ethyl)-methyl-amino]-6-[(methyl-pyridin-2-ylmethyl-amino)-methyl]-[2,2′]bipyridinyl-4-ol (43 mg, 0.112 mmol) and manganese(II) chloride tetrahydrate (22 mg, 1 equiv.) is lyophilized to yield the complex C₂₁H₂₅Cl₂MnN₅O₂ (F_{w} 509.34) as a yellow foam.

IR (cm⁻¹): 3352 (br, s); 2926 (w); 1607 (s); 1581 (s); 1537 (w); 1438 (m); 1011 (s).

### Example 43

### Iron complex with 4′-[(2-hydroxy-ethyl)-methyl-amino]-6-[(methyl-pyridin-2-ylmethyl-amino)-methyl]-[2,2′]bipyridinyl-4-ol

with

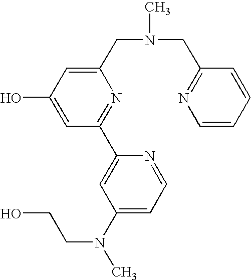

An aqueous 1.6 mM solution of 4′-[(2-Hydroxy-ethyl)-methyl-amino]-6-[(methyl-pyridin-2-ylmethyl-amino)-methyl]-[2,2′]bipyridinyl-4-ol and iron(II) chloride (1 equiv.) was prepared by mixing the components in an Erlenmeyer flask, and subsequently diluted to a final concentration of 50 μM.

UV-vis (extinction): 218 nm (min., 0.880); 247 nm (max., 1.566); 334 (shoulder, 0.350); 413 nm (shoulder, 0.124).

### APPLICATION EXAMPLES

### Application Example 1

### Stain bleaching in laundry at ambient temperature

1 g of a circular stain (BC01 Tea, CFT; WFK10.0 Carrot juice, WFK; CS20/2 Tomato, CFT; BC04 Curry, CFT) was added into a vial with 3 ml washing liquor. The liquor contained a standard washing agent (IEC 60456*) in a concentration of 7.5 g/l. The hydrogen peroxide concentration was 10 mmol/l. The catalyst concentration (1:1 in-situ complex of the ligand with manganese(II) chloride tetrahydrate or iron (ii) chloride in methanolic solution) was 25 μmol/l. The vial was shaken with a shaker for 50 minutes at ambient temperature. After the treatment the fabric was carefully rinsed and ironed. The brightness values Y according to the CIE standard procedure of the stained test fabrics were measured with a Gretag SPM 100 instrument prior to and after the treatment, respectively. The bleaching effect is given as ΔΔY, i.e. the difference between the brightness of the fabrics washed in presence and in the absence of a catalyst, respectively.

| Complex | | | | |
|---|---|---|---|---|
| with ligand | ΔΔY | | | ΔY |
| example | (Tea) | ΔΔY (Carrot Juice) | ΔΔY (Tomato) | Δ(Curry) |
| Ligand 23-Mn | 2.9 | 1.3 | 0.6 | 7.4 |
| Ligand 27-Mn | 5.3 | 1.3 | 4.7 | 8.2 |
| Ligand 28-Mn | 6.0 | 2.0 | 5.0 | 8.5 |
| Ligand 28-Fe | 4.7 | 3.8 | 5.1 | 5.3 |
| Ligand 29-Mn | 6.4 | 3.1 | 4.4 | 8.3 |
| Ligand 30-Mn | 7.0 | 3.6 | 7.4 | 7.1 |
| Ligand 32-Mn | 6.4 | 3.1 | 5.6 | 8.5 |
| Ligand 33-Mn | 5.5 | 3.9 | 6.0 | 9.1 |
| Ligand 36-Mn | 4.0 | 3.3 | 4.9 | 6.3 |
| Ligand 39-Mn | 5.1 | 4.2 | 8.8 | 7.9 |
| Ligand 40-Mn | 5.8 | 2.7 | 5.5 | 10.3 |
| Ligand 40a-Mn | 7.0 | 5.0 | 5.0 | 3.0 |
| Ligand 40b-Mn | 1.7 | 2.7 | 2.3 | 3.7 |
| Ligand 40c-Mn | 3.9 | 2.5 | 1.3 | 4.9 |

It can be seen from the above table, the presence of complexes of the present invention considerably improves the bleach performance of hydrogen peroxide (ΔΔY=0) on various bleachable stains.

### Application Example 2

### (Stain bleaching in laundry at 40° C.)

7.5 g of white cotton fabric and 2.5 g of tea-stained cotton fabric were treated in 80 ml of washing liquor. The liquor contained a standard detergent (IEC 60456 A*) in a concentration of 7.5 g/l. The hydrogen peroxide concentration was 10 mmol/l. The catalyst concentration (1:1 in-situ complex of the ligand with manganese(II) chloride tetrahydrate in methanolic solution) was 20 μmol/l. The washing process was carried out in a steel beaker in a LINITEST apparatus for 30 minutes at 40° C. To evaluate the bleaching results, the increase in brightness ΔΔY of the stains relative to reference experiments without the addition of catalyst (brightness according to CIE) was determined spectrophotometrically.

| Complex with ligand example | ΔΔY |
|---|---|
| Ligand 23/Mn | 2.1 |
| Ligand 27/Mn | 2.7 |
| Ligand 28/Mn | 1.5 |
| Ligand 29/Fe | 5.0 |
| Ligand 32/Mn | 4.5 |
| Ligand 33/Mn | 3.9 |
| Ligand 30/Mn | 4.5 |
| Ligand 36/Mn | 1.6 |
| Ligand 39/Mn | 4.0 |
| Ligand 40/Mn | 4.9 |
| Ligand 40a/Mn | 3.0 |
| Ligand 40b/Mn | 1.0 |
| Ligand 40c/Mn | 2.2 |

A significant increase in brightness compared with the catalyst-free washing process (ΔΔY=0) is observed as indicated in the above table.

### Application Example 3

### (Stain bleaching in laundry with peracetic acid as oxidant)

1 g of a circular stain (BC01 Tea, CFT; WFK 0.0 Carrot juice, WFK; CS20/2 Tomato, CFT; BC04 Curry, CFT) was added into a vial with 3 ml washing liquor. The liquor contained a standard washing agent (IEC 60456*) in a concentration of 7.5 g/l. The peracetic acid concentration was 3 mmol/l. The catalyst concentration (1:1 in-situ complex of the ligand with manganese(II) chloride tetrahydrate or iron (ii) chloride in methanolic solution) was 10 μmol/l. The vial was shaken with a shaker for 50 minutes at ambient temperature. After the treatment the fabric was carefully rinsed and ironed. The brightness values Y according to the CIE standard procedure of the stained test fabrics were measured with a Gretag SPM 100 instrument prior to and after the treatment, respectively. The bleaching effect is given as ΔΔY, i.e. the difference between the brightness of the fabrics washed in presence vs. the absence of a catalyst, respectively.

| Complex | | | | |
|---|---|---|---|---|
| with ligand | ΔΔY | ΔΔY | | |
| example | (Tea) | (Carrot Juice) | ΔΔY (Tomato) | ΔΔY (Curry) |
| Ligand 32-Mn | 5.6 | 2.5 | 1.2 | 5.3 |
| Ligand 33-Mn | 6.1 | 3.4 | 1.5 | 5.7 |
| Ligand 40a-Mn | 7.0 | 1.6 | 3.5 | 6.4 |

The above table shows that the presence of complexes of the present invention considerably improves the bleach performance of peracetic acid on different bleachable stains.

It is also possible to use a combination of TEAD with H₂O₂ instead of peracetic acid to obtain good results.

### Application Example 4

### (Stain bleaching in laundry with oxygen from air as oxidant)

1 g of a circular stain (WFK 10SG tomato-beef sauce, WFK) was added to a vial containing 3 ml of 10 mM carbonate buffer pH 10. The buffer contained 0.6% of a linear alkylbenzenesulfonate. The catalyst concentration (1:1 in-situ complex of the ligand with manganese(II) chloride tetrahydrate in methanolic solution) was 10 and 20 μmol/l, respectively. The vial was shaken with a shaker for 30 minutes at ambient temperature. After the treatment the fabric was rinsed and ironed. The brightness values Y according to the CIE standard procedure of the stained test fabrics was measured with a Gretag SPM 100 instrument prior to and after the treatment (0 h). The stain was then stored in the dark for 24 h and the brightness value was measured once again. The bleaching effect is given as ΔΔY, i.e. the difference in brightness of fabrics treated in the presence vs. the absence of catalyst, respectively.

| | ΔΔY | ΔΔY | ΔΔY | |
|---|---|---|---|---|
| Complex with ligand | (10 μM, | (10 μM, | (20 μM, | ΔΔY (20 μM, |
| example | 0 h) | 24 h) | 0 h) | 24 h) |
| Ligand 28-Mn | 1.1 | 8.8 | 1.2 | 18.5 |

It can be seen that a complex of the present invention is able to effectively bleach stains on fabrics even in the absence of added peroxide.

### Application Example 5

### (Dishwashing)

Tea-stained cups were prepared according to the IKW method (IKW-Arbeitskreis Maschinenspülmittel, “Methoden zur Bestimmung der Reinigungsleistung von maschinellen Geschirrspülmitteln (Part A and B)”, SÖFW, 11+14, 1998). Tea-stained cups were filled with a carbonate buffer solution (pH 9.6) containing 44 mM hydrogen peroxide and 20 μM catalyst (1:1 in-situ complex of the ligand with manganese(II) chloride tetrahydrate in methanolic solution). After 15 minutes the solution was removed, and the cups were rinsed with water. The removal of the tea deposit was evaluated visually on a scale from 0 (i.e. unchanged, very strong deposit) to 10 (i.e. no deposit). A rating of 4.5 was observed in reference experiments without catalyst.

| Complex with ligand example | Rating |
|---|---|
| Ligand 23-Mn | 6.0 |
| Ligand 27-Mn | 6.7 |
| Ligand 28-Mn | 6.6 |
| Ligand 29-Mn | 6.1 |
| Ligand 32-Mn | 6.0 |
| Ligand 33-Mn | 7.1 |
| Ligand 30-Mn | 7.8 |
| Ligand 36-Mn | 6.9 |
| Ligand 39-Mn | 6.3 |
| Ligand 40-Mn | 6.1 |
| Ligand 40a-Mn | 5.9 |
| Ligand 40b-Mn | 5.8 |
| Ligand 40c-Mn | 6.8 |

The table shows that the ratings from experiments with catalysts of the present invention are significantly better than the reference value.

### Application Example 6

### (Delignification of pulp)

5 g (dry weight) of softwood cellulose having a Kappa number of 30 were given into a plastic bag together with 71 ml of carbonate buffer, pH 10 (0.4% sodium hydrogen carbonate and 0.5% sodium carbonate) and 13.3 ml of 30% hydrogen peroxide solution. A catalyst solution (from Ligand 28-Mn) was prepared in situ by dissolving equimolar amounts of ligand and manganese(II) chloride tetrahydrate in methanol. 52.8 ppm of the catalyst was added to the pulp. The pulp so obtained was kneaded intensively and then maintained at 40° C. in a water bath under thermostatic control for 90 min. Filtration was then carried out and the pulp was washed three times with hot water (60° C.). The Kappa number of the pulp after the treatment was determined according to TAPPI T236 om-99 to be κ=20.6. The Kappa number of the pulp in a control experiment without catalyst was κ=23.9. The use of the catalyst of the present invention thus results in a significant delignification of the pulp compared to a control experiment without catalyst.

1. A method of catalyzing an oxidation reaction which comprises oxidizing a substrate in the presence of a catalytically effective amount of at least one metal complex of formula (1)
[LₙMeₘXₚ]^{z}Y_{q} (1),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2) 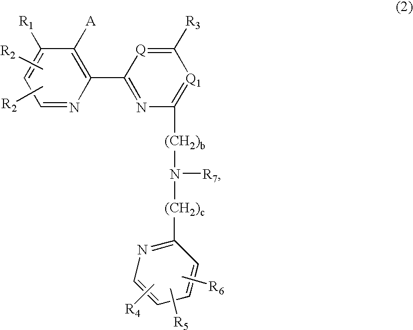
wherein
R₁, R₂, R₃, R₄, R₅, R₆ and R₇ are each independently of the others hydrogen; unsubstituted or substituted C₁-C₁₈alkyl or unsubstituted or substituted aryl; cyano; halogen; nitro; —COOR₉ or —SO₃R₉ wherein
R₉ is in each case hydrogen, a cation or unsubstituted or substituted C₁-C₁₈alkyl or unsubstituted or substituted aryl;
—SR₁₀, —SO₂R₁₀ or —OR₁₀ wherein
R₁₀ is in each case hydrogen or unsubstituted or substituted C₁-C₁₈alkyl or unsubstituted or substituted aryl;
—NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃;
—N(R₁₀)—(C₁-C₆alkylene)-NR₁₁R₁₂; —N[(C₁-C₆alkylene)-NR₁₁R₁₂]₂;
—N(R₁₀)—(C₁-C₆alkylene)-N^{⊕}R₁₁R₁₂R₁₃; —N[(C₁-C₆alkylene)-N^{⊕}R₁₁R₁₂R₁₃]₂; —N(R₁₀)—N—R₁₁R₁₂ or
—N(R₁₀)—N^{⊕}R₁₁R₁₂R₁₃, wherein
R₁₀ is as defined above and
R₁₁, R₁₂ and R₁₃ are each independently of the other(s) hydrogen or unsubstituted or substituted
C₁-C₁₈alkyl or unsubstituted or substituted aryl, or
R₁₁ and R₁₂, together with the nitrogen atom linking them, form an unsubstituted or substituted
5-, 6- or 7-membered ring which may contain further hetero atoms,
Q is N or CR₈, wherein R₈ has the meanings as defined for R₁-R₇ or
R₈ forms together with A a 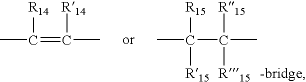
wherein R₁₄, R′₁₄, R₁₅, R′₁₅, R″₁₅ and R′″₁₅ independently from each other are H, C₁-C₄-alkyl or C₁-C₄-alkoxy,
Q₁ is N or CR′₈, wherein R′₈ has the meanings as defined for R₁-R₇,
A has one of the meanings as defined for R₁-R₇, or
A forms together with R₈ a 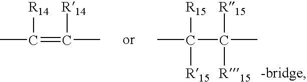
wherein R₁₄, R′₁₄, R₁₅, R′₁₅, R″₁₅ and R′″₁₅ have the same meanings as defined above b and c are each independently from each other 1, 2 or 3.

2. A method according to claim 1, wherein at least one metal complex of formula (1′),
[L′ₙMe_{m′}X′_{p′}]^{z}Y′_{q} (1′)
wherein
Me′ is manganese, titanium, iron, cobalt, nickel or copper,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1 or 2,
p′ is an integer having a value of from 0 to 4,
z′ is an integer having a value of from 8− to 8+,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer from 0 to 8, preferably from 0 to 4,
L′ is a ligand of formula (2a), (2b) or (2c) 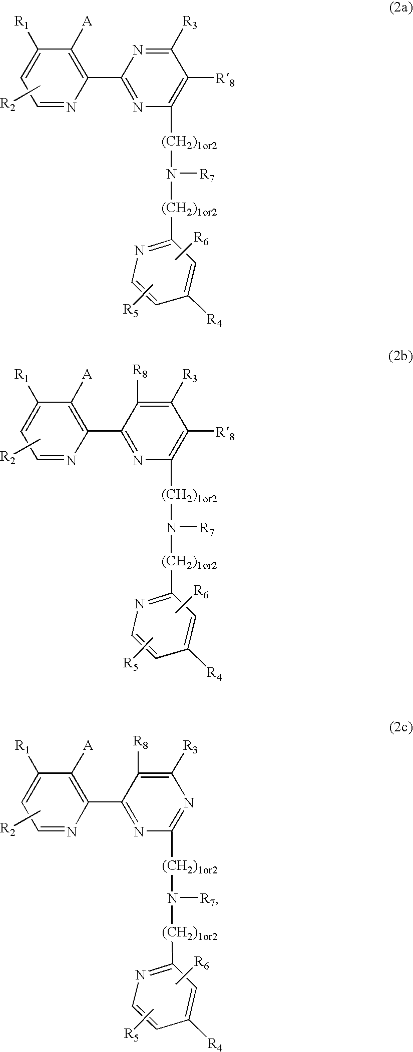
wherein all substituents have the same meanings as defined in claim 1,
is used.

3. A method according to claim 1, wherein at least one metal complex of formula (1′),
[L′ₙMe′_{m′}X′_{p′}]^{z}Y′_{q} (1′)
wherein
Me′ is manganese or iron,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of from 0 to 4+,
Y′ is R₁₇COO⁻; ClO₄⁻; BF₄⁻; PF₆⁻; R₁₇SO₃—; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer from 0 to 4,
L′ is a ligand of formula (2′a), (2′b) or (2′d) 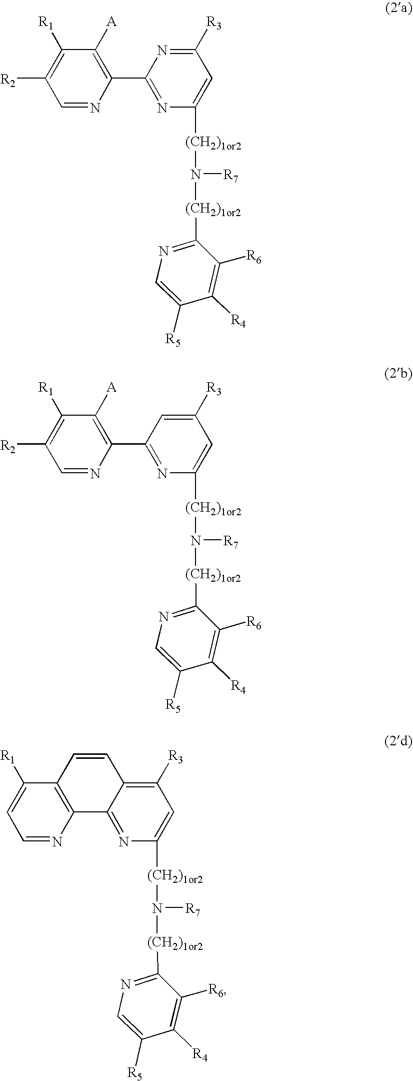
wherein
R₁ and R₄, are independently from each other H; —CH₃; —Cl; —OH; —OCH₃; —NH₂; —N(CH₃)₂;
—N(CH₂CH₃)₂; —N(CH₃)(CH₂CH₃); 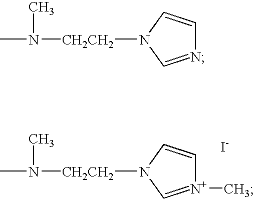
—N(CH₂CH₂OH)₂; —N(CH₂CH₃)(CH₂CH₂OH); —N(CH₃)CH₂CH₂OH; —N(CH₃)CH₂CH₂NH₂; 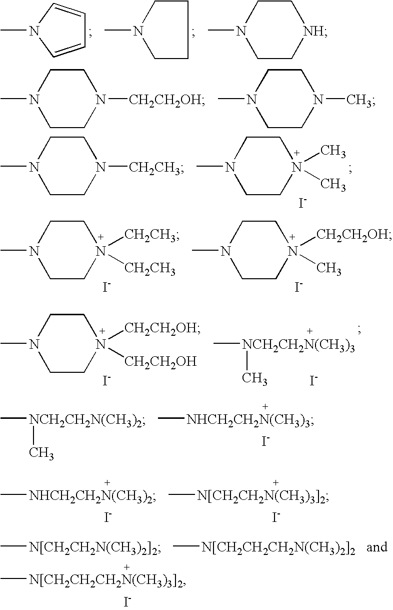
A and R₂, are independently from each other H or —CH₃,
R₃ is —OH; —OCH₃; —NH₂; —N(CH₃)₂; —N(CH₂CH₃)₂; —N(CH₃)(CH₂CH₃); 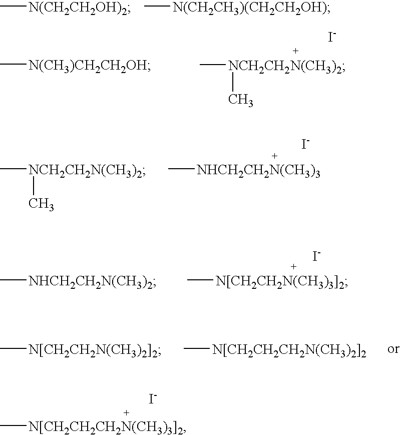
R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and
R₇ is H; —CH₃; —CH₂COOH; —CH₂CH₂COOH; —CH₂CN or —CH₂CH₂CN,
is used.

4. A method according to claim 1 wherein the metal complex compounds of formula (1) are used as catalysts together with peroxide or a peroxide-forming substance, O₂ and/or air for the bleaching of stains or of soiling on textile material in the context of a washing process or by the direct application of a stain remover; for the cleaning of hard surfaces, wall tiles or floor tiles; for the use in automatic dishwashing compositions; for the bleaching of stains or of soiling on textile material by atmospheric oxygen, whereby the bleaching is catalysed during and/or after the treatment of the textile in the washing liquor; for the prevention of redeposition of migrating dyes during the washing of textile material; for the use in washing and cleaning solutions having an antibacterial action; as pretreatment agents for bleaching textiles; as catalysts in selective oxidation reactions in the context of organic synthesis; for the waste water treatment; for bleaching in the context of paper-making; for sterilization; and for contact lens disinfection.

5. A detergent, cleaning, disinfecting or bleaching composition comprising
I) from 0 to 50 wt-%, based on the total weight of the composition, A) of at least one anionic surfactant and/or B) of a non-ionic surfactant,
II) from 0 to 70 wt-%, based on the total weight of the composition, C) of at least one builder substance,
III) 1-99 wt-%, based on the total weight of the composition, D) of at least one peroxide and/or one peroxide-forming substance, O₂ and/or air,
IV) E) at least one metal complex compound of formula (1) or (1′) as defined in claim 1 in an amount that, in the liquor, gives a concentration of from 0.5 to 100 mg/litre of liquor, when from 0.5 to 50 g/litre of the detergent, cleaning, disinfecting or bleaching agent are added to the liquor,
V) 0-20 wt-%, based on the total weight of the composition, of at least one further additive, and
VI) water ad 100 wt-%, based on the total weight of the composition.

6. A composition according to claim 5 comprising
I) from 0 to 30 wt-%, based on the total weight of the composition, A) of at least one anionic surfactant and/or B) of a non-ionic surfactant,
II) from 0 to 50 wt-%, based on the total weight of the composition, C) of at least one builder substance,
III) from 1-99 wt-%, based on the total weight of the composition, D) of at least one peroxide and/or one peroxide-forming substance, O₂ and/or air,
IV) E) at least one metal complex compound of formula (1) or (1′) as defined in claim 1 in an amount that, in the liquor, gives a concentration of from 1 to 50 mg/litre of liquor, when from 0.5 to 50 g/litre of the detergent, cleaning, disinfecting or bleaching agent are added to the liquor,
V) from 0-20 wt-%, based on the total weight of the composition, of at least one further additive, and
VI) water ad 100 wt-%, based on the total weight of the composition.

7. A composition according to claim 6 comprising
I) from 1-50 wt-%, based on the total weight of the composition, A) of at least one anionic surfactant and/or B) of at least one non-ionic surfactant,
II) from 1-70 wt-%, based on the total weight of the composition, C) of at least one builder substance,
III) from 1-99 wt-%, based on the total weight of the composition, of at least one peroxide and/or of at least one peroxide-forming substance, O₂ and/or air,
IV) from 0.005-2 wt-%, based on the total weight of the composition, E) of at least one metal complex compound of formula (1) or (1′) as defined in claim 1
V) from 0-20 wt-%, based on the total weight of the composition, of at least one further additive, and
VI) water ad 100 wt-%, based on the total weight of the composition.

8. A composition according to claim 5, which is used on a textile material or hardsurface material.

9. A composition according to claim 5, which is a dishwashing detergent formulation.

10. A composition according to claim 9, which is an automatic dishwashing detergent formulation.

11. A granule comprising
a) from 1-99 wt-%, based on the total weight of the granule, of at least one metal complex compound of formula (1) as defined in claim 1 and of at least one peroxide,
b) from 1-99 wt-%, based on the total weight of the granule, of at least one binder,
c) from 0-20 wt-%, based on the total weight of the granule, of at least one encapsulating material,
d) from 0-20 wt-%, based on the total weight of the granule, of at least one further additive and
e) from 0-20 wt-% based on the total weight of the granule, of water.

12. A granule comprising
a) from 1-99 wt-%, based on the total weight of the granule, of at least one metal complex compound of formula (1) as defined in claim 1 and of at least one peroxide-forming substance,
b) from 1-99 wt-%, based on the total weight of the granule, of at least one binder,
c) from 0-20 wt-%, based on the total weight of the granule, of at least one encapsulating material,
d) from 0-20 wt-%, based on the total weight of the granule, of at least one further additive and
e) from 0-20 wt-% based on the total weight of the granule, of water.

13. Metal complexes of formula (1)
[LₙMeₘXₚ]^{z}Y_{q} (1),
wherein
Me is manganese, titanium, iron, cobalt, nickel or copper,
X is a coordinating or bridging radical,
n and m are each independently of the other an integer having a value of from 1 to 8,
p is an integer having a value of from 0 to 32,
z is the charge of the metal complex,
Y is a counter-ion,
q=z/(charge of Y), and
L is a ligand of formula (2) 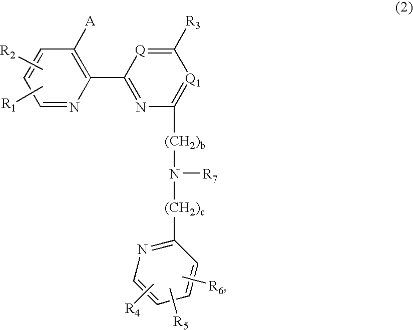
wherein
R₁, R₂, R₃, R₄, R₅, R₆ and R₇ are each independently of the others hydrogen; unsubstituted or substituted C₁-C₁₈alkyl or unsubstituted or substituted aryl; cyano; halogen; nitro; —COOR₉ or —SO₃R₉ wherein
R₉ is in each case hydrogen, a cation or unsubstituted or substituted C₁-C₁₈alkyl or unsubstituted or substituted aryl;
—SR₁₀, —SO₂R₁₀ or —OR₁₀ wherein
R₁₀ is in each case hydrogen or unsubstituted or substituted C₁-C₁₈alkyl or unsubstituted or substituted aryl;
—NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃;
—N(R₁₀)—(C₁-C₆alkylene)-NR₁₁R₁₂; —N[(C₁-C₆alkylene)-NR₁₁R₁₂]₂;
—N(R₁₀)—(C₁-C₆alkylene)-N^{⊕}R₁₁R₁₂R₁₃; —N[(C₁-C₆alkylene)-N^{⊕}R₁₁R₁₂R₁₃]₂; —N(R₁₀)—N—R₁₁R₁₂ or
—N(R₁₀)—N^{⊕}R₁₁R₁₂R₁₃, wherein
R₁₀ is as defined above and
R₁₁, R₁₂ and R₁₃ are each independently of the other(s) hydrogen or unsubstituted or substituted
C₁-C₁₈alkyl or unsubstituted or substituted aryl, or
R₁₁ and R₁₂, together with the nitrogen atom linking them, form an unsubstituted or substituted
5-, 6- or 7-membered ring which may contain further hetero atoms,
Q is N or CR₈, wherein R₈ has the meanings as defined for R₁-R₇ or
R₈ forms together with A a 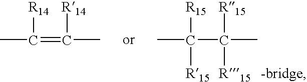
wherein R₁₄, R′₁₄, R₁₅, R′₁₅, R″₁₅ and R′″₁₅ independently from each other are H, C₁-C₄-alkyl or C₁-C₄-alkoxy,
Q₁ is N or CR′₈, wherein R′₈ has the meanings as defined for R₁-R₇,
A has one of the meanings as defined for R₁-R₇, or
A forms together with R₈ a 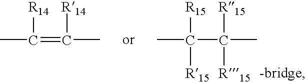
wherein R₁₄, R′₁₄, R₁₅, R′₁₅, R″₁₅ and R′″₁₅ have the same meanings as defined above b and c are each independently from each other 1, 2 or 3.

14. Metal complex of formula (1′) according to claim 13,
[L′ₙMe′_{m′}X′_{p′}]^{z}Y′_{q} (1′)
wherein
Me′ is manganese or iron,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of from 0 to 4+,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer from 0 to 4,
L′ is a ligand of formula (2′a), (2′b) or (2′d) 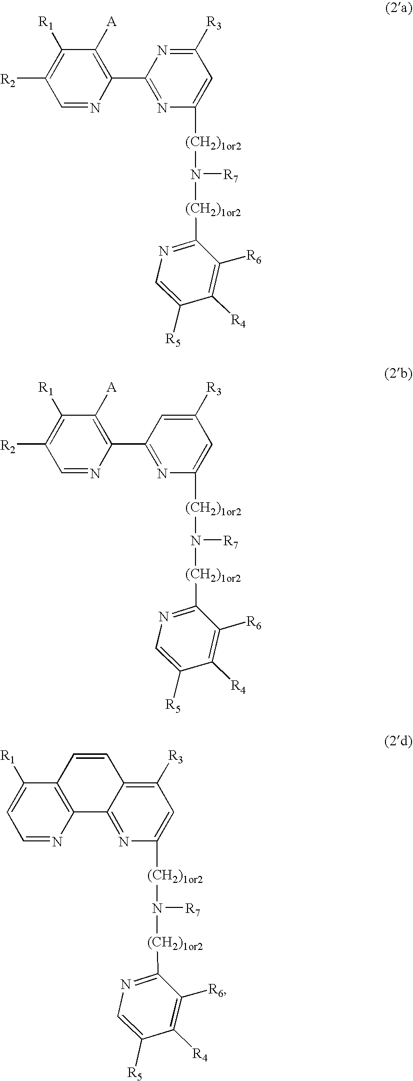
wherein
R₁ and R₄, are independently from each other H; —CH₃; —Cl; —OH; —OCH₃; —NH₂; —N(CH₃)₂;
—N(CH₂CH₃)₂; —N(CH₃)(CH₂CH₃); 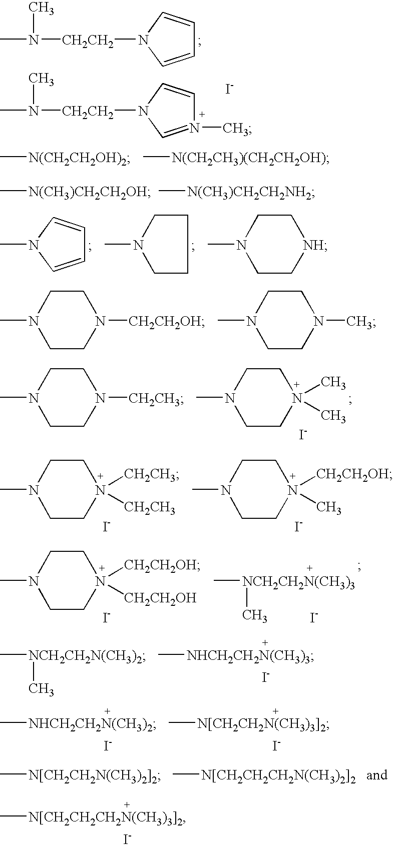
A and R₂, are independently from each other H or —CH₃,
R₃ is —OH; —OCH₃; —NH₂; —N(CH₃)₂; —N(CH₂CH₃)₂; —N(CH₃)(CH₂CH₃); 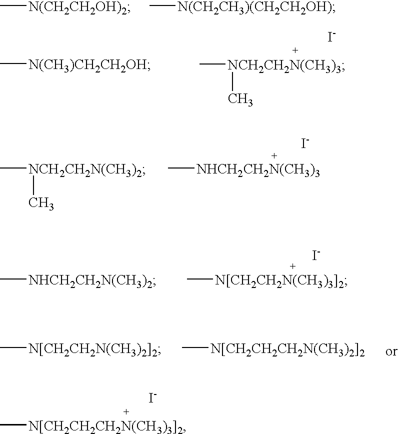
R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and
R₇ is H; —CH₃; —CH₂COOH; —CH₂CH₂COOH; —CH₂CN or —CH₂CH₂CN.

15. Ligands of formula (2) 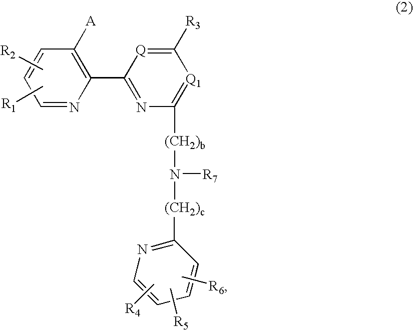
wherein
R₁, R₂, R₃, R₄, R₅, R₆ and R₇ are each independently of the others hydrogen; unsubstituted or substituted C₁-C₁₈alkyl or unsubstituted or substituted aryl; cyano; halogen; nitro; —COOR₉ or —SO₃R₉ wherein
R₉ is in each case hydrogen, a cation or unsubstituted or substituted C₁-C₁₈alkyl or unsubstituted or substituted aryl;
—SR₁₀, —SO₂R₁₀ or —OR₁₀ wherein
R₁₀ is in each case hydrogen or unsubstituted or substituted C₁-C₁₈alkyl or unsubstituted or substituted aryl;
—NR₁₁R₁₂; —(C₁-C₆alkylene)-NR₁₁R₁₂; —N^{⊕}R₁₁R₁₂R₁₃; —(C₁-C₆alkylene)-N^{⊕}R₁₁R₁₂R₁₃;
—N(R₁₀)—(C₁-C₆alkylene)-NR₁₁R₁₂; —N[(C₁-C₆alkylene)-NR₁₁R₁₂]₂;
—N(R₁₀)—(C₁-C₆alkylene)-N^{⊕}R₁₁R₁₂R₁₃; —N[(C₁-C₆alkylene)-N^{⊕}R₁₁R₁₂R₁₃]₂; —N(R₁₀)—N—R₁₁R₁₂ or
—N(R₁₀)—N^{⊕}R₁₁R₁₂R₁₃, wherein
R₁₀ is as defined above and
R₁₁, R₁₂ and R₁₃ are each independently of the other(s) hydrogen or unsubstituted or substituted
C₁-C₁₈alkyl or unsubstituted or substituted aryl, or
R₁₁ and R₁₂, together with the nitrogen atom linking them, form an unsubstituted or substituted
5-, 6- or 7-membered ring which may contain further hetero atoms,
Q is N or CR₈, wherein R has the meanings as defined for R₁-R₇ or
R₈ forms together with A a 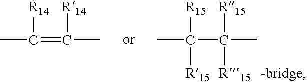
wherein R₁₄, R′₁₄, R₁₅, R′₁₅, R″₁₅ and R′″₁₅ independently from each other are H, C₁-C₄-alkyl or C₁-C₄-alkoxy,
Q₁ is N or CR′₈, wherein R′₈ has the meanings as defined for R₁-R₇,
A has one of the meanings as defined for R₁-R₇, or
A forms together with R₈ a 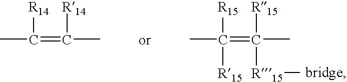
wherein R₁₄, R′₁₄, R₁₅, R′₁₅, R″₁₅ and R′″₁₅ have the same meanings as defined above b and c are each independently from each other 1, 2 or 3.

16. Ligands of formula (2′a), (2′b) or (2′d) according to claim 15 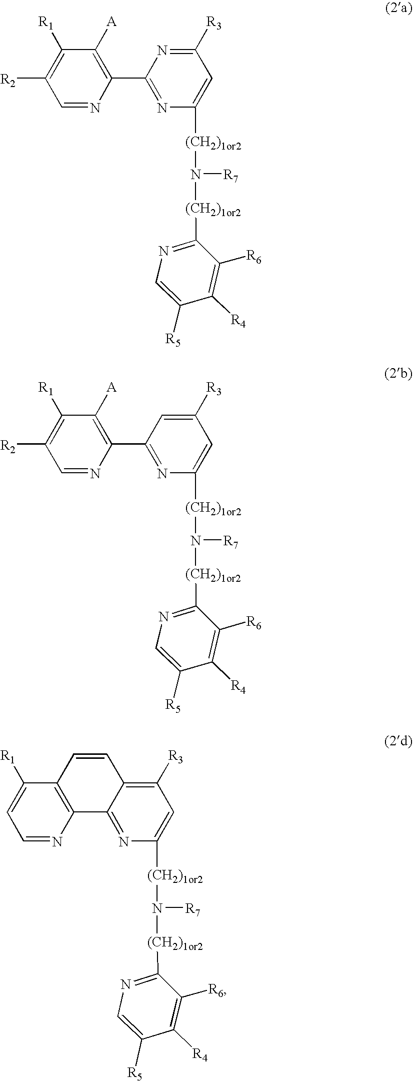
wherein
R₁ and R₄, are independently from each other H; —CH₃; —Cl; —OH; —OCH₃; —NH₂; —N(CH₃)₂;
—N(CH₂CH₃)₂; —N(CH₃)(CH₂CH₃); 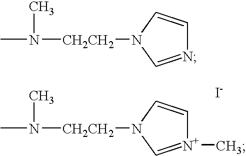
—N(CH₂CH₂OH)₂; —N(CH₂CH₃)(CH₂CH₂OH); —N(CH₃)CH₂CH₂OH; —N(CH₃)CH₂CH₂NH₂; 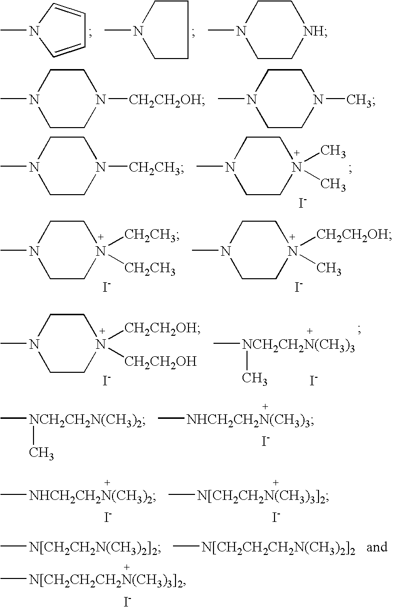
A and R₂, are independently from each other H or —CH₃,
R₃ is —OH; —OCH₃; —NH₂; —N(CH₃)₂; —N(CH₂CH₃)₂; —N(CH₃)(CH₂CH₃); 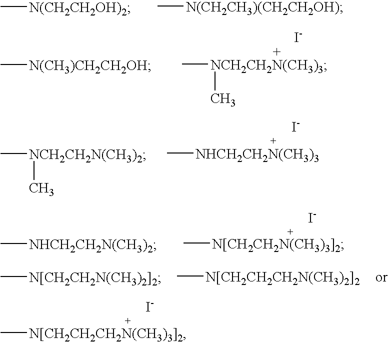
R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and
R₇ is H; —CH₃; —CH₂COOH; —CH₂CH₂COOH; —CH₂CN or —CH₂CH₂CN.

17. A process for production of compounds of formula (2) according to the following reaction scheme: 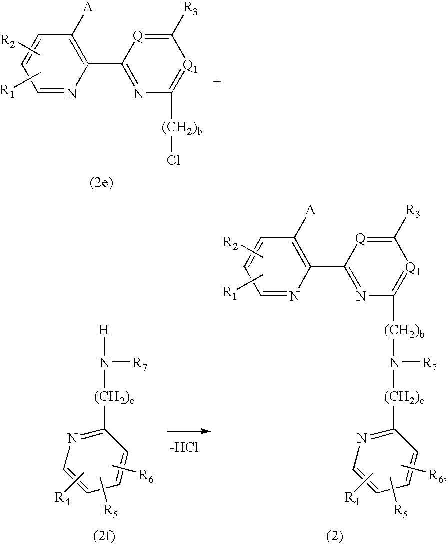
wherein all the substituents have the meanings as defined in claim 1.

18. A method according to claim 3, wherein at least one metal complex of formula (1′),
[L′ₙMe′_{m′}X′_{p′}]^{z}Y′_{q} (1′)
wherein
Me′ is manganese or iron,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of 0,
Y′ is R₁₇COO⁻; ClO₄⁻; BF₄⁻; PF₆⁻; R₁₇SO₃—; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer having a value of 0,
L′ is a ligand of formula (2′a), (2′b) or (2′d) 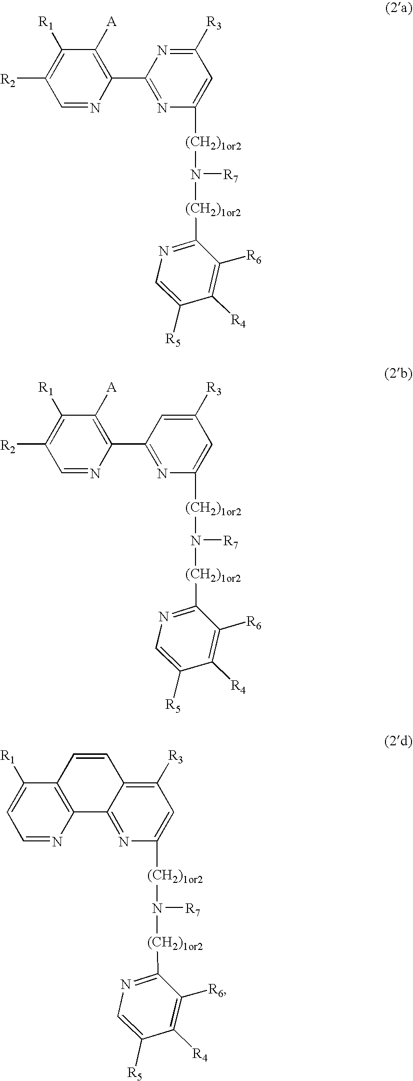
wherein
R₁ and R₄, are independently from each other H; —CH₃; —Cl; —OH; —OCH₃; —NH₂; —N(CH₃)₂;
—N(CH₂CH₃)₂; —N(CH₃)(CH₂CH₃); 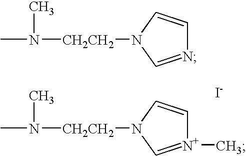
—N(CH₂CH₂OH)₂; —N(CH₂CH₃)(CH₂CH₂OH); —N(CH₃)CH₂CH₂OH; —N(CH₃)CH₂CH₂NH₂; 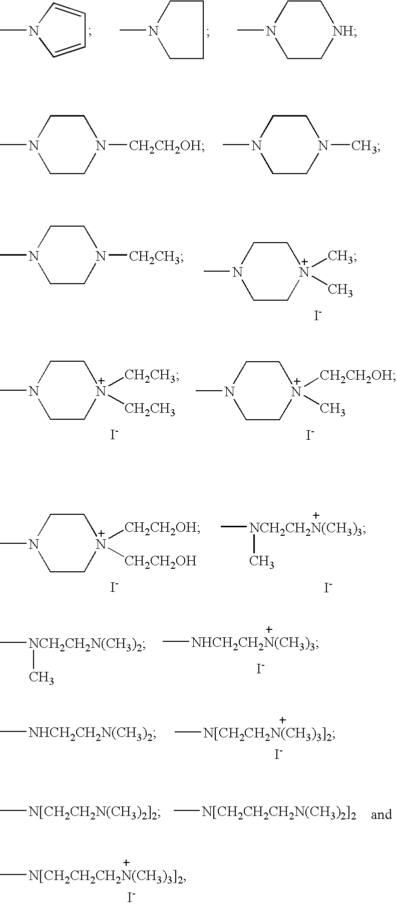
A and R₂, are independently from each other H or —CH₃,
R₃ is —OH; —OCH₃; —NH₂; —N(CH₃)₂; —N(CH₂CH₃)₂; —N(CH₃)(CH₂CH₃); 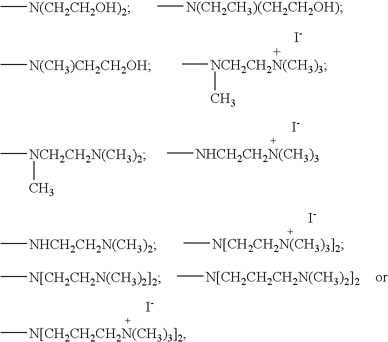
R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and
R₇ is H; —CH₃; —CH₂COOH; —CH₂CH₂COOH; —CH₂CN or —CH₂CH₂CN,
is used.

19. Metal complex of formula (1′) according to claim 14,
[L′ₙMe′_{m′}X′_{p′}]^{z}Y′_{q} (1′)
wherein
Me′ is manganese or iron,
X′ is CH₃CN; H₂O; F⁻; Cl⁻; Br⁻; HOO⁻; O₂²⁻; O²⁻; R₁₆COO⁻; or R₁₆O⁻, wherein R₁₆ is hydrogen, C₁-C₄alkyl, sulphophenyl or phenyl,
n′ is an integer having a value of 1 or 2,
m′ is an integer having a value of 1,
p′ is an integer having a value of 2,
z′ is an integer having a value of 0,
Y′ is R₁₇COO⁻; ClO₄⁻; BF₄⁻; PF₆⁻; R₁₇SO₃⁻; R₁₇SO₄⁻; SO₄²⁻; NO₃⁻; F⁻; Cl⁻; Br⁻, I⁻, citrate, oxalate or tartrate, wherein R₁₇ is hydrogen; C₁-C₄alkyl; phenyl, or sulfophenyl,
q′ is an integer having a value of 0,
L′ is a ligand of formula (2′a), (2′b) or (2′d) 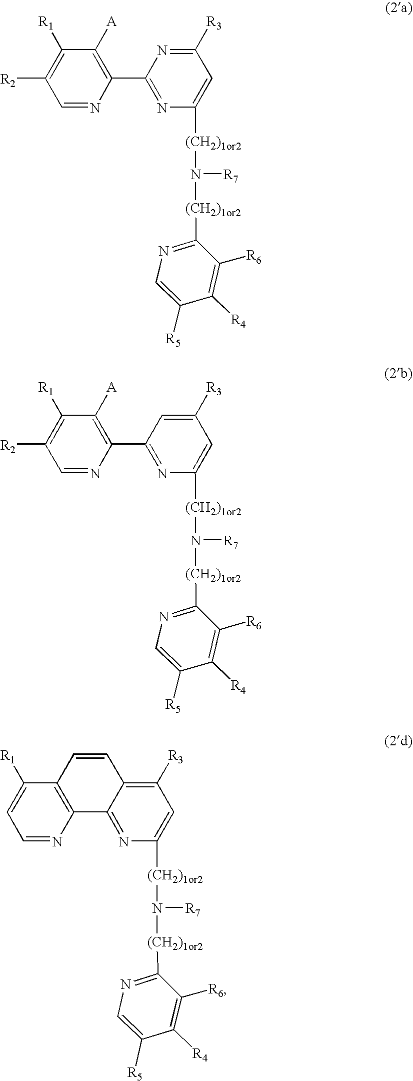
wherein
R₁ and R₄, are independently from each other H; —CH₃; —Cl; —OH; —OCH₃; —NH₂; —N(CH₃)₂;
—N(CH₂CH₃)₂; —N(CH₃)(CH₂CH₃); 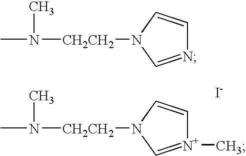
—N(CH₂CH₂OH)₂; —N(CH₂CH₃)(CH₂CH₂OH); —N(CH₃)CH₂CH₂OH; —N(CH₃)CH₂CH₂NH₂; 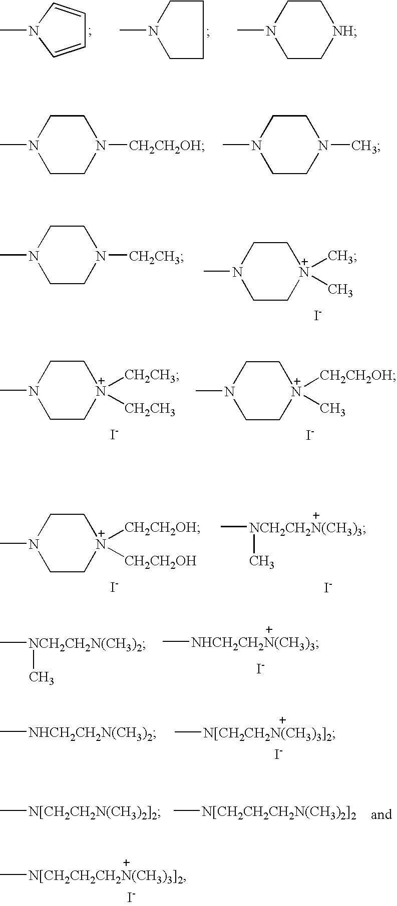
A and R₂, are independently from each other H or —CH₃,
R₃ is —OH; —OCH₃; —NH₂; —N(CH₃)₂; —N(CH₂CH₃)₂; —N(CH₃)(CH₂CH₃); 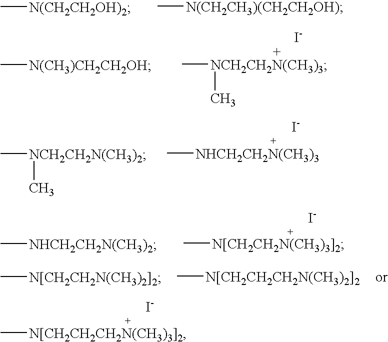
R₅ and R₆ are independently from each other hydrogen; —CH₃; —Cl; —NH₂; —N(CH₃)₂;
—N(CH₂CH₃)₂; —N(CH₃)(CH₂CH₃); —N(CH₂CH₂OH)₂; —N(CH₂CH₃)(CH₂CH₂OH) or
—N(CH₃)CH₂CH₂OH, and
R₇ is H; —CH₃; —CH₂COOH; —CH₂CH₂COOH; —CH₂CN or —CH₂CH₂CN.
